(19) United States
(12) Patent Application Publication
(10) Pub. No.: US 2016/0003799 A1
(43) Pub. Date: Jan. 7, 2016

(54) Means and Methods for Assessing the Quality of a Biological Sample

(71) Applicant: METANOMICS HEALTH GMBH, Berlin (DE)

(72) Inventors: Beate Kamlage, Berlin (DE); Oliver Schmitz, Dallgow-Dobertiz (DE); Jürgen Kastler, Berlin (DE); Gareth Catchpole, Potsdam (DE); Martin Dostler, Henningsdorf (DE); Volker Liebenberg, Berlin (DE)

(73) Assignee: Metanomics Health GMBH, Berlin (DE)

(21) Appl. No.: 14/767,059

(22) PCT Filed: Feb. 14, 2014
(86) PCT No.: PCT/IB2014/059002

§ 371 Date: Aug. 11, 2015

Related U.S. Application Data
(60) Provisional application No. 61/764,625, filed on Feb. 14, 2013.
(60) Provisional application No. 61/764,640, filed on Feb. 14, 2013.

(30) Foreign Application Priority Data
Feb. 14, 2013 (EP) 13155310.9
Feb. 14, 2013 (EP) 13155318.2

(51) Int. Cl. G01N 33/49

(52) U.S. Cl. CPC G01N 33/492; G01N 2570/00; G01N 2560/00; G06F 19/18

(57) ABSTRACT

The present invention relates to the field of diagnostic methods. Specifically, the present invention relates to a method for assessing the quality of a biological sample comprising the steps of determining in a sample the amount of at least one biomarker from Tables 1, 1′, 2, 2′, 3, 3′, 4, 5, 5′, 6, 7, and/or 8 and comparing the said amount of the at least one biomarker with a reference, whereby the quality of the sample is assessed. The invention also relates to tools for carrying out the aforementioned method, such as devices and kits.

The present invention relates to the field of diagnostic methods. Specifically, the present invention relates to a method for assessing the quality of a biological sample comprising the steps of determining in a sample the amount of at least one biomarker from Tables 1, 1′, 2, 2′, 3, 3′, 4, 5, 5′, 6, 7, and/or 8 and comparing the said amount of the at least one biomarker with a reference, whereby the quality of the sample is assessed. The invention also relates to tools for carrying out the aforementioned method, such as devices and kits.

The value of biological material stored in biobanks for any biomedical research related to metabolite profiling, e.g., the potential of biomarker identification and validation, is diminished by pre-analytical confounding factors that interfere with the sample metabolome and may lead to unbalanced study design, increased variability, erratic effects and irreproducible results. It is decisive to assess the quality of biological material in order to assure quality and suitability for metabolite profiling or other analytical or diagnostic methods. Specifically, confounding factors of relevance are increased time and temperature of blood, plasma or serum sample processing and storage, effects of centrifugation protocol, hemolysis, contamination with blood cells, e.g. by dispersing the buffy layer or the blood clot after centrifugation, freezing protocol, microclotting of blood samples destined for plasma preparation due to e.g. delayed or insufficient mixture of blood with the anticoagulant, and other pre-analytical steps.

There are various standards for quality assurance and quality control for biobanking, e.g., ISO 9001, ISO guide 34, ISO 17025 and others (see, e.g., Carter 2011, Biopreservation and Biobanking 9(2): 157-163; Elliott 2008, Int J Epidemiology 37: 234-244). In order to assess the quality of biological material, at present, biochemical standard parameters, such as nucleic acid content and integrity, presence of coagulation activity, or cellular composition, cell integrity and number of cells in the sample are determined. The evaluation of such standard parameters, however, will not be suitable for a more defined quality assessment for metabolome analysis.

There are reports of protein biomarkers assuring quality of samples for proteome analysis (see, e.g., WO2012/170669). Moreover, it was reported that incubation has an impact on the metabolomic composition of plasma and serum samples (Liu et al. 2010, Anal Biochem 406: 105-115; Fliniaux et al. 2011, Journal of Biomolecular NMR 51(4): 457-465; Boyanton 2002, Clinical Chemistry 48(12): 2242-2247; Bernini et al. 2011, Journal of Biomolecular NMR 49: 231-243).

However, standards for assessing the metabolome quality of biological material are not yet available but nevertheless highly desired.

The technical problem underlying the present invention can be seen as the provision of means and methods for complying with the aforementioned needs. The technical problem is solved by the embodiments characterized in the claims and herein below.

Thus, the present invention relates to a method for assessing the quality of a biological sample comprising the steps of:
(a) determining in said sample the amount of at least one biomarker from Tables 1, 1′, 2, 2′, 3, 3′, 4, 5, 5′, 6, 7, and/or 8; and
(b) comparing the said amount of the at least one biomarker with a reference, whereby the quality of the sample is assessed.

Preferably, the present invention relates to a method for assessing the quality of a biological sample comprising the steps of:
(a) determining in said sample the amount of at least one biomarker from Tables 1, 2, 3, 4, 5, 6, 7 and/or 8; and
(b) comparing the said amount of the at least one biomarker with a reference, whereby the quality of the sample is assessed.

The method as referred to in accordance with the present invention includes a method which essentially consists of the aforementioned steps or a method which includes further steps. However, it is to be understood that the method, in a preferred embodiment, is a method carried out ex vivo, i.e. not practised on the human or animal body. The method, preferably, can be assisted by automation.

In preferred embodiments, the method of the present invention comprises one or more of the following steps: i) contacting said biological sample with an agent specifically interacting with at least one biomarker of the present invention, and determining the amount of a complex formed between said biomarker and said agent specifically interacting with said biomarker; ii) contacting said biological sample with an enzyme specifically reacting with said at least one biomarker of the present invention, and determining the amount of product formed from said biomarker by said enzyme; iii) contacting said biological sample with an agent modifying the chemical structure of at least one biomarker, preferably, to form a non-naturally occurring derivative of said biomarker, and detecting said derivative; iv) discarding said sample in case insufficient quality is assessed, and v) excluding said sample from further analysis in case insufficient quality is assessed.

The term “assessing” as used herein refers to distinguishing between insufficient and sufficient quality of a sample for metabolic analysis. Insufficient quality of a sample as used herein refers to a composition of a sample which does not allow for a proper analysis of the metabolomic composition, while samples of sufficient quality allow for proper analysis of the metabolomic composition. A sample being of insufficient quality may cause an improper analysis because the metabolic composition is altered with respect to the amounts of metabolites as well as the chemical nature of metabolites. Insufficient quality may be caused, preferably, by degradation of metabolites and/or chemical alterations of the said metabolites. More preferably, the quality of the sample is insufficient because of adverse effects of pre-analytical confounding factors and, preferably, prolonged processing, hemolysis, microclotting, cellular contamination, improper storage conditions and/or improper freezing, preferably slow freezing.

As will be understood by those skilled in the art, such an assessment, although preferred to be, may usually not be correct for 100% of the investigated samples. The term, however, requires that a statistically significant portion of samples can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, or 0.05.

The term “biomarker” as used herein refers to a molecular species which serves as an indicator for a quality impairment or status as referred to in this specification. Said molecular species can be a metabolite itself which is found in a sample of a subject. Moreover, the biomarker may also be a molecular species which is derived from said metabolite. In such a case, the actual metabolite will be chemically modified in the sample or during the determination process and, as a result of said modification, a chemically different molecular species, i.e. the analyte, will be the determined molecular species. It is to be understood that in such a case, the analyte represents the actual metabolite and has the same potential as an indicator for the respective quality impairment.

Moreover, a biomarker according to the present invention is not necessarily corresponding to one molecular species. Rather, the biomarker may comprise stereoisomers or enantiomeres of a compound. Further, a biomarker can also represent the sum of isomers of a biological class of isomeric molecules. Said isomers shall exhibit identical analytical characteristics in some cases and are, therefore, not distinguishable by various analytical methods including those applied in the accompanying Examples described below. However, the isomers will share at least identical sum formula parameters and, thus, in the case of, e.g., lipids an identical chain length and identical numbers of double bonds in the fatty acid and/or sphingo base moieties.

Polar biomarkers can be, preferably, obtained by techniques referred to in this specification elsewhere and as described in Examples, below. Lipid biomarkers can be obtained in accordance with the present invention, preferably, as described in this specification elsewhere and, in particular, either as lipid fraction by separation of a sample after protein precipitation into an aqueous polar and an organic lipid phase by, e.g., a mixture of ethanol and dichloromethane as described in Examples, below. Those biomarkers may be marked by “lipid fraction” herein. Alternatively or in addition, biomarkers may be enriched from the sample using solid phase extraction (SPE).

In the method according to the present invention, at least one metabolite of the biomarkers shown in Tables 1, 1′, 2, 2′, 3, 3′, 4, 5, 5′, 6, 7, and/or 8 is to be determined. More preferably, at least one metabolite of the biomarkers shown in Tables 1a, 1b, 1c, 1d, 1a′, 1c′, 1d′, 2a, 2b, 2c, 2d, 2a′, 2b′, 2c′, 2d′, 3a, 3c, 3a′, 3c′, 4a, 4b, 4c, 4d, 5a, 5b, 5c, 5d, 5′, 6a, 6b, 6c, 6d, 7a, 7c, 8a, 8b, 8c, and/or 8d is to be determined. Even more preferably, at least one metabolite of the biomarkers shown in Tables 1, 2, 3, 4, 5, 6, 7 and/or 8 is to be determined. Most preferably, at least one metabolite of the biomarkers shown in Tables 1a, 1b, 1c, 1 d, 2a, 2b, 2c, 2d, 3a, 3c, 4a, 4b, 4c, 4d, 5a, 5b, 5c, 5d, 6a, 6b, 6c, 6d, 7a, 7c, 8a, 8b, 8c, and/or 8d is to be determined.

Preferably, in the method according to the present invention, a group of biomarkers will be determined in order to strengthen specificity and/or sensitivity of the assessment. Such a group, preferably, comprises at least 2, at least 3, at least 4, at least 5, at least 10 or up to all of the said biomarkers shown in the said Tables. Preferably, in the method of the present invention, at least one biomarker per Table number is to be determined, i.e. at least one biomarker per Table X or X′, wherein X=1, 2, 3, 4, 5, 6, 7, 8. More preferably, in the method of the present invention, at least one biomarker per Table X is to be determined, i.e. at least one biomarker from any one of Tables 1, 2, 3, 4, 5, 6, 7 and/or 8.

A metabolite as used herein refers to at least one molecule of a specific metabolite up to a plurality of molecules of the said specific metabolite. It is to be understood further that a group of metabolites means a plurality of chemically different molecules wherein for each metabolite at least one molecule up to a plurality of molecules may be present. A metabolite in accordance with the present invention encompasses all classes of organic or inorganic chemical compounds including those being comprised by biological material such as organisms. Preferably, the metabolite in accordance with the present invention is a small molecule compound. More preferably, in case a plurality of metabolites is envisaged, said plurality of metabolites representing a metabolome, i.e. the collection of metabolites being comprised by an organism, an organ, a tissue, a body fluid or a cell at a specific time and under specific conditions.

In addition to the specific biomarkers recited in the specification, other biomarkers and/or indicators may be, preferably, determined as well in the methods of the present invention. Such biomarkers may include peptide or polypeptide biomarkers, e.g., those referred to in WO2012/170669, Liu 2010 loc cit, or Fliniaux 2011, loc cit.

The term “sample” as used herein refers to samples comprising biological material and, in particular, metabolic biomarkers including those referred to herein. Preferably, a sample in accordance with the present invention is a sample from body fluids, preferably, blood, plasma, serum, saliva or urine, or a sample derived, e.g., by biopsy, from cells, tissues or organs. More preferably, the sample is a blood, plasma or serum sample, most preferably, a plasma sample. The aforementioned samples can be derived from a subject as specified elsewhere herein. Techniques for obtaining the aforementioned different types of biological samples are well known in the art. For example, blood samples may be obtained by blood taking while tissue or organ samples are to be obtained, e.g., by biopsy.

The aforementioned samples are, preferably, pre-treated before they are used for the method of the present invention. As described in more detail below, said pre-treatment may include treatments required to release or separate the compounds or to remove excessive material or waste. Furthermore, pre-treatments may aim at sterilizing samples and/or removing contaminants such as undesired cells, bacteria or viruses. Suitable techniques comprise centrifugation, extraction, fractioning, ultrafiltration, protein precipitation followed by filtration and purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Another kind of pre-treatment may be the storage of the samples under suitable storage conditions. Storage conditions as referred to herein include storage temperature, pressure, humidity, time as well as the treatment of the stored samples with preserving agents. Suitable and necessary pre-treatments also depend on the means used for carrying out the method of the invention and are well known to the person skilled in the art. Pre-treated samples as described before are also comprised by the term “sample” as used in accordance with the present invention.

The sample referred to in accordance with the present invention can, preferably, be derived from a subject. A subject as used herein relates to animals and, preferably, to mammals. More preferably, the subject is a rodent and, most preferably, a mouse or rat or a primate and, most preferably, a human. The subject, preferably, is suspected to suffer from a disease or medical condition, or not, or be at risk for developing a disease or medical condition, or not.

The term “determining the amount” as used herein refers to determining at least one characteristic feature of a biomarker to be determined by the method of the present invention in the sample. Characteristic features in accordance with the present invention are features which characterize the physical and/or chemical properties including biochemical properties of a biomarker.

Such properties include, e.g., molecular weight, viscosity, density, electrical charge, spin, optical activity, colour, fluorescence, chemoluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the said at least one biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z information, as well as an intensity value being related to the abundance of the said biomarker (i.e. its amount) in the sample.

As discussed before, each biomarker comprised by a sample may be, preferably, determined in accordance with the present invention quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is enlarged or diminished with respect to a second sample comprising said biomarker in a second amount. In a preferred embodiment said second sample comprising said biomarker shall be a calculated reference as specified elsewhere herein. Quantitatively analysing a biomarker, thus, also includes what is sometimes referred to as semi-quantitative analysis of a biomarker.

Moreover, determining as used in the method of the present invention, preferably, includes using a compound separation step prior to the analysis step referred to before. Preferably, said compound separation step yields a time resolved separation of the metabolites comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the method of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FTIR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

Moreover, the at least one biomarker can also be determined by a specific chemical or biological assay. Said assay shall comprise means which allow to specifically detect the at least one biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, antibodies or other proteins which specifically interact with chemical structures, such as receptors or enzymes. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)₂ fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the biomarker are, preferably, enzymes which are involved in the metabolic conversion of the said biomarker. Said enzymes may either use the biomarker as a substrate or may convert a substrate into the biomarker. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Moreover, the biomarker may also be determined based on its capability to react with other compounds, i.e. by a specific chemical reaction. Further, the biomarker may be determined in a sample due to its capability to elicit a response in a biological read out system. The biological response shall be detected as read out indicating the presence and/or the amount of the biomarker comprised by the sample. The biological response may be, e.g., the induction of gene expression or a phenotypic response of a cell or an organism. In a preferred embodiment the determination of the least one biomarker is a quantitative process, e.g., allowing also the determination of the amount of the at least one biomarker in the sample.

As described above, said determining of the at least one biomarker can, preferably, comprise mass spectrometry (MS). Mass spectrometry as used herein encompasses all techniques which allow for the determination of the molecular weight (i.e. the mass) or a mass variable corresponding to a compound, i.e. a biomarker, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. How to apply these techniques is well known to the person skilled in the art. Moreover, suitable devices are commercially available. More preferably, mass spectrometry as used herein relates to LC-MS and/or GC-MS, i.e. to mass spectrometry being operatively linked to a prior chromatographic separation step. More preferably, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) fragmentation of the ion selected in step a) by applying an acceleration voltage in an additional subsequent quadrupole which is filled with a collision gas and acts as a collision chamber, c) selection of a mass/charge quotient of an ion created by the fragmentation process in step b) in an additional subsequent quadrupole, whereby steps a) to c) of the method are carried out at least once and analysis of the mass/charge quotient of all the ions present in the mixture of substances as a result of the ionisation process, whereby the quadrupole is filled with collision gas but no acceleration voltage is applied during the analysis. Details on said most preferred mass spectrometry to be used in accordance with the present invention can be found in WO2003/073464.

More preferably, said mass spectrometry is liquid chromatography (LC) MS and/or gas chromatography (GC) MS. Liquid chromatography as used herein refers to all techniques which allow for separation of compounds (i.e. metabolites) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. Gas chromatography as applied in accordance with the present invention, in principle, operates comparable to liquid chromatography. However, rather than having the compounds (i.e. metabolites) in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time which is required for passing through the column. Moreover, in the case of gas chromatography it is preferably envisaged that the compounds are derivatised prior to gas chromatography. Suitable techniques for derivatisation are well known in the art. Preferably, derivatisation in accordance with the present invention relates to methoxymation and trimethylsilylation of, preferably, polar compounds and transmethylation, methoxymation and trimethylsilylation of, preferably, non-polar (i.e. lipophilic) compounds.

The term “reference” refers to values of characteristic features of each of the biomarker which can be correlated to an insufficient quality of the sample. Preferably, a reference is a threshold value (e.g., an amount or ratio of amounts) for a biomarker whereby said threshold divides the range of possible values for the characteristic features into a first and a second part. One of these parts is associated with insufficient quality while the other is associated with sufficient quality. The threshold value itself may also be associated with either sufficient or insufficient quality. In case the threshold is associated with insufficient quality, values found in a sample to be investigated which are, therefore, essentially identical to the threshold or which fall into the part associated with insufficient quality indicate insufficient quality of the sample. In case the threshold is associated with sufficient quality, values found in a sample to be investigated which are essentially identical to the threshold or which fall into the part associated with sufficient quality indicate sufficient quality of the sample.

In accordance with the aforementioned method of the present invention, a reference is, preferably, a reference obtained from a sample or plurality of samples (i.e., preferably, more than 1, 2, 3, 4, 5, 10, 50 or 100 samples) known to be of insufficient quality. In such a case, a value for the at least one biomarker found in the test sample being essentially identical is indicative for insufficient quality while a value for the at least one biomarker found in the test sample being different is indicative for sufficient quality.

Preferably, in accordance with the aforementioned method of the present invention said reference is derived from a sample or plurality of samples known to be of insufficient quality. More preferably, in such a case an amount of the at least one biomarker in the sample being essentially identical to the said reference is indicative for insufficient quality, while an amount which differs therefrom is indicative for sufficient quality.

Also preferably, the said reference is derived from a sample or plurality of samples known to be of sufficient quality. More preferably, in such a case an amount of the at least one biomarker in the sample being essentially identical to the said reference is indicative for sufficient quality, while an amount which differs therefrom is indicative for insufficient quality.

The relative values or degrees of changes of the at least one biomarker of said individuals of the population can be determined as specified elsewhere herein. How to calculate a suitable reference value, preferably, the average or median, is well known in the art.

The value for the at least one biomarker of the test sample and the reference values are essentially identical, if the values for the characteristic features and, in the case of quantitative determination, the intensity values are essentially identical. Essentially identical means that the difference between two values is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value, preferably, the 50^{th}, 60^{th}, 70^{th}, 80^{th}, 90^{th} or 95^{th} percentile of the reference value. Statistical test for determining whether two amounts are essentially identical are well known in the art and are also described elsewhere herein.

An observed difference for two values, on the other hand, shall be statistically significant. A difference in the relative or absolute value is, preferably, significant outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference value. Preferred relative changes of the medians or degrees of changes are described in the accompanying Tables as well as in the Examples. In the Tables below, a preferred relative change for the biomarkers is indicated as “up” for an increase and “down” for a decrease in column “direction of change”. Values for preferred degrees of changes are indicated in the column “estimated fold change”. The preferred references for the aforementioned relative changes or degrees of changes are indicated in the Tables below as well. It will be understood that these changes are, preferably, observed in comparison to the references indicated in the respective Tables, below.

Preferably, the reference, i.e. values for at least one characteristic feature of the at least one biomarker or ratios thereof, will be stored in a suitable data storage medium such as a database and are, thus, also available for future assessments.

The term “comparing” refers to determining whether the determined value of a biomarker is essentially identical to a reference or differs therefrom. Preferably, a value for a biomarker is deemed to differ from a reference if the observed difference is statistically significant which can be determined by statistical techniques referred to elsewhere in this description. If the difference is not statistically significant, the biomarker value and the reference are essentially identical. Based on the comparison referred to above, the quality of a sample can be assessed, i.e. it can be assessed whether the sample is of sufficient quality, or not.

For the specific biomarkers referred to in this specification, preferred values for the changes in the relative amounts or ratios (i.e. the changes expressed as the ratios of the medians) are found in the Tables, below. Based on the ratios of the biomarkers and the calculated p-values as shown in Tables 1, 1′, 2, 2′, 3, 3′, 4, 5, 5′, 6, 7, and/or 8 below, preferably, Tables 1, 2, 3, 4, 5, 6, 7 and/or 8 below, it can be derived whether an increase or a decrease of a given biomarker is indicative for a sample of insufficient quality.

The comparison is, preferably, assisted by automation. For example, a suitable computer program comprising algorithms for the comparison of two different data sets (e.g., data sets comprising the values of the characteristic feature(s)) may be used. Such computer programs and algorithms are well known in the art. Notwithstanding the above, a comparison can also be carried out manually.

In a preferred embodiment, the biomarker or biomarkers is/are selected according to the criterion “assayability” (Tables 1a, 2a, 3a, 4a, 5a, 6a, 7a, 8a, 1a′, 2a′, 3a′, and 5′). As used in the context of biomarkers of the present invention, the term “assayability” relates to the property of a biomarker of being analyzable by at least one commercially available clinical laboratory assay, like, preferably, enzymatic, colorimetric or immunological assays.

In a further preferred embodiment, the biomarker or biomarkers is/are selected according to the criterion “performance” (Tables 1 b, 2b, 4b, 5b, 6b, 8b, and 2b′). As used in the context of biomarkers of the present invention, the term “performance” relates to the property of a biomarker having an as low as possible p-value.

In a further preferred embodiment, the biomarker or biomarkers is/are selected according to the criterion “GC-polar” (Tables 1c, 2c, 3c, 4c, 5c, 6c, 7c, 8c, 1c′, 2c′, 3c′, and 5′). As used in the context of biomarkers of the present invention, the term “GC-polar” relates to the property of a biomarker of being analyzable from the polar fraction, preferably obtained as described in the examples herein below, by a gas chromatographic method.

In a further preferred embodiment, the biomarker or biomarkers is/are selected according to the criterion “uniqueness” (Table 9). As used in the context of biomarkers of the present invention, the term “uniqueness” relates to the property of a biomarker of specifically indicating a specific pre-analytical confounding factor (quality issue). Thus, preferably, by determining a biomarker of Table 9 in a sample, it can be determined whether said sample was compromised by the quality issue indicated in said Table. It is understood by the skilled person that the direction of change of a specific biomarker can be read from the Table referenced in Table 9.

Advantageously, it has been found in the study underlying the present invention that the amounts of the specific biomarkers referred to above are indicators for the quality of a sample of biological material with respect to various pre-analytical confounding factors of relevance, such as improper processing and storage, hemolysis, contamination with blood cells, microclotting of blood samples destined for plasma preparation and other pre-analytical steps. Accordingly, the at least one biomarker as specified above in a sample can, in principle, be used for assessing whether a sample is of sufficient quality for metabolomics analysis, or not. This is particularly helpful for an efficient metabolomic diagnosis of diseases or medical conditions where proper sample quality is decisive for a reliable diagnosis.

The definitions and explanations of the terms made above apply mutatis mutandis for the following embodiments of the present invention except specified otherwise herein below.

In a preferred embodiment of the method of the invention, the biological sample is assessed for or further assessed for prolonged processing of plasma and wherein said at least one biomarker is from Table 1 or 1′, preferably Table 1. In a preferred embodiment, the marker is from Table 1a, 1b, 1c, 1a′, and/or 1c′.

In another preferred embodiment of the method of the invention, the biological sample is assessed for or further assessed for prolonged processing of blood and wherein said at least one biomarker is from Table 2 or 2′, preferably Table 2. In a preferred embodiment, the marker is from Table 2a, 2b, 2c, 2a′, 2b′, and/or 2c′.

In a further preferred embodiment of the method of the invention, the biological sample is assessed for or further assessed for hemolysis and wherein said at least one biomarker is from Table 3 or 3′, preferably Table 3. In a preferred embodiment, the marker is from Table 3a, 3c, 3a′, and/or 3c′.

In yet a preferred embodiment of the method of the present invention, the biological sample is assessed for or further assessed for microclotting and wherein said at least one biomarker is from Table 4 or 4′, preferably Table 4. In a preferred embodiment, the marker is from Table 4a, 4b, and/or 4c.

In a furthermore preferred embodiment of the method of the present invention, the biological sample is assessed for or further assessed for contamination with blood cells and wherein said at least one biomarker is from Table 5 or 5′, preferably Table 5. In a preferred embodiment, the marker is from Table 5a, 5b, and/or 5c. In a preferred embodiment, the aforesaid blood cells are white blood cells.

Moreover, in a preferred embodiment of the method of the present invention, the biological sample is assessed for or further assessed for improper storage and wherein said at least one biomarker is from Table 6 or 6′, preferably Table 6. In a preferred embodiment, the marker is from Table 6a, 6b, and/or 6c.

Moreover, in a preferred embodiment of the method of the present invention, the biological sample is assessed for or further assessed for improper freezing and wherein said at least one biomarker is from Table 7 or 7′, preferably Table 7. In a preferred embodiment, the marker is from Table 7a and/or 7c.

Moreover, in a preferred embodiment of the method of the present invention, the biological sample is assessed for prolonged coagulation of blood and wherein said at least one biomarker is from Table 8 or 8′, preferably Table 8. In a preferred embodiment, the marker is from Table 8a, 8b, and/or 8c.

Thus, in preferred embodiments of the method of the present invention, the biological material may be assessed for any one of the aforementioned confounding factors individually or a combination of confounding factors selected from the group consisting of: prolonged processing of plasma, prolonged processing of blood, hemolysis, microclotting, contamination with blood cells improper storage, improper freezing, and prolonged coagulation of blood. Preferred combinations may be, for example:
prolonged processing of plasma and prolonged processing of blood;
prolonged processing of plasma, prolonged processing of blood, and hemolysis;
prolonged processing of plasma, prolonged processing of blood, hemolysis, and microclotting;
prolonged processing of plasma, prolonged processing of blood, hemolysis, microclotting, and contamination with blood cells;
prolonged processing of plasma, prolonged processing of blood, hemolysis, microclotting, contamination with blood cells and improper storage
prolonged processing of plasma, prolonged processing of blood, hemolysis, microclotting, contamination with blood cells, improper storage and improper freezing
   prolonged processing of plasma, prolonged processing of blood, hemolysis, microclotting, contamination with blood cells, improper storage, improper freezing, and prolonged coagulation of blood
prolonged processing of blood, and hemolysis;
prolonged processing of blood, hemolysis, and microclotting;
prolonged processing of blood, hemolysis, microclotting, and contamination with blood cells;
prolonged processing of blood, hemolysis, microclotting, contamination with blood cells and improper storage
prolonged processing of blood, hemolysis, microclotting, contamination with blood cells, improper storage and improper freezing
prolonged processing of blood, hemolysis, microclotting, contamination with blood cells, improper storage, improper freezing, and prolonged coagulation of blood
prolonged processing of plasma, and hemolysis;
prolonged processing of plasma, hemolysis, and microclotting;
prolonged processing of plasma, hemolysis, microclotting, and -contamination with blood cells;
prolonged processing of plasma, hemolysis, microclotting, contamination with blood cells and improper storage
prolonged processing of plasma, hemolysis, microclotting, contamination with blood cells, improper storage, and prolonged coagulation of blood
prolonged processing of plasma, prolonged processing of blood, and microclotting;
prolonged processing of plasma, prolonged processing of blood, microclotting, and contamination with blood cells;
prolonged processing of plasma, prolonged processing of blood, microclotting, contamination with blood cells and improper storage
prolonged processing of plasma, prolonged processing of blood, microclotting, contamination with blood cells, improper storage and improper freezing
prolonged processing of plasma, prolonged processing of blood, microclotting, contamination with blood cells, improper storage, improper freezing, and prolonged coagulation of blood
prolonged processing of plasma, prolonged processing of blood, hemolysis, and contamination with blood cells;
prolonged processing of plasma, prolonged processing of blood, hemolysis, contamination with blood cells and improper storage
prolonged processing of plasma, prolonged processing of blood, hemolysis, contamination with blood cells, improper storage and improper freezing
prolonged processing of plasma, prolonged processing of blood, hemolysis, contamination with blood cells, improper storage, improper freezing, and prolonged coagulation of blood
prolonged processing of plasma, prolonged processing of blood, hemolysis, microclotting, and improper storage
prolonged processing of plasma, prolonged processing of blood, hemolysis, microclotting, improper storage and improper freezing
prolonged processing of plasma, prolonged processing of blood, hemolysis, microclotting, improper storage, improper freezing, and prolonged coagulation of blood
prolonged processing of plasma, prolonged processing of blood, and hemolysis;
prolonged processing of plasma, prolonged processing of blood, and improper storage
prolonged processing of plasma, prolonged processing of blood, improper storage, and prolonged coagulation of blood

The present invention also relates to a device or system for assessing the quality of a biological sample comprising:
a) an analyzing unit for the said sample comprising a detector for at least one biomarker of Tables 1, 1′, 2, 2′, 3, 3′, 4, 5, 5′, 6, 7, and/or 8, preferably Tables 1, 2, 3, 4, 5, 6, 7 and/or 8, said detector allowing for the determination of the amount of the said at least one biomarker in the sample; and operatively linked thereto,
(b) an evaluation unit comprising a data processing unit and a data base, said data base comprising a stored reference and said data processing unit having tangibly embedded an algorithm for carrying out a comparison of the amount of the at least one biomarker determined by the analyzing unit and the stored reference and for generating an output information based on which the assessment of the quality is established.

A device as used herein shall comprise at least the aforementioned units. The units of the device are operatively linked to each other. How to link the means in an operating manner will depend on the type of units included into the device. For example, where the detector allows for automatic qualitative or quantitative determination of the biomarker, the data obtained by said automatically operating analyzing unit can be processed by, e.g., a computer program in order to facilitate the assessment in the evaluation unit. Preferably, the units are comprised by a single device in such a case. Said device may accordingly include an analyzing unit for the biomarker and a computer or data processing device as evaluation unit for processing the resulting data for the assessment and for stabling the output information. Preferred devices are those which can be applied without the particular knowledge of a specialized clinician, e.g., electronic devices which merely require loading with a sample. The output information of the device, preferably, is a numerical value which allows drawing conclusions on the quality of the sample and, thus, is an aid for the reliability of a diagnosis or for troubleshooting.

A preferred reference to be used as a stored reference in accordance with the device of the present invention is an amount for the at least one biomarker to be analyzed or values derived therefrom which are derived from a sample or plurality of samples of insufficient quality. In such a case, the algorithm tangibly embedded, preferably, compares the determined amount for the at least one biomarker with the reference wherein an identical or essentially identical amount or value shall be indicative for a sample of insufficient quality while an amount which differs indicates a sample of sufficient quality.

Alternatively, another preferred reference to be used as stored reference in accordance with the device of the present invention is an amount for the at least one biomarker to be analyzed or values derived therefrom which are derived from a sample or plurality of samples of sufficient quality. In such a case, the algorithm tangibly embedded, preferably, compares the determined amount for the at least one biomarker with the reference wherein an identical or essentially identical amount or value shall be indicative for a sample of sufficient quality while an amount which differs indicates a sample of insufficient quality.

Preferred differences are those indicated as relative changes or degrees of changes for the individual biomarkers in the Tables below.

Preferably, in the device of the invention, at least one biomarker of Table 1 can be used for assessing prolonged processing of plasma. Preferably, in the device of the invention, at least one biomarker of Table 2 can be used for assessing prolonged processing of blood. Preferably, in the device of the invention, at least one biomarker of Table 3 can be used for assessing hemolysis. Preferably, in the device of the invention, at least one biomarker of Table 4 can be used for assessing microclotting. Preferably, in the device of the invention, at least one biomarker of Table can be used for assessing contamination with blood cells. Preferably, in the device of the invention, at least one biomarker of Table 6 can be used for assessing improper storage. Preferably, in the device of the invention, at least one biomarker of Table 7 can be used for assessing improper freezing. Preferably, in the device of the invention, at least one biomarker of Table 8 can be used for assessing prolonged coagulation time of blood.

The units of the device, also preferably, can be implemented into a system comprising several devices which are operatively linked to each other. Depending on the units to be used for the system of the present invention, said means may be functionally linked by connecting each mean with the other by means which allow data transport in between said means, e.g., glass fiber cables, and other cables for high throughput data transport. Nevertheless, wireless data transfer between the means is also envisaged by the present invention, e.g., via LAN (Wireless LAN, W-LAN). A preferred system comprises means for determining biomarkers. Means for determining biomarkers as used herein encompass means for separating biomarkers, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, H PLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS are used in the system of the present invention as described in detail elsewhere in the specification. Further comprised shall be means for comparing and/or analyzing the results obtained from the means for determination of biomarkers. The means for comparing and/or analyzing the results may comprise at least one databases and an implemented computer program for comparison of the results. Preferred embodiments of the aforementioned systems and devices are also described in detail below.

Furthermore, the present invention relates to a data collection comprising characteristic values of at least one biomarker being indicative for sufficient or insufficient quality of a sample of biological material.

The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a ClientServer-System. More preferably, the database is structured as to allow a search algorithm to compare a test data set with the data sets comprised by the data collection. Specifically, by using such an algorithm, the database can be searched for similar or identical data sets being indicative for a sample quality as set forth above (e.g. a query search). Thus, if an identical or similar data set can be identified in the data collection, the test data set will be associated with the said quality. Consequently, the information obtained from the data collection can be used, e.g., as a reference for the methods of the present invention described above. More preferably, the data collection comprises characteristic values of all biomarkers comprised by any one of the groups recited above.

In light of the foregoing, the present invention encompasses a data storage medium comprising the aforementioned data collection.

The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising:
(a) means for comparing characteristic values of the at least one biomarker of a sample operatively linked to
(b) a data storage medium as described above.

The term “system” as used herein relates to different means which are operatively linked to each other. Said means may be implemented in a single device or may be physically separated devices which are operatively linked to each other. The means for comparing characteristic values of biomarkers, preferably, based on an algorithm for comparison as mentioned before. The data storage medium, preferably, comprises the aforementioned data collection or database, wherein each of the stored data sets being indicative for a sample quality referred to above. Thus, the system of the present invention allows identifying whether a test data set is comprised by the data collection stored in the data storage medium. Consequently, the methods of the present invention can be implemented by the system of the present invention.

In a preferred embodiment of the system, means for determining characteristic values of biomarkers of a sample are comprised. The term “means for determining characteristic values of biomarkers” preferably relates to the aforementioned devices for the determination of metabolites such as mass spectrometry devices, NMR devices or devices for carrying out chemical or biological assays for the biomarkers.

In general, the present invention contemplates the use of at least one biomarker of at least one biomarker from Tables 1, 1′, 2, 2′, 3, 3′, 4, 5, 5′, 6, 7, and/or 8, preferably Tables 1, 2, 3, 4, 5, 6, 7 and/or 8, or a detection agent therefor for assessing the quality of a sample.

Preferably, at least one biomarker of Table 1 and/or 1′ can be used for assessing prolonged processing of plasma. Preferably, at least one biomarker of Table 2 and/or 2′ can be used for assessing prolonged processing of blood. Preferably, at least one biomarker of Table 3 and/or 3′ can be used for assessing hemolysis. Preferably, at least one biomarker of Table 4 can be used for assessing microclotting. Preferably, at least one biomarker of Table 5 and/or 5′ can be used for assessing contamination with blood cells. Preferably, at least one biomarker of Table 6 can be used for assessing improper storage. Preferably, at least one biomarker of Table 7 can be used for assessing improper freezing. Preferably, at least one biomarker of Table 8 can be used for assessing prolonged coagulation time of blood.

More preferably, at least one biomarker of Table 1 can be used for assessing prolonged processing of plasma. More preferably, at least one biomarker of Table 2 can be used for assessing prolonged processing of blood. More preferably, at least one biomarker of Table 3 can be used for assessing hemolysis. More preferably, at least one biomarker of Table 5 can be used for assessing contamination with blood cells.

How detection agents can be manufactured based on the at least one biomarker is well known to those skilled in the art. For example, antibodies or aptameres which specifically bind to the at least one biomarker can be produced. Similarly, the biomarkers itself may be used as such compositions, e.g., within complexes or in modified or derivatized form, e.g., when analysed by GCMS.

The present invention also provides a kit for assessing the quality of a biological sample comprising a detection agent for at least one biomarker from Tables 1, 1′, 2, 2′, 3, 3′, 4, 5, 5′, 6, 7, and/or 8, preferably Tables 1, 2, 3, 4, 5, 6, 7 and/or 8, and, preferably, a reference for the said at least one biomarker.

The term “kit” as used herein refers to a collection of the aforementioned components, preferably, provided in separately or within a single container. The container also comprises instructions for carrying out the method of the present invention. These instructions may be in the form of a manual or may be provided by a computer program code which is capable of carrying out the comparisons referred to in the methods of the present invention and to establish a quality assessment of a sample when implemented on a computer or a data processing device. The computer program code may be provided on a data storage medium or device such as an optical storage medium (e.g., a Compact Disc) or directly on a computer or data processing device. Further, the kit shall comprise at least one standard for a reference as defined herein above, i.e. a solution with a pre-defined amount for the at least one biomarker representing a reference amount. Such a standard may represent, e.g., the amount of the at least one biomarker from a sample or plurality of samples of sufficient or insufficient quality.

Preferably, the kit of the present invention comprises a detection agent for at least one biomarker from each of Tables 1, 1′, 2, 2′, 3, 3′, 4, 5, 5′, 6, 7, and/or 8, preferably Tables 1, 2, 3, 4, 5, 6, 7 and/or 8, and, preferably, a reference for each of the said at least one biomarker in order to allow for assessing a sample for insufficient quality relating to any one of prolonged processing of plasma, prolonged processing of blood, hemolysis, microclotting, contamination with blood cells, improper storage and improper freezing.

In some embodiments, the kit may comprise additional components such as buffers, reagents (for example, conjugate and/or substrate), and the like, as disclosed herein.

It will be understood that the present invention also relates to the use of the kit of the invention for the aforementioned purposes of assessing sufficient or insufficient quality of a sample.

Preferably, a kit comprising at least one biomarker of Table 1 and/or 1′ can be used for assessing prolonged processing of plasma. Preferably, a kit comprising at least one biomarker of Table 2 and/or 2′ can be used for assessing prolonged processing of blood. Preferably, a kit comprising at least one biomarker of Table 3 and/or 3′ can be used for assessing hemolysis. Preferably, a kit comprising at least one biomarker of Table 4 can be used for assessing microclotting. Preferably, a kit comprising at least one biomarker of Table 5 and/or 5′ can be used for assessing contamination with blood cells. Preferably, a kit comprising at least one biomarker of Table 6 can be used for assessing improper storage. Preferably, a kit comprising at least one biomarker of Table 7 can be used for assessing improper freezing. Preferably, a kit comprising at least one biomarker of Table 8 can be used for assessing prolonged coagulation time of blood.

More preferably, a kit comprising at least one biomarker of Table 1 can be used for assessing prolonged processing of plasma. More preferably, a kit comprising at least one biomarker of Table 2 can be used for assessing prolonged processing of blood. More preferably, a kit comprising at least one biomarker of Table 3 can be used for assessing hemolysis. More preferably, a kit comprising at least one biomarker of Table 5 can be used for assessing contamination with blood cells.

In a preferred embodiment, the present invention relates to a method of performing metabolome analysis, comprising assessing the quality of at least one biological sample according to a method of the present invention, and performing metabolome analysis, preferably using only biological samples for which sufficient quality was assessed.

In a further preferred embodiment, the present invention relates to a method of performing metabolome analysis, comprising ordering an assessment of the quality of at least one biological sample according to one of the methods of the present invention, and performing metabolome analysis, preferably using only biological samples for which sufficient quality was assessed.

In a further preferred embodiment, the present invention relates to a method of stratifying biological samples according to quality, comprising assessing the quality of at least one biological sample according to a method of the present invention, and stratifying said at least one sample according to quality.

In a further preferred embodiment, the present invention relates to a method of stratifying biological samples according to quality, comprising ordering an assessment of the quality of at least one biological sample according to one of the methods of the present invention, and stratifying said at least one sample according to quality.

In a further preferred embodiment, the present invention relates to a method of removing biological samples not conforming to quality criteria from a pool of biological samples, comprising assessing the quality of at least one biological sample from said pool according to a method of the present invention, and removing said sample from said pool in case insufficient quality is assessed.

In a further preferred embodiment, the present invention relates to a method of removing biological samples not conforming to quality criteria from a pool of biological samples, comprising ordering an assessment of the quality of at least one biological sample from said pool according to a method of the present invention, and removing said sample from said pool in case insufficient quality is assessed.

In a further preferred embodiment, the present invention relates to a method of including a biological sample in a study, preferably a clinical study, comprising assessing the quality of at least one biological sample according to a method of the present invention, and including said biological sample in said study if sufficient quality is assessed.

In a further preferred embodiment, the present invention relates to a method of including a biological sample in a study, preferably a clinical study, comprising ordering an assessment of the quality of at least one biological sample according to a method of the present invention, and including said biological sample in said study if sufficient quality is assessed.

All references cited herein are herewith incorporated by reference with respect to their disclosure content in general or with respect to the specific disclosure contents indicated above.

The invention will now be illustrated by the following Examples which are not intended to restrict or limit the scope of this invention.

### EXAMPLES

### Example 1

### Experimental Design Analysing Metabolic Effects of Processing Time and Processing Temperature on Human Blood Plasma

This experiment was designed to analyse the effects of short-term incubation during pre-analytical sample processing on the human plasma metabolome in order to identify biomarkers for quality control of blood plasma biobank specimen. An EDTA plasma pool was divided into 1-ml-aliquots and these were incubated at temperatures of 4° C., 12° C. and 21° C. At the time points 0 h, 0.5 h, 2 h, 5 h and 16 h, each 10 aliquots were frozen at −80° C. and analysed as described in example 4 (sphingolipids were not analysed in Example 1). Plasma samples were analyzed in randomized analytical sequence design. The raw peak data was normalized to the median of all samples per analytical sequence to account for process variability (so called “ratios”). In order to allow an experiment-comprehensive alignment of semi-quantitative data, MxPool™ was analyzed with 12 replicated samples in the experiment and the ratios further normalized to the median of the MxPool™ samples, i.e. ratios from this studies are on the same level and therefore comparable to data from other projects that are normalized to other aliquots of the same MxPool™. Total quantified data from targeted methods (eicosanoids, catecholamines) remain with their absolute quantification data. Data was log 10 transformed to approach a normal distribution. Statistical analysis was done by a simple linear model (ANOVA) with the fixed effects “time” and “temperature”. The ANOVA factor “time” was set to the reference “0” as factor and “temperature” was set to the reference “4° C.”. Significance level was set to an alpha-error of 5%. Metabolites identified by this approach are indicators of quality diminishing effects related to increased processing time or processing temperature of biobank specimen (Table 1).

### Example 2

### Experimental Design Analysing Metabolic Effects of Different Blood Processing Procedures on Human Blood Plasma

This experiment was designed to analyse the effects of different blood sample handling procedures on the human plasma metabolom in order to identify biomarkers for quality control of blood plasma biobank specimen. Different groups of blood handling comprised the following procedures:
Beginning coagulation of blood
Prolonged incubation at 0° C.
Prolonged incubation at room temperature
Hemolysis
Contamination with white blood cells
Freezing protocol

Twenty healthy volunteers (13 females, 7 males) were recruited and 64 ml of blood were withdrawn by venous puncture using a gauge-20 safety-fly blood collection system into 3 9-ml-K₃EDTA monovettes followed by 1 ml into a neutral monovette (sample was discarded) followed by a 9-ml-neutral monovette followed by 3 9-ml-K₃EDTA monovettes. The monovettes were gently mixed by inverting to prevent hemolysis. The K₃EDTA monovettes were opened and pooled within each subject.

The blood of each subject was processed within the different groups as follows:

### Beginning Coagulation of Blood

After 5 min at room temperature, the blood from the 9-ml neutral monovette was decanted into a 9-ml-K₃EDTA monovette and the plasma prepared by centrifugation at 1500×g for 15 minutes in a refrigerated centrifuge. The plasma was frozen in liquid nitrogen and stored at −80° C. until analysis.

### Prolonged Incubation at 0° C.

2×5 ml of the blood pool was incubated at 0° C. for 4 h and 6 h, respectively. After that time period, the plasma was prepared by centrifugation at 1500×g for 15 minutes in a refrigerated centrifuge. The plasma was stored at −80° C. until analysis.

### Prolonged Incubation at Room Temperature

5 ml of the blood pool were incubated at room temperature for 1 h. After that time period, the plasma was prepared by centrifugation at 1500×g for 15 minutes in a refrigerated centrifuge. The plasma was stored at −80° C. until analysis.

### Hemolysis

2×6 ml of the blood pool were passed through a syringe with a gauge-25 (grade 1 hemolysis) and gauge-27 needle (grade 2 hemolysis), respectively. The plasma was prepared by centrifugation at 1500×g for 15 minutes in a refrigerated centrifuge. The plasma was stored at −80° C. until analysis.

Contamination with White Blood Cells/Freezing Protocol/Control

The remaining blood pool was centrifuged at 1500×g for 15 minutes in a refrigerated centrifuge. The upper plasma supernatant was withdrawn and mixed in a centrifugation tube. Aliquots of this plasma sample were frozen and stored at −80° C. until analysis to serve as control. Further aliquots of this plasma sample were frozen at −20° C. and at the end of the day transferred and stored at −80° C. until analysis (“slow freezing”—see Table 7). The lower plasma supernatant was mixed with material from the buffy layer of the centrifugation tube resulting in two grades of contamination with white blood cells.

The plasma samples of this experiment were analysed as described in example 4 in randomized analytical sequence design. Metabolite profiling provides a semi-quantitative analytical platform resulting in relative metabolite level to a defined reference group (“ratio”). To support this concept and also to allow an alignment of different analytical batches (“experiments”), two different reference sample types were run in parallel throughout the whole process. First, a project pool was generated from aliquots of all samples and measured with 4 replicates within each analytical sequence. For all semi-quantitatively analyzed metabolites, the data were normalized against the median in the pool reference samples within each analytical sequence to give pool-normalized ratios (performed for each sample per metabolite). This compensated for inter- and intra-instrumental variation. Second, MxPool™ was analyzed with 12 replicated samples in the experiment and the pool-normalized ratios further normalized to the median of the MxPool™ samples, i.e. ratios from this studies are on the same level and therefore comparable to data from other projects that are normalized to other aliquots of the same MxPool™. Total quantified data from targeted methods (eicosanoids, catecholamines) remain with their absolute quantification data.

### Data Analysis:

Data was log 10 transformed prior to statistical analysis in order to approach a normal distribution. Metabolite ratio changes were calculated by a mixed linear model (ANOVA) with subject as random intercept and gender as fixed effect. Ratios in Tables 2-5 are expressed relative to the control group.

### Example 3

### Experimental Design Analysing Metabolic Effects of Long-Term Storage at −20° C. On Human Blood Plasma

This experiment was designed to analyse the effects of prolonged storage at −20° C. on the human plasma metabolome in order to identify biomarkers for quality control of blood plasma biobank specimen. Aliquots of an EDTA plasma pool were frozen at −20° C. or in liquid nitrogen, respectively. After 181 days and 365 days, 4 aliquots of samples stored at each temperature were analysed by metabolite profiling as described in example 4 (sphingolipids were not analysed in Example 3). Plasma samples were analyzed in randomized analytical sequence design. A project pool was generated from aliquots of all samples and measured with 4 replicates within each analytical sequence. The raw peak data was normalized to the median of the project pool per analytical sequence to account for process variability (so called “ratios”). Ratios were log 10 transformed to approach a normal distribution of data. Statistical analysis of metabolite changes after storage at −20° C. for 181 days and 365 days relative to storage in liquid nitrogen for the same time period was done by a simple linear model (ANOVA) with the fixed effect “temperature” set to a reference of “−196° C.”. Significance level was set to an alpha-error of 5%. Metabolites are biomarkers indicating quality issues in biobank specimen that are related to increased plasma storage time or temperature (Table 6).

### Example 4

### Sample Preparation for MS Analysis and MS Analysis

Human plasma samples were prepared and subjected to LC-MS/MS and GC-MS or SPE-LC-MS/MS (hormones) analysis as described in the following. Proteins were separated by precipitation from blood plasma. After addition of water and a mixture of ethanol and dichlormethan the remaining sample was fractioned into an aqueous, polar phase and an organic, lipophilic phase.

For the transmethanolysis of the lipid extracts a mixture of 140 μl of chloroform, 37 μl of hydrochloric acid (37% by weight HCl in water), 320 μl of methanol and 20 μl of toluene was added to the evaporated extract. The vessel was sealed tightly and heated for 2 hours at 100° C., with shaking. The solution was subsequently evaporated to dryness. The residue was dried completely.

The methoximation of the carbonyl groups was carried out by reaction with methoxyamine hydrochloride (20 mg/ml in pyridine, 100 l for 1.5 hours at 60° C.) in a tightly sealed vessel. 20 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 100 μl of N-methyl-N-(trimethylsilyl)-2,2,2-trifluoroacetamide (MSTFA) was carried out for 30 minutes at 60° C., again in the tightly sealed vessel. The final volume before injection into the GC was 220 μl.

For the polar phase the derivatization was performed in the following way: The methoximation of the carbonyl groups was carried out by reaction with methoxyamine hydrochloride (20 mg/ml in pyridine, 50 l for 1.5 hours at 60° C.) in a tightly sealed vessel. 10 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 50 μl of N-methyl-N-(trimethylsilyl)-2,2,2-trifluoroacetamide (MSTFA) was carried out for 30 minutes at 60° C., again in the tightly sealed vessel. The final volume before injection into the GC was 110 μl.

The GC-MS systems consist of an Agilent 6890 GC coupled to an Agilent 5973 MSD. The autosamplers are CompiPal or GCPal from CTC.

For the analysis usual commercial capillary separation columns (30 m×0.25 mm×0.25 μm) with different poly-methyl-siloxane stationary phases containing 0% up to 35% of aromatic moieties, depending on the analysed sample materials and fractions from the phase separation step, were used (for example: DB-1 ms, HP-5 ms, DB-XLB, DB-35 ms, Agilent Technologies). Up to 1 μL of the final volume was injected splitless and the oven temperature program was started at 70° C. and ended at 340° C. with different heating rates depending on the sample material and fraction from the phase separation step in order to achieve a sufficient chromatographic separation and number of scans within each analyte peak. Furthermore RTL (Retention Time Locking, Agilent Technologies) was used for the analysis and usual GC-MS standard conditions, for example constant flow with nominal 1 to 1.7 ml/min. and helium as the mobile phase gas, ionisation was done by electron impact with 70 eV, scanning within a m/z range from 15 to 600 with scan rates from 2.5 to 3 scans/sec and standard tune conditions.

The HPLC-MS systems consisted of an Agilent 1100 LC system (Agilent Technologies, Waldbronn, Germany) coupled with an API 4000 Mass spectrometer (Applied Biosystem/MDS SCIEX, Toronto, Canada). HPLC analysis was performed on commercially available reversed phase separation columns with C18 stationary phases (for example: GROM ODS 7 pH, Thermo Betasil C18). Up to 10 μL of the final sample volume of evaporated and reconstituted polar and lipophilic phase was injected and separation was performed with gradient elution using methanol/water/formic acid or acetonitrile/water/formic acid gradients at a flowrate of 200 μL/min.

Mass spectrometry was carried out by electrospray ionisation in positive mode for the non-polar fraction and negative or positive mode for the polar fraction using multiple-reaction-monitoring(MRM)-mode and fullscan from 100-1000 amu.

### Analysis of Steroids and Catecholamines in Plasma Samples:

Steroids and their metabolites were measured by online SPE-LC-MS (Solid phase extraction-LC-MS). Catecholamines and their metabolites were measured by online SPE-LC-MS as described by Yamada et al. (Yamada H, Yamahara A, Yasuda S, Abe M, Oguri K, Fukushima S, Ikeda-Wada S: Dansyl chloride derivatization of methamphetamine: a methode with advantages for screening and analysis of methamphetamine in urine. Journal of Analytical Toxicology, 26(1): 17-22 (2002)).

### Analysis of Eicosanoids in Plasma Samples

Eicosanoids and related were measured out of plasma by offline- and online-SPE LC-MS/MS (Solid phase extraction-LC-MS/MS) (Masoodi M and Nicolaou A: Rapid Commun Mass Spectrom. 2006; 20(20): 3023-3029. Absolute quantification was performed by means of stable isotope-labelled standards.

### Example 5

### Experimental Design Analysing Metabolic Effects of Increased Coagulation Time of Blood

This experiment describes the analysis of effects of increased coagulation time of blood on the human serum metabolome in order to identify biomarkers for quality control of blood serum biobank specimen. 145 blood samples were allowed to clot at room temperature for 1-2 h. Another group of 46 blood samples were allowed to clot for 24 h at room temperature. The clotted samples were centrifuged and the serum supernatants were removed and frozen. Serum samples were stored at −80° C. previous to metabolite profiling analysis as described in Example 4 (sphingolipids were not analysed in Example 5). The serum samples of this experiment were analysed in a randomized analytical sequence design. A pool was generated from aliquots of all samples and measured with 4 replicates within each analytical sequence. For all semi-quantitatively analyzed metabolites, the data were normalized against the median in the pool reference samples within each analytical sequence to give pool-normalized ratios (performed for each sample per metabolite). This compensated for inter- and intra-instrumental variation.

### Data Analysis:

Data was log 10 transformed prior to statistical analysis in order to approach a normal distribution. Metabolite ratio changes were calculated by a simple linear model (ANOVA) with the processing group and gender as fixed effects. Data in Table 8 is expressed as ratios and p-values of 24-h-blood clotting period of blood relative to direct processing of blood to serum.

TABLE 1
List of identified biomarkers indicating quality issue in plasma samples related
to increased processing time of plasma samples. Relative ratios of samples processed at
different temperatures (4° C., 12° C., 21° C.) and times
(0.5 h, 2 h, 5 h, 16 h) compared to control samples (upper part of table 1) as
well as corresponding p-values (lower part of table 1) are given.

| | | | | | | |
|---|---|---|---|---|---|---|
| Temp. ° C. | 4 | 4 | 4 | 4 | 12 | 12 |
| Time | 0.5 | 2 | 5 | 16 | 0.5 | 2 |

| | | |
|---|---|---|
| | | Ratio relative |
| Biomarker (Metabolite) | Ratio relative to = 0 at 4° c. | to = 0 at 12° C. |

upper part:

| | | | | | | |
|---|---|---|---|---|---|---|
| 3,4-Dihydroxyphenylacetic | 1.0625 | 0.9364 | 0.8259 | 0.4887 | 1.0282 | 0.9773 |
| acid (DOPAC) | | | | | | |
| 5-Hydroxyeicosatetraenoic | 0.9892 | 1.098 | 1.1755 | 1.868 | 1.0111 | 1.1428 |
| acid | | | | | | |
| (C20:trans[6]cis[8,11,14]4) | | | | | | |
| (5-HETE) | | | | | | |
| 12- | 0.9311 | 1.1504 | 1.3211 | 2.1302 | 0.8739 | 1.4264 |
| Hydroxyeicosatetraenoic | | | | | | |
| acid (C20:cis[5,8,10,14]4) | | | | | | |
| Glutamate | 1.041 | 1.1561 | 1.2597 | 1.1884 | 1.0246 | 1.1235 |
| 15- | 0.9722 | 1.042 | 1.1046 | 1.3129 | 0.9529 | 1.0739 |
| Hydroxyeicosatetraenoic | | | | | | |
| acid (C20:cis[5,8,11,13]4) | | | | | | |
| 3,4-Dihydroxyphenylglycol | 1.0225 | 0.9546 | 0.9674 | 0.824 | 1.0235 | 1.0218 |
| (DOPEG) | | | | | | |
| 11- | 0.955 | 1.0678 | 1.2197 | 1.5519 | 0.9638 | 1.1647 |
| Hydroxyeicosatetraenoic | | | | | | |
| acid (C20:cis[5,8,12,14]4) | | | | | | |
| 3,4- | 1.0125 | 0.9554 | 1.0138 | 0.8497 | 0.9905 | 0.9758 |
| Dihydroxyphenylalanine | | | | | | |
| (DOPA) | | | | | | |
| 8-Hydroxyeicosatetraenoic | 1.0176 | 1.0558 | 1.0581 | 1.1872 | 1.0143 | 1.0797 |
| acid | | | | | | |
| (C20:trans[5]cis[9,11,14]4) | | | | | | |
| (8-HETE) | | | | | | |
| Prostaglandin D2 | 1.1073 | 1.3245 | 1.3322 | 1.5213 | 1.0169 | 1.5017 |
| Maltose | 1.0104 | 1.1938 | 1.1892 | 1.7419 | 1.1533 | 1.2714 |
| alpha-Ketoglutarate | 1.0259 | 1.0847 | 0.9542 | 1.0734 | 1.1347 | 1.0456 |
| Noradrenaline (Norepinephrine) | 1.0347 | 0.9328 | 0.8868 | 0.8226 | 1.0203 | 1.0804 |
| Cysteine | 1.001 | 0.9417 | 0.8919 | 0.7964 | 1.0123 | 0.9543 |
| Glutamate to glutamine | 0.9709 | 1.3168 | 1.457 | 1.1255 | 1.2391 | 1.4551 |
| intra-sample ratio | | | | | | |
| Glycerate | 0.9436 | 1.081 | 1.2446 | 1.6318 | 1.0087 | 1.1107 |
| 8,9-Dihydroxyeicosatrienoic | 1.0118 | 1.0702 | 1.0511 | 1.2078 | 1.0603 | 1.0368 |
| acid (C20:cis[5,11,14]3) | | | | | | |
| Threonic acid | 1.1588 | 1.3421 | 1.5022 | 1.7422 | 1.2672 | 1.5643 |
| delta-12-Prostaglandin D2 | 1.1939 | 1.4691 | 1.3384 | 4.4524 | 0.9452 | 1.7445 |
| Prostaglandin E2 | 1.3299 | 1.677 | 1.8657 | 2.5466 | 1.1782 | 1.7292 |
| Glycerol-3-phosphate, polar | 1.1144 | 1.1108 | 1.0752 | 1.2142 | 0.9725 | 0.9103 |
| fraction | | | | | | |
| Lysophosphatidylcholine | 1.116 | 1.0932 | 1.102 | 1.1825 | 1.0263 | 0.9893 |
| (C17:0) | | | | | | |
| Pyruvate | 0.994 | 0.9989 | 0.969 | 0.9556 | 0.9869 | 1.0088 |
| 12- | 0.9653 | 1.0417 | 0.9699 | 0.9246 | 0.916 | 1.054 |
| Hydroxyheptadecatrienoic | | | | | | |
| acid (C17:[5,8,10]3) | | | | | | |
| 13- | 0.9662 | 0.9894 | 1.0255 | 1.004 | 0.9807 | 1.028 |
| Hydroxyoctadecadienoic | | | | | | |
| acid (13-HODE) | | | | | | |
| (C18:cis[9]trans[11]2) | | | | | | |
| Glutamine | 0.9927 | 0.974 | 0.964 | 0.9903 | 0.9641 | 0.9387 |
| Adrenaline (Epinephrine) | 1.0335 | 0.9416 | 0.9691 | 0.8541 | 1.0099 | 1.0043 |
| 3-Phosphoglycerate (3- | 0.9815 | 1.0468 | 1.1889 | 1.5499 | 1.1887 | 1.0339 |
| PGA) | | | | | | |
| Lysophosphatidylcholine | 0.9547 | 0.9734 | 0.9783 | 0.9684 | 1.0283 | 1.0184 |
| (C18:0) | | | | | | |
| Thromboxane B2 | 0.9789 | 1.0456 | 0.95 | 0.9366 | 0.8474 | 1.1383 |
| 9-Hydroxyoctadecadienoic | 0.9846 | 1.0033 | 1.0146 | 0.9973 | 0.9684 | 1.0188 |
| acid (9-HODE) | | | | | | |
| (C18:trans[10]cis[12]2) | | | | | | |
| Cystine | 0.9964 | 0.8788 | 0.9507 | 0.7651 | 1.0256 | 0.9383 |
| Phosphatidylcholine | 0.998 | 1.0144 | 1.0125 | 1.0273 | 1.0051 | 0.9915 |
| (C16:1,C18:2) | | | | | | |
| Alanine | 0.9997 | 1.0012 | 1.0049 | 1.0011 | 0.9978 | 0.9967 |
| Glycerol, polar fraction | 0.9728 | 0.9908 | 1.0205 | 0.9938 | 0.9865 | 1.0172 |
| Isocitrate | 1.0281 | 1.071 | 1.0728 | 1.1567 | 0.9849 | 1.0352 |
| Lysophosphatidylcholine | 0.9719 | 1.0251 | 0.9895 | 1.1072 | 0.9699 | 1.0912 |
| (C20:4) | | | | | | |
| 1-Hydroxy-2-amino- | 1.0587 | 1.0785 | 1.0512 | 1.0455 | 1.0633 | 1.0463 |
| (cis.trans)-3,5- | | | | | | |
| octadecadiene (from | | | | | | |
| sphingolipids) | | | | | | |
| Lysophosphatidylcholine | 0.9734 | 0.9951 | 0.9986 | 1.0216 | 0.9788 | 0.9858 |
| (C18:1) | | | | | | |
| Ceramide (d18:1,C24:0) | 1.0451 | 1.064 | 1.0437 | 1.1264 | 1.0957 | 0.9797 |
| 14.15- | 1.0548 | 1.0641 | 1.043 | 1.1126 | 1.0341 | 0.9657 |
| Dihydroxyeicosatrienoic | | | | | | |
| acid (C20:cis[5,8,11]3) | | | | | | |
| Lysophosphatidylcholine | 0.8507 | 0.9236 | 0.9256 | 1.0063 | 0.9587 | 0.9152 |
| (C18:2) | | | | | | |
| erythro-Dihydrosphingosine | 1.1127 | 1.1355 | 1.1208 | 1.1276 | 1.0831 | 1.074 |
| (d16:0) | | | | | | |
| Valine | 0.9955 | 0.9933 | 0.9922 | 0.9932 | 1.0062 | 0.9957 |
| erythro-Sphingosine | 1.0865 | 1.1217 | 1.1009 | 1.1068 | 1.0635 | 1.0576 |
| (d18:1) | | | | | | |
| Creatine | 1.048 | 1.0926 | 1.0425 | 1.0404 | 1.018 | 1.0181 |
| myo-Inositol-2-phosphate. | 0.9573 | 0.8559 | 0.9323 | 0.8765 | 1.2195 | 1.181 |
| lipid fraction (myo- | | | | | | |
| Inositolphospholipids) | | | | | | |
| Leucine | 0.9956 | 0.9999 | 0.9989 | 0.9971 | 1.0007 | 1.0024 |
| Quinic acid | 1.0086 | 1.0513 | 1.0274 | 1.0107 | 1.0528 | 1.0278 |
| Glycerol, lipid fraction | 0.9526 | 1.0041 | 0.9633 | 0.9595 | 1.0251 | 1.0181 |
| Lysophosphatidylcholine | 1.0009 | 1.0562 | 1.1208 | 1.2595 | 1.0084 | 1.0847 |
| (C16:0) | | | | | | |
| Eicosanoic acid (C20:0) | 0.9592 | 1.0036 | 0.9635 | 0.9832 | 0.9257 | 0.9507 |
| Octadecanoylcarnitine | 0.9602 | 1.0127 | 0.9551 | 1.0335 | 0.985 | 0.9434 |
| Phosphatidylcholine | 1.0043 | 1.0146 | 1.0047 | 1.0136 | 0.9969 | 0.9932 |
| (C18:0.C18:1) | | | | | | |
| Serine | 1.0063 | 1.0301 | 1.008 | 1.0206 | 1.0164 | 1.005 |
| Erythrol | 1.0124 | 0.9162 | 0.9604 | 1.0089 | 1.0027 | 1.011 |
| Phosphatidylcholine | 0.9658 | 0.9739 | 0.9862 | 0.9967 | 1.0035 | 0.9863 |
| (C16:0.C16:0) | | | | | | |
| Glucose-6-phosphate | 1.1111 | 0.9894 | 1.2034 | 1.2798 | 0.914 | 1.0705 |
| Cholesta-2,4,6-triene | 0.892 | 0.9358 | 0.9062 | 0.9107 | 0.876 | 0.8604 |
| trans-4-Hydroxyproline | 1 | 1.0023 | 1.0257 | 0.9963 | 1.0103 | 1.0156 |
| Cholesterylester C18:2 | 0.9681 | 0.99 | 0.9805 | 1.0078 | 0.9956 | 0.9907 |
| Docosahexaenoic acid | 0.9906 | 0.9946 | 1.0033 | 1.0099 | 1.0092 | 1.0134 |
| (C22:cis[4,7,10,13,16,19]6) | | | | | | |
| 4-Hydroxysphinganine | 1.0107 | 0.9966 | 1.0109 | 0.9903 | 1.0781 | 1.0962 |
| (t18:0, Phytosphingosine), | | | | | | |
| total | | | | | | |
| 14-Methylhexadecanoic | 1.023 | 1.01 | 1.0295 | 0.9809 | 1.0133 | 1.0066 |
| acid | | | | | | |
| TAG(C16:0,C18:1,C18:2) | 1.0406 | 1.0271 | 0.9911 | 1.0322 | 0.9974 | 0.9798 |
| Glycine | 1.0032 | 1.0026 | 1.0109 | 1.0092 | 1.007 | 1.0035 |
| Linolenic acid | 0.9553 | 1.0077 | 1.0186 | 0.9935 | 1.0522 | 1.0602 |
| (C18:cis[9,12,15]3) | | | | | | |
| Behenic acid (C22:0) | 0.9834 | 1.0161 | 0.9871 | 0.9907 | 1.0109 | 1.0087 |
| Dodecanoylcarnitine | 0.9447 | 0.9777 | 0.9598 | 0.9825 | 0.9442 | 0.9456 |
| Phosphatidylcholine | 1.0018 | 1.0018 | 0.9973 | 0.9973 | 0.9959 | 1.0073 |
| (C18:0,C18:2) | | | | | | |
| Stearic acid (C18:0) | 0.9813 | 0.9999 | 0.9936 | 0.9882 | 0.9968 | 1.0061 |
| Palmitic acid (C16:0) | 0.9714 | 1.0048 | 0.9732 | 0.9641 | 1.0052 | 1.0185 |
| Sorbitol | 1.0224 | 1.1391 | 1.0397 | 1.0247 | 1.0005 | 1.0379 |
| Ceramide (d18:1,C24:1) | 0.91 | 0.9512 | 0.9457 | 0.9477 | 0.963 | 0.8865 |
| Hippuric acid | 1.0585 | 1.2351 | 1.1853 | 1.1688 | 1.1782 | 1.2369 |
| Cholesterol, total | 1.0677 | 1.1158 | 1.0991 | 1.1109 | 1.0466 | 1.0621 |
| Arabinose | 1.0802 | 1.1746 | 1.0179 | 1.0725 | 1.0384 | 1.0781 |
| Cortisol | 1.0051 | 0.9617 | 0.9573 | 1.0216 | 1.006 | 0.933 |
| Lauric acid (C12:0) | 0.901 | 1.1288 | 0.9796 | 1.0678 | 0.9208 | 1.1324 |
| Arachidonic acid | 0.9909 | 1.016 | 1.0017 | 1.0018 | 0.9987 | 1.0113 |
| (C20:cis[5,8,11,14]4) | | | | | | |
| 5-Oxoproline | 1.0157 | 1.0388 | 1.0094 | 1.0273 | 1.0277 | 1.0215 |
| Eicosapentaenoic acid | 1.0009 | 1.0342 | 0.9983 | 0.9957 | 0.9824 | 1.0006 |
| (C20:cis[5,8,11,14,17]5) | | | | | | |
| Uridine | 0.9562 | 1.0801 | 1.0467 | 0.9117 | 0.94 | 1.0393 |
| Tricosanoic acid (C23:0) | 0.9893 | 0.9879 | 0.9452 | 0.9515 | 1.0603 | 1.0226 |
| 4-Hydroxy-3- | 1.0182 | 0.98 | 0.9777 | 0.9613 | 1.0111 | 1.0286 |
| methoxyphenylglycol | | | | | | |
| (HMPG) | | | | | | |
| Tetradecanoylcarnitine | 1.0184 | 1.0107 | 0.9755 | 0.9993 | 1.0354 | 0.9959 |
| O-Phosphoethanolamine | 1.0244 | 0.9554 | 1.0125 | 0.7756 | 1.1453 | 1.0012 |
| Erucic acid (C22:cis[13]1) | 0.9886 | 1.2317 | 0.9357 | 1.1287 | 0.9735 | 1.1257 |
| Pantothenic acid | 1.023 | 0.9871 | 0.8618 | 1.007 | 1.0378 | 1.041 |
| Normetanephrine | 1.0393 | 1.0574 | 1.0705 | 1.0668 | 1.0812 | 1.14 |
| Palmitoleic acid | 0.9671 | 1.0031 | 1.0144 | 0.9697 | 1.0333 | 1.0525 |
| (C16:cis[9]1) | | | | | | |
| Fumarate | 1.0135 | 0.9825 | 1.0684 | 0.984 | 1.0373 | 1.0495 |
| Cholesterol, free | 1.0054 | 1.0066 | 0.9988 | 1.0037 | 0.9699 | 0.9927 |
| Cholesta-2,4-dien | 0.9427 | 0.8992 | 0.9555 | 0.9726 | 0.9891 | 0.9454 |
| Creatinine | 1.0431 | 1.0619 | 1.0639 | 1.0828 | 1.0239 | 0.9808 |
| beta-Carotene | 1.0017 | 1.1264 | 1.0148 | 1.0933 | 1.1507 | 1.0812 |
| erythro-Dihydrosphingosine | 0.9796 | 1.0101 | 0.9927 | 1.0091 | 1.0371 | 1.0226 |
| (d18:0) | | | | | | |
| 11.12- | 0.9516 | 0.9232 | 0.9513 | 0.9293 | 0.8928 | 0.9938 |
| Dihydroxyeicosatrienoic | | | | | | |
| acid (C20:cis[5,8,14]3) | | | | | | |
| Ketoleucine | 0.9805 | 1.031 | 1.0373 | 1.0595 | 1.0372 | 1.0311 |

| | | |
|---|---|---|
| Biomarker (Metabolite) | p-value | p-value |

lower part:

| | | | | | | |
|---|---|---|---|---|---|---|
| 3,4- | 0.3231 | 0.2862 | 0.0020 | 1.99E−20 | 0.6473 | 0.7054 |
| Dihydroxyphenylacetic | | | | | | |
| acid | | | | | | |
| (DOPAC) | | | | | | |
| 5- | 0.8036 | 0.0338 | 0.0003 | 1.32E−27 | 0.8000 | 0.0024 |
| Hydroxyeicosatetraenoic | | | | | | |
| acid | | | | | | |
| (C20:trans[6]cis | | | | | | |
| [8,11,14]4) (5- | | | | | | |
| HETE) | | | | | | |
| 12- | 0.2474 | 0.0237 | 0.000009 | 4.89E−22 | 0.0286 | 0.00000002 |
| Hydroxyeicosatetraenoic | | | | | | |
| acid | | | | | | |
| (C20:cis[5,8,10,14]4) | | | | | | |
| Glutamate | 0.3859 | 0.0019 | 0.000001 | 0.00128 | 0.5957 | 0.0116 |
| 15- | 0.3959 | 0.2161 | 0.0030 | 1.1E−11 | 0.1452 | 0.0319 |
| Hydroxyeicosatetraenoic | | | | | | |
| acid | | | | | | |
| (C20:cis[5,8,11,13]4) | | | | | | |
| 3,4- | 0.6080 | 0.2878 | 0.4461 | 0.0001 | 0.5901 | 0.6174 |
| Dihydroxyphenylglycol | | | | | | |
| (DOPEG) | | | | | | |
| 11- | 0.3353 | 0.1704 | 0.00004 | 4.72E−14 | 0.4380 | 0.0015 |
| Hydroxyeicosatetraenoic | | | | | | |
| acid | | | | | | |
| (C20:cis[5,8,12,14]4) | | | | | | |
| 3,4- | 0.8133 | 0.3874 | 0.7948 | 0.0071 | 0.8537 | 0.6380 |
| Dihydroxyphenylalanine | | | | | | |
| (DOPA) | | | | | | |
| 8- | 0.7136 | 0.2540 | 0.2351 | 0.0018 | 0.7629 | 0.1054 |
| Hydroxyeicosatetraenoic | | | | | | |
| acid | | | | | | |
| (C20:trans[5]cis[9,11,14]4) | | | | | | |
| (8- | | | | | | |
| HETE) | | | | | | |
| Prostaglandin D2 | 0.4480 | 0.0372 | 0.0335 | 0.0068 | 0.9000 | 0.0025 |
| Maltose | 0.8409 | 0.0007 | 0.0008 | 2.86E−18 | 0.0054 | 0.000004 |
| alpha- | 0.6950 | 0.2121 | 0.4686 | 0.3395 | 0.0513 | 0.4883 |
| Ketoglutarate | | | | | | |
| Noradrenaline | 0.5246 | 0.1967 | 0.0265 | 0.0016 | 0.7048 | 0.1456 |
| (Norepinephrine) | | | | | | |
| Cysteine | 0.9703 | 0.0330 | 0.0001 | 1.26E−11 | 0.6599 | 0.0924 |
| Glutamate to | 0.8068 | 0.0234 | 0.0019 | 0.3898 | 0.0739 | 0.0019 |
| glutamine intra- | | | | | | |
| sample ratio | | | | | | |
| Glycerate | 0.2871 | 0.1532 | 0.0001 | 1.07E−13 | 0.8723 | 0.0522 |
| 8,9- | 0.7831 | 0.1138 | 0.2446 | 0.0001 | 0.1694 | 0.3962 |
| Dihydroxyeicosatrienoic | | | | | | |
| acid | | | | | | |
| (C20:cis[5,11,14]3) | | | | | | |
| Threonic acid | 0.0033 | 1.04E−08 | 1.15E−14 | 2.76E−19 | 0.000002 | 3.85E−17 |
| delta-12- | 0.5210 | 0.1644 | 0.2916 | 0.000004 | 0.8371 | 0.0434 |
| Prostaglandin D2 | | | | | | |
| Prostaglandin E2 | 0.0018 | 3.37E−08 | 5.14E−11 | 7.52E−17 | 0.0695 | 4.64E−09 |
| Glycerol-3- | 0.1404 | 0.1619 | 0.3295 | 0.0204 | 0.7054 | 0.2153 |
| phosphate, polar | | | | | | |
| fraction | | | | | | |
| Lysophosphatidylcholine | 0.0175 | 0.05560 | 0.0394 | 0.0015 | 0.5639 | 0.8132 |
| (C17:0) | | | | | | |
| Pyruvate | 0.7561 | 0.9532 | 0.1034 | 0.0406 | 0.4925 | 0.6482 |
| 12- | 0.4880 | 0.4223 | 0.5486 | 0.1802 | 0.0840 | 0.2996 |
| Hydroxyheptadecatrienoic | | | | | | |
| acid | | | | | | |
| (C17:[5,8,10]3) | | | | | | |
| 13- | 0.2236 | 0.70581 | 0.3718 | 0.9013 | 0.4875 | 0.3245 |
| Hydroxyoctadecadienoic | | | | | | |
| acid | | | | | | |
| (13-NODE) | | | | | | |
| (C18:cis[9]trans[11]2) | | | | | | |
| Glutamine | 0.8828 | 0.59588 | 0.4580 | 0.8632 | 0.4577 | 0.1995 |
| Adrenaline (Epinephrine) | 0.6575 | 0.4306 | 0.6751 | 0.0645 | 0.8934 | 0.9533 |
| 3- | 0.8569 | 0.6808 | 0.0904 | 0.0002 | 0.1045 | 0.7445 |
| Phosphoglycerate | | | | | | |
| (3-PGA) | | | | | | |
| Lysophosphatidylcholine | 0.3162 | 0.5632 | 0.6429 | 0.5422 | 0.5383 | 0.6886 |
| (C18:0) | | | | | | |
| Thromboxane B2 | 0.7617 | 0.5271 | 0.4663 | 0.4171 | 0.0187 | 0.0652 |
| 9- | 0.5788 | 0.9048 | 0.6041 | 0.9337 | 0.2474 | 0.5030 |
| Hydroxyoctadecadienoic | | | | | | |
| acid | | | | | | |
| (9-HODE) | | | | | | |
| (C18:trans[10]cis | | | | | | |
| [12]2) | | | | | | |
| Cystine | 0.9558 | 0.0486 | 0.4356 | 0.0004 | 0.6962 | 0.3247 |
| Phosphatidylcholine | 0.8954 | 0.3488 | 0.4212 | 0.1180 | 0.7331 | 0.5648 |
| (C16:1,C18:2) | | | | | | |
| Alanine | 0.9717 | 0.8702 | 0.5322 | 0.9019 | 0.7732 | 0.6620 |
| Glycerol, polar | 0.3619 | 0.7595 | 0.4993 | 0.8567 | 0.6481 | 0.5676 |
| fraction | | | | | | |
| Isocitrate | 0.3997 | 0.0376 | 0.0322 | 0.0001 | 0.6392 | 0.2873 |
| Lysophosphatidylcholine | 0.4550 | 0.5191 | 0.7855 | 0.0197 | 0.4129 | 0.0210 |
| (C20:4) | | | | | | |
| 1-Hydroxy-2- | 0.0798 | 0.0209 | 0.1261 | 0.2301 | 0.0638 | 0.1689 |
| amino-(cis.trans)- | | | | | | |
| 3,5- | | | | | | |
| octadecadiene | | | | | | |
| (from sphingolipids) | | | | | | |
| Lysophosphatidylcholine | 0.4466 | 0.8903 | 0.9681 | 0.5975 | 0.5366 | 0.6817 |
| (C18:1) | | | | | | |
| Ceramide | 0.3508 | 0.1951 | 0.3760 | 0.0280 | 0.0494 | 0.6599 |
| (d18:1,C24:0) | | | | | | |
| 14,15- | 0.3680 | 0.2948 | 0.4768 | 0.1166 | 0.5683 | 0.5527 |
| Dihydroxyeicosatrienoic | | | | | | |
| acid | | | | | | |
| (C20:cis[5,8,11]3) | | | | | | |
| Lysophosphatidylcholine | 0.0070 | 0.1868 | 0.2042 | 0.9260 | 0.4695 | 0.1324 |
| (C18:2) | | | | | | |
| erythro- | 0.0102 | 0.0024 | 0.0063 | 0.0114 | 0.0582 | 0.0887 |
| Dihydrosphingosine | | | | | | |
| (d16:0) | | | | | | |
| Valine | 0.6468 | 0.4919 | 0.4161 | 0.5405 | 0.5228 | 0.6556 |
| erythro- | 0.0288 | 0.0027 | 0.0117 | 0.0191 | 0.1096 | 0.1434 |
| Sphingosine | | | | | | |
| (d18:1) | | | | | | |
| Creatine | 0.1998 | 0.0166 | 0.2575 | 0.3443 | 0.6235 | 0.6220 |
| myo-Inositol-2- | 0.6985 | 0.1686 | 0.5345 | 0.3048 | 0.0838 | 0.1450 |
| phosphate, lipid | | | | | | |
| fraction (myo- | | | | | | |
| Inositolphospho- | | | | | | |
| lipids) | | | | | | |
| Leucine | 0.6333 | 0.9934 | 0.9008 | 0.7800 | 0.9355 | 0.7898 |
| Quinic acid | 0.8034 | 0.1471 | 0.4306 | 0.7870 | 0.1329 | 0.4214 |
| Glycerol, lipid | 0.1025 | 0.8896 | 0.2090 | 0.2218 | 0.4109 | 0.5506 |
| fraction | | | | | | |
| Lysophosphatidylcholine | 0.9902 | 0.4495 | 0.1206 | 0.0070 | 0.9048 | 0.2496 |
| (C16:0) | | | | | | |
| Eicosanoic acid | 0.1462 | 0.8994 | 0.1956 | 0.6040 | 0.0084 | 0.0822 |
| (C20:0) | | | | | | |
| Octadecanoylcarnitine | 0.1383 | 0.6487 | 0.1011 | 0.2908 | 0.5737 | 0.0318 |
| Phosphatidylcholine | 0.6077 | 0.0916 | 0.5842 | 0.1609 | 0.7089 | 0.4158 |
| (C18:0,C18:1) | | | | | | |
| Serine | 0.5824 | 0.0096 | 0.4824 | 0.1157 | 0.1507 | 0.6571 |
| Erythrol | 0.7155 | 0.0100 | 0.2285 | 0.8183 | 0.9348 | 0.7438 |
| Phosphatidylcholine | 0.0582 | 0.1534 | 0.4574 | 0.8730 | 0.8464 | 0.4436 |
| (C16:0,C16:0) | | | | | | |
| Glucose-6- | 0.4918 | 0.9450 | 0.2298 | 0.1607 | 0.5562 | 0.6566 |
| phosphate | | | | | | |
| Cholesta-2,4,6- | 0.1251 | 0.3738 | 0.1876 | 0.2710 | 0.0811 | 0.0467 |
| triene | | | | | | |
| trans-4- | 0.9978 | 0.8960 | 0.1492 | 0.8519 | 0.5570 | 0.3739 |
| Hydroxyproline | | | | | | |
| Cholesterylester | 0.2254 | 0.7091 | 0.4723 | 0.7983 | 0.8675 | 0.7227 |
| C18:2 | | | | | | |
| Docosahexaenoic | 0.5865 | 0.7553 | 0.8501 | 0.6182 | 0.6042 | 0.4485 |
| acid | | | | | | |
| (C22:cis[4,7,10,13, | | | | | | |
| 16,19]6) | | | | | | |
| 4- | 0.8129 | 0.9399 | 0.8092 | 0.8489 | 0.1006 | 0.0442 |
| Hydroxysphinganine | | | | | | |
| (t18:0, Phytosphingosine), | | | | | | |
| total | | | | | | |
| 14- | 0.3645 | 0.6920 | 0.2476 | 0.5005 | 0.6048 | 0.7960 |
| Methylhexadecanoic | | | | | | |
| acid | | | | | | |
| TAG(C16:0,C18: | 0.1868 | 0.3793 | 0.7711 | 0.3563 | 0.9298 | 0.4928 |
| 1,C18:2) | | | | | | |
| Glycine | 0.5825 | 0.6529 | 0.0606 | 0.1687 | 0.2256 | 0.5481 |
| Linolenic acid | 0.3213 | 0.8680 | 0.6906 | 0.9007 | 0.2773 | 0.2103 |
| (C18:cis[9,12,15] | | | | | | |
| 3) | | | | | | |
| Behenic acid | 0.4259 | 0.4481 | 0.5380 | 0.6959 | 0.6136 | 0.6842 |
| (C22:0) | | | | | | |
| Dodecanoylcarnitine | 0.1332 | 0.5558 | 0.2887 | 0.6824 | 0.1222 | 0.1351 |
| Phosphatidylcholine | 0.8288 | 0.8350 | 0.7513 | 0.7758 | 0.6183 | 0.3745 |
| (C18:0,C18:2) | | | | | | |
| Stearic acid | 0.1702 | 0.9955 | 0.6429 | 0.4484 | 0.818 | 0.6598 |
| (C18:0) | | | | | | |
| Palmitic acid | 0.1037 | 0.7903 | 0.1286 | 0.0728 | 0.7734 | 0.3101 |
| (C16:0) | | | | | | |
| Sorbitol | 0.6923 | 0.0223 | 0.4885 | 0.7037 | 0.992 | 0.5078 |
| Ceramide | 0.0783 | 0.3545 | 0.3074 | 0.3783 | 0.4721 | 0.0232 |
| (d18:1,C24:1) | | | | | | |
| Hippuric acid | 0.6009 | 0.0545 | 0.1206 | 0.2110 | 0.1312 | 0.0512 |
| Cholesterol, total | 0.1801 | 0.0258 | 0.0545 | 0.0596 | 0.3586 | 0.2229 |
| Arabinose | 0.2857 | 0.0264 | 0.8041 | 0.3943 | 0.5979 | 0.2926 |
| Cortisol | 0.8921 | 0.3035 | 0.2558 | 0.6165 | 0.8712 | 0.0622 |
| Lauric acid | 0.4242 | 0.3541 | 0.8746 | 0.6590 | 0.5336 | 0.3463 |
| (C12:0) | | | | | | |
| Arachidonic acid | 0.5252 | 0.2699 | 0.9057 | 0.9130 | 0.9275 | 0.4382 |
| (C20:cis[5,8,11,14] | | | | | | |
| 4) | | | | | | |
| 5-Oxoproline | 0.6499 | 0.2672 | 0.7839 | 0.4901 | 0.4208 | 0.5294 |
| Eicosapentaenoic | 0.9837 | 0.4206 | 0.9669 | 0.9279 | 0.6748 | 0.9895 |
| acid | | | | | | |
| (C20:cis[5,8,11,14, | | | | | | |
| 17]5) | | | | | | |
| Uridine | 0.3698 | 0.1261 | 0.3635 | 0.1070 | 0.2148 | 0.4406 |
| Tricosanoic acid | 0.7678 | 0.7384 | 0.1231 | 0.2306 | 0.1137 | 0.5438 |
| (C23:0) | | | | | | |
| 4-Hydroxy-3- | 0.5891 | 0.5489 | 0.5009 | 0.3036 | 0.7390 | 0.3962 |
| methoxyphenylglycol | | | | | | |
| (HMPG) | | | | | | |
| Tetradecanoylcarnitine | 0.6303 | 0.7813 | 0.5228 | 0.9879 | 0.3507 | 0.9117 |
| O- | 0.8208 | 0.6706 | 0.9078 | 0.0382 | 0.2021 | 0.9908 |
| Phosphoethanolamine | | | | | | |
| Erucic acid | 0.9195 | 0.0669 | 0.5578 | 0.3483 | 0.8155 | 0.3012 |
| (C22:cis[13]1) | | | | | | |
| Pantothenic acid | 0.7493 | 0.8556 | 0.0383 | 0.9315 | 0.6005 | 0.5722 |
| Normetanephrine | 0.5472 | 0.3859 | 0.2889 | 0.3784 | 0.2195 | 0.0399 |
| Palmitoleic acid | 0.1843 | 0.9031 | 0.5704 | 0.2842 | 0.2010 | 0.0453 |
| (C16:cis[9]1) | | | | | | |
| Fumarate | 0.7869 | 0.7222 | 0.1813 | 0.7761 | 0.4568 | 0.3263 |
| Cholesterol, free | 0.8476 | 0.8160 | 0.9655 | 0.9069 | 0.2675 | 0.7915 |
| Cholesta-2,4- | 0.3281 | 0.0797 | 0.4520 | 0.6859 | 0.8578 | 0.3577 |
| dien | | | | | | |
| Creatinine | 0.4339 | 0.2681 | 0.2528 | 0.1981 | 0.6597 | 0.7192 |
| beta-Carotene | 0.9806 | 0.0928 | 0.8373 | 0.2631 | 0.0412 | 0.2579 |
| erythro- | 0.4105 | 0.6894 | 0.7706 | 0.7504 | 0.1535 | 0.3770 |
| Dihydrosphingosine | | | | | | |
| (d18:0) | | | | | | |
| 11,12- | 0.3734 | 0.1526 | 0.3708 | 0.2514 | 0.0415 | 0.9100 |
| Dihydro- | | | | | | |
| xyeicosatrienoic | | | | | | |
| acid | | | | | | |
| (C20:cis[5,8,14]3) | | | | | | |
| Ketoleucine | 0.6064 | 0.4252 | 0.3356 | 0.1851 | 0.3350 | 0.4185 |

| | | | | | | |
|---|---|---|---|---|---|---|
| Temp. ° C. | 12 | 12 | 21 | 21 | 21 | 21 |
| Time | 5 | 16 | 0.5 | 2 | 5 | 16 |

| | | |
|---|---|---|
| | Ratio relative | |
| Biomarker (Metabolite) | to = 0 at 12° C. | Ratio relative to = 0 at 21° c. |

upper part:

| | | | | | | |
|---|---|---|---|---|---|---|
| 3,4-Dihydroxyphenylacetic | 0.7632 | 0.2692 | 0.9514 | 0.7159 | 0.4429 | 0.0426 |
| acid (DOPAC) | | | | | | |
| 5-Hydroxyeicosatetraenoic | 1.3103 | 2.8347 | 1.0687 | 1.3847 | 2.1244 | 7.8028 |
| acid | | | | | | |
| (C20:trans[6]cis[8,11,14]4) | | | | | | |
| (5-HETE) | | | | | | |
| 12- | 1.8759 | 4.1526 | 1.1067 | 2.9096 | 5.3703 | 10.4655 |
| Hydroxyeicosatetraenoic | | | | | | |
| acid (C20:cis[5,8,10,14]4) | | | | | | |
| Glutamate | 1.302 | 1.7501 | 1.0439 | 1.3227 | 1.8253 | 5.0316 |
| 15- | 1.1412 | 1.6792 | 0.9664 | 1.3509 | 1.719 | 2.8933 |
| Hydroxyeicosatetraenoic | | | | | | |
| acid (C20:cis[5,8,11,13]4) | | | | | | |
| 3,4-Dihydroxyphenylglycol | 1.0236 | 0.6381 | 1.0227 | 0.9964 | 0.8251 | 0.2795 |
| (DOPEG) | | | | | | |
| 11- | 1.2861 | 2.024 | 1.0208 | 1.3727 | 1.6757 | 2.9929 |
| Hydroxyeicosatetraenoic | | | | | | |
| acid (C20:cis[5,8,12,14]4) | | | | | | |
| 3,4- | 0.965 | 0.7188 | 1.0023 | 0.9234 | 0.7673 | 0.291 |
| Dihydroxyphenylalanine | | | | | | |
| (DOPA) | | | | | | |
| 8-Hydroxyeicosatetraenoic | 1.1601 | 1.5694 | 1.0447 | 1.2211 | 1.5183 | 2.7721 |
| acid | | | | | | |
| (C20:trans[5]cis[9,11,14]4) | | | | | | |
| (8-HETE) | | | | | | |
| Prostaglandin D2 | 1.0043 | 2.5233 | 1.0761 | 1.1664 | 1.7616 | 13.715 |
| Maltose | 1.4124 | 2.1686 | 1.101 | 1.4244 | 1.8475 | 2.6055 |
| alpha-Ketoglutarate | 1.1565 | 1.5065 | 1.0284 | 1.1004 | 1.2106 | 3.343 |
| Noradrenaline | 1.002 | 0.8115 | 1.0052 | 0.9519 | 0.8401 | 0.3506 |
| (Norepinephrine) | | | | | | |
| Cysteine | 0.8818 | 0.7549 | 0.9661 | 0.8353 | 0.765 | 0.6082 |
| Glutamate to glutamine | 1.713 | 2.2991 | 1.0241 | 1.289 | 1.9656 | 7.5462 |
| intra-sample ratio | | | | | | |
| Glycerate | 1.2341 | 2.0877 | 0.9817 | 1.1929 | 1.6299 | 2.3937 |
| 8,9- | 1.1318 | 1.4552 | 1.0415 | 1.1298 | 1.34 | 1.992 |
| Dihydroxyeicosatrienoic | | | | | | |
| acid (C20:cis[5,11,14]3) | | | | | | |
| Threonic acid | 1.9498 | 2.0835 | 1.2587 | 1.7045 | 1.8148 | 2.134 |
| delta-12-Prostaglandin D2 | 1.6812 | 11.7384 | 2.2502 | 3.0662 | 5.2773 | 60.7641 |
| Prostaglandin E2 | 1.6404 | 2.8443 | 1.465 | 1.6039 | 2.2792 | 3.7779 |
| Glycerol-3-phosphate, polar | 1.0258 | 1.3689 | 1.0469 | 1.0583 | 1.3728 | 2.83 |
| fraction | | | | | | |
| Lysophosphatidylcholine | 1.0564 | 1.1236 | 1.0458 | 1.1217 | 1.1965 | 1.8146 |
| (C17:0) | | | | | | |
| Pyruvate | 1.0093 | 0.9512 | 0.9907 | 0.9937 | 0.9427 | 0.7931 |
| 12- | 1.0228 | 1.0918 | 1.1087 | 1.6634 | 1.6591 | 1.8024 |
| Hydroxyheptadecatrienoic | | | | | | |
| acid (C17:[5,8,10]3) | | | | | | |
| 13- | 1.0278 | 1.1809 | 1.006 | 1.0817 | 1.1575 | 1.3839 |
| Hydroxyoctadecadienoic | | | | | | |
| acid (13-HODE) | | | | | | |
| (C18:cis[9]trans[11]2) | | | | | | |
| Glutamine | 0.8382 | 0.9021 | 0.9866 | 0.9915 | 0.9474 | 0.6024 |
| Adrenaline (Epinephrine) | 1.0947 | 0.85 | 1.0549 | 0.9936 | 0.88 | 0.4667 |
| 3-Phosphoglycerate (3- | 1.2527 | 1.6956 | 1.1811 | 1.3687 | 1.6791 | 2.4618 |
| PGA) | | | | | | |
| Lysophosphatidylcholine | 1.0749 | 1.0909 | 1.0573 | 1.075 | 1.0986 | 1.4834 |
| (C18:0) | | | | | | |
| Thromboxane B2 | 0.868 | 0.9066 | 1.1174 | 1.5133 | 1.6579 | 1.6397 |
| 9-Hydroxyoctadecadienoic | 1.0142 | 1.1401 | 0.994 | 1.0409 | 1.0944 | 1.235 |
| acid (9-HODE) | | | | | | |
| (C18:trans[10]cis[12]2) | | | | | | |
| Cystine | 0.8784 | 0.8098 | 0.9478 | 0.8922 | 0.812 | 0.6584 |
| Phosphatidylcholine | 0.9933 | 0.9949 | 1.0097 | 1.0076 | 0.9812 | 0.9164 |
| (C16:1,C18:2) | | | | | | |
| Alanine | 1.0089 | 1.0071 | 0.9822 | 0.9859 | 0.9934 | 1.0402 |
| Glycerol, polar fraction | 1.0578 | 1.0604 | 0.9542 | 0.9984 | 1.0044 | 1.1567 |
| Isocitrate | 1.0532 | 1.0764 | 1.0355 | 1.0394 | 1.0734 | 1.1648 |
| Lysophosphatidylcholine | 0.9767 | 1.0397 | 0.9771 | 1.0142 | 0.9682 | 1.1605 |
| (C20:4) | | | | | | |
| 1-Hydroxy-2-amino- | 1.0572 | 1.018 | 1.1182 | 1.0971 | 1.0538 | 1.0876 |
| (cis.trans)-3,5- | | | | | | |
| octadecadiene (from | | | | | | |
| sphingolipids) | | | | | | |
| Lysophosphatidylcholine | 0.9893 | 1.03 | 1.0127 | 1.0519 | 1.0599 | 1.143 |
| (C18:1) | | | | | | |
| Ceramide (d18:1,C24:0) | 1.036 | 0.9962 | 1.0179 | 1.0334 | 0.9796 | 1.1926 |
| 14.15- | 1.0354 | 0.9988 | 1.0363 | 1.0313 | 1.083 | 1.2433 |
| Dihydroxyeicosatrienoic | | | | | | |
| acid (C20:cis[5,8,11]3) | | | | | | |
| Lysophosphatidylcholine | 0.8946 | 0.8867 | 0.8606 | 0.8866 | 0.8338 | 0.8128 |
| (C18:2) | | | | | | |
| erythro-Dihydrosphingosine | 1.1028 | 1.0588 | 1.1244 | 1.1125 | 1.0694 | 1.1139 |
| (d16:0) | | | | | | |
| Valine | 1.0037 | 1.0035 | 0.9832 | 0.989 | 0.9717 | 0.9965 |
| erythro-Sphingosine | 1.0765 | 1.0566 | 1.1059 | 1.1134 | 1.0646 | 1.0998 |
| (d18:1) | | | | | | |
| Creatine | 1.0479 | 1.0255 | 1.1138 | 1.0948 | 1.1083 | 1.0438 |
| myo-Inositol-2-phosphate. | 1.1756 | 1.0276 | 1.3109 | 1.1683 | 1.3621 | 1.1927 |
| lipid fraction (myo- | | | | | | |
| Inositolphospholipids) | | | | | | |
| Leucine | 1.0022 | 0.9983 | 0.9863 | 0.9905 | 0.9755 | 0.9933 |
| Quinic acid | 1.0651 | 1.0809 | 1.0122 | 1.0376 | 1.0198 | 1.1121 |
| Glycerol, lipid fraction | 1.017 | 1.0372 | 0.9621 | 1.0073 | 0.9232 | 0.9611 |
| Lysophosphatidylcholine | 0.9736 | 1.0067 | 1.03 | 0.9585 | 1.0356 | 1.1657 |
| (C16:0) | | | | | | |
| Eicosanoic acid (C20:0) | 0.9786 | 0.9482 | 0.9761 | 0.9672 | 0.9567 | 0.9586 |
| Octadecanoylcarnitine | 0.9604 | 0.9676 | 1.028 | 0.9805 | 0.9328 | 0.9608 |
| Phosphatidylcholine | 0.9927 | 0.9879 | 0.9951 | 1.0001 | 0.9874 | 0.9758 |
| (C18:0.C18:1) | | | | | | |
| Serine | 1.0104 | 1.0206 | 1.0039 | 1.0052 | 0.9878 | 1.011 |
| Erythrol | 0.954 | 1.0017 | 1.0236 | 1.017 | 0.9414 | 0.9768 |
| Phosphatidylcholine | 0.985 | 0.9921 | 0.97 | 0.9872 | 0.9556 | 0.9571 |
| (C16:0.C16:0) | | | | | | |
| Glucose-6-phosphate | 1.0566 | 1.1087 | 0.9186 | 1.1209 | 1.1887 | 1.5598 |
| Cholesta-2,4,6-triene | 0.9268 | 0.8506 | 0.8901 | 0.8516 | 0.9697 | 0.8075 |
| trans-4-Hydroxyproline | 0.9956 | 1.0515 | 0.9936 | 0.9974 | 1.0052 | 1.003 |
| Cholesterylester C18:2 | 1.0212 | 0.9945 | 1.0215 | 1.0399 | 1.0457 | 1.0769 |
| Docosahexaenoic acid | 1.0325 | 1.0275 | 1.0227 | 1.0428 | 1.0215 | 1.0374 |
| (C22:cis[4,7,10,13,16,19]6) | | | | | | |
| 4-Hydroxysphinganine | 1.1193 | 0.9587 | 1.064 | 1.0777 | 1.0145 | 1.013 |
| (t18:0, Phytosphingosine), | | | | | | |
| total | | | | | | |
| 14-Methylhexadecanoic | 1.021 | 1.0755 | 1.0306 | 1.0264 | 1.0006 | 1.0346 |
| acid | | | | | | |
| TAG(C16:0,C18:1,C18:2) | 0.9858 | 0.9338 | 0.9816 | 0.9775 | 0.9493 | 0.9226 |
| Glycine | 1.0074 | 1.0159 | 1.0009 | 1.003 | 1.0038 | 1.0028 |
| Linolenic acid | 1.0641 | 1.14 | 0.9402 | 1.0365 | 1.033 | 1.0307 |
| (C18:cis[9,12,15]3) | | | | | | |
| Behenic acid (C22:0) | 1.0267 | 1.0133 | 1.0228 | 1.0498 | 1.0132 | 1.0035 |
| Dodecanoylcarnitine | 0.9153 | 0.9208 | 0.9816 | 0.9767 | 0.9657 | 0.9864 |
| Phosphatidylcholine | 1.0194 | 1.0083 | 0.9941 | 0.9958 | 0.9955 | 1.0176 |
| (C18:0,C18:2) | | | | | | |
| Stearic acid (C18:0) | 1.0203 | 1.0387 | 0.995 | 1.0245 | 0.986 | 0.9994 |
| Palmitic acid (C16:0) | 1.0068 | 1.0256 | 0.9863 | 1.0185 | 0.9604 | 0.9968 |
| Sorbitol | 1.0856 | 1.0036 | 1.0027 | 0.9999 | 0.9887 | 1.022 |
| Ceramide (d18:1,C24:1) | 0.9206 | 1.0001 | 0.9933 | 1.0147 | 0.9859 | 1.1437 |
| Hippuric acid | 1.2832 | 1.1631 | 1.0285 | 0.9269 | 0.9097 | 0.9722 |
| Cholesterol, total | 1.062 | 1.0495 | 1.075 | 1.1135 | 1.0509 | 1.1079 |
| Arabinose | 1.0496 | 0.9723 | 0.9997 | 0.9574 | 0.9023 | 0.9452 |
| Cortisol | 0.9627 | 0.9496 | 0.9918 | 0.9652 | 0.9228 | 0.9406 |
| Lauric acid (C12:0) | 1.0153 | 1.4139 | 0.9962 | 1.1811 | 0.9005 | 1.0423 |
| Arachidonic acid | 1.0207 | 1.0389 | 1.0031 | 1.0315 | 0.9914 | 0.9999 |
| (C20:cis[5,8,11,14]4) | | | | | | |
| 5-Oxoproline | 1.0766 | 1.082 | 0.9866 | 0.9676 | 1.0037 | 1.0493 |
| Eicosapentaenoic acid | 0.9539 | 0.9389 | 0.9145 | 0.9905 | 0.9187 | 0.9417 |
| (C20:cis[5,8,11,14,17]5) | | | | | | |
| Uridine | 0.9614 | 1.0271 | 1.045 | 1.1105 | 1.0782 | 1.048 |
| Tricosanoic acid (C23:0) | 1.0359 | 1.0257 | 1.0413 | 1.0791 | 1.0129 | 1.0015 |
| 4-Hydroxy-3- | 0.9961 | 0.9252 | 0.9978 | 0.9769 | 0.9329 | 0.9438 |
| methoxyphenylglycol | | | | | | |
| (HMPG) | | | | | | |
| Tetradecanoylcarnitine | 0.959 | 0.9985 | 1.0791 | 0.9864 | 0.9702 | 0.9882 |
| O-Phosphoethanolamine | 1.1156 | 0.9336 | 0.9646 | 0.9282 | 1.1203 | 0.9773 |
| Erucic acid (C22:cis[13]1) | 0.9978 | 0.8543 | 0.9331 | 1.2604 | 1.2163 | 1.1196 |
| Pantothenic acid | 0.9416 | 0.9929 | 1.0425 | 1.0341 | 0.9065 | 0.9706 |
| Normetanephrine | 1.1442 | 1.0427 | 0.9753 | 1.047 | 1.1174 | 1.0903 |
| Palmitoleic acid | 1.0553 | 1.0451 | 0.9726 | 1.0041 | 0.9639 | 0.9767 |
| (C16:cis[9]1) | | | | | | |
| Fumarate | 1.0817 | 1.0393 | 1.0303 | 1.0411 | 1.1052 | 1.0538 |
| Cholesterol, free | 0.9845 | 0.959 | 1.0102 | 1.0233 | 0.98 | 0.9374 |
| Cholesta-2,4-dien | 1.0302 | 0.8607 | 0.912 | 0.906 | 0.9278 | 0.9496 |
| Creatinine | 1.0659 | 0.9967 | 1.1147 | 1.0442 | 1.0621 | 0.965 |
| beta-Carotene | 1.136 | 1.1173 | 1.0939 | 1.1081 | 1.0512 | 1.0803 |
| erythro-Dihydrosphingosine | 1.0527 | 1.0445 | 1.0527 | 1.0438 | 0.9965 | 1.0457 |
| (d18:0) | | | | | | |
| 11.12- | 0.8948 | 1.0325 | 0.9531 | 1.0411 | 0.9891 | 1.0187 |
| Dihydroxyeicosatrienoic | | | | | | |
| acid (C20:cis[5,8,14]3) | | | | | | |
| Ketoleucine | 1.0401 | 1.0925 | 1.0262 | 1.0422 | 1.0258 | 1.0523 |

| | | |
|---|---|---|
| Biomarker (Metabolite) | p-value | p-value |

lower part:

| | | | | | | |
|---|---|---|---|---|---|---|
| 3,4- | 0.000019 | 1.2E−45 | 0.4139 | 9.57E−08 | 1.34E−30 | 1.6E−111 |
| Dihydroxyphenylacetic | | | | | | |
| acid | | | | | | |
| (DOPAC) | | | | | | |
| 5- | 2.4E−09 | 6.29E−55 | 0.1341 | 3.17E−12 | 1.01E−42 | 4.92E−107 |
| Hydroxyeicosatetraenoic | | | | | | |
| acid | | | | | | |
| (C20:trans[6]cis | | | | | | |
| [8,11,14]4) (5- | | | | | | |
| HETE) | | | | | | |
| 12- | 1.14E−20 | 4.67E−53 | 0.1042 | 3.65E−43 | 1.68E−73 | 2.72E−89 |
| Hydroxyeicosatetraenoic | | | | | | |
| acid | | | | | | |
| (C20:cis[5,8,10,14]4) | | | | | | |
| Glutamate | 2.22E−08 | 7.45E−22 | 0.3421 | 2.36E−09 | 1.28E−30 | 4.85E−84 |
| 15- | 0.0001 | 1.62E−31 | 0.3092 | 1.06E−16 | 8.76E−40 | 3.3E−75 |
| Hydroxyeicosatetraenoic | | | | | | |
| acid | | | | | | |
| (C20:cis[5,8,11,13]4) | | | | | | |
| 3,4- | 0.5959 | 5.07E−17 | 0.6014 | 0.9327 | 0.00001 | 2.49E−66 |
| Dihydroxyphenylglycol | | | | | | |
| (DOPEG) | | | | | | |
| 11- | 0.0000003 | 4.16E−29 | 0.6703 | 3.28E−10 | 5.52E−22 | 8.12E−52 |
| Hydroxyeicosatetraenoic | | | | | | |
| acid | | | | | | |
| (C20:cis[5,8,12,14]4) | | | | | | |
| 3,4- | 0.5015 | 8.61E−08 | 0.9644 | 0.1265 | 0.000001 | 1.96E−51 |
| Dihydroxyphenylalanine | | | | | | |
| (DOPA) | | | | | | |
| 8- | 0.0019 | 8.4E−15 | 0.3626 | | | |
| Hydroxyeicosatetraenoic | | | | | | |
| acid | | | | | | |
| (C20:trans[5]cis[9,11,14]4) | | | | | | |
| (8- | | | | | | |
| HETE) | | | | | | |
| Prostaglandin D2 | 0.9741 | 6.19E−09 | 0.5889 | 0.2570 | 0.00004 | 3.73E−42 |
| Maltose | 7.03E−11 | 2.44E−30 | 0.0559 | 1.6E−11 | 3.86E−27 | 2.6E−41 |
| alpha- | 0.0239 | 7.26E−08 | 0.6663 | 0.1311 | 0.0027 | 6.07E−41 |
| Ketoglutarate | | | | | | |
| Noradrenaline | 0.9699 | 0.0007 | 0.9218 | 0.3532 | 0.0012 | 1.3E−40 |
| (Norepinephrine) | | | | | | |
| Cysteine | 0.00001 | 2.37E−16 | 0.2088 | 2.75E−10 | 3.54E−19 | 1.74E−38 |
| Glutamate to | 0.000009 | 4.59E−09 | 0.8396 | 0.0314 | 2.44E−08 | 1.07E−35 |
| glutamine intra- | | | | | | |
| sample ratio | | | | | | |
| Glycerate | 0.0001 | 6.8E−26 | 0.7278 | 0.0010 | 1.47E−17 | 1.2E−33 |
| 8,9- | 0.0039 | 4.45E−13 | 0.3472 | 0.0051 | 9.86E−11 | 1.91E−32 |
| Dihydroxyeicosatrienoic | | | | | | |
| acid | | | | | | |
| (C20:cis[5,11,14]3) | | | | | | |
| Threonic acid | 1.52E−31 | 1.54E−29 | 0.000003 | 4.39E−23 | 2.42E−27 | 1.99E−31 |
| delta-12- | 0.0593 | 1.96E−13 | 0.0039 | 0.0001 | 8.79E−09 | 6.27E−29 |
| Prostaglandin D2 | | | | | | |
| Prostaglandin E2 | 0.0000001 | 4.03E−20 | 0.00004 | 0.000001 | 7.72E−17 | 2.34E−28 |
| Glycerol-3- | 0.7286 | 0.0002 | 0.5304 | 0.4283 | 0.00001 | 3.62E−28 |
| phosphate, polar | | | | | | |
| fraction | | | | | | |
| Lysophosphatidylcholine | 0.2327 | 0.0246 | 0.3114 | 0.0102 | 0.0001 | 5.22E−25 |
| (C17:0) | | | | | | |
| Pyruvate | 0.6273 | 0.0234 | 0.6234 | 0.7366 | 0.001882945 | 7.34E−22 |
| 12- | 0.6558 | 0.1324 | 0.0458 | 1.71E−19 | 2.44E−19 | 8.3E−20 |
| Hydroxyheptadecatrienoic | | | | | | |
| acid | | | | | | |
| (C17:[5,8,10]3) | | | | | | |
| 13- | 0.3278 | 0.0000005 | 0.8335 | 0.0062 | 0.000001 | 1.06E−19 |
| Hydroxyoctadecadienoic | | | | | | |
| acid | | | | | | |
| (13-NODE) | | | | | | |
| (C18:cis[9]trans[11]2) | | | | | | |
| Glutamine | 0.0004 | 0.0682 | 0.7807 | 0.8599 | 0.2626 | 4.31E−17 |
| Adrenaline (Epinephrine) | 0.2310 | 0.0556 | 0.4692 | 0.9312 | 0.0909 | 1.14E−15 |
| 3- | 0.0219 | 0.000002 | 0.1349 | 0.0015 | 0.0000002 | 1.34E−14 |
| Phosphoglycerate | | | | | | |
| (3-PGA) | | | | | | |
| Lysophosphatidylcholine | 0.1186 | 0.0943 | 0.2106 | 0.1062 | 0.0358 | 4.35E−13 |
| (C18:0) | | | | | | |
| Thromboxane B2 | 0.0441 | 0.2243 | 0.1198 | 1.87E−08 | 1.42E−11 | 5.15E−09 |
| 9- | 0.6111 | 0.00005 | 0.8323 | 0.1560 | 0.0016 | 4.01E−10 |
| Hydroxyoctadecadienoic | | | | | | |
| acid | | | | | | |
| (9-HODE) | | | | | | |
| (C18:trans[10]cis | | | | | | |
| [12]2) | | | | | | |
| Cystine | 0.0444 | 0.0047 | 0.4002 | 0.0732 | 0.0011 | 3.22E−08 |
| Phosphatidylcholine | 0.6543 | 0.7608 | 0.5048 | 0.6048 | 0.1935 | 0.0000004 |
| (C16:1,C18:2) | | | | | | |
| Alanine | 0.2535 | 0.4307 | 0.0164 | 0.0566 | 0.3810 | 0.00002 |
| Glycerol, polar | 0.0592 | 0.0872 | 0.1127 | 0.9554 | 0.8811 | 0.00002 |
| fraction | | | | | | |
| Isocitrate | 0.1099 | 0.0487 | 0.2770 | 0.2263 | 0.0270 | 0.00005 |
| Lysophosphatidylcholine | 0.5350 | 0.3624 | 0.5282 | 0.7013 | 0.3797 | 0.0005 |
| (C20:4) | | | | | | |
| 1-Hydroxy-2- | 0.0967 | 0.6466 | 0.0007 | 0.0040 | 0.1018 | 0.0224 |
| amino-(cis.trans)- | | | | | | |
| 3,5- | | | | | | |
| octadecadiene | | | | | | |
| (from sphingolipids) | | | | | | |
| Lysophosphatidylcholine | 0.7619 | 0.4577 | 0.7105 | 0.1403 | 0.0899 | 0.0008 |
| (C18:1) | | | | | | |
| Ceramide | 0.4545 | 0.9434 | 0.6958 | 0.4720 | 0.6511 | 0.0009 |
| (d18:1,C24:0) | | | | | | |
| 14,15- | 0.5545 | 0.9862 | 0.5508 | 0.6063 | 0.1835 | 0.0017 |
| Dihydroxyeicosatrienoic | | | | | | |
| acid | | | | | | |
| (C20:cis[5,8,11]3) | | | | | | |
| Lysophosphatidylcholine | 0.0620 | 0.0726 | 0.0092 | 0.0373 | 0.0017 | 0.0019 |
| (C18:2) | | | | | | |
| erythro- | 0.0221 | 0.2501 | 0.0049 | 0.0092 | 0.0999 | 0.0213 |
| Dihydrosphingosine | | | | | | |
| (d16:0) | | | | | | |
| Valine | 0.7002 | 0.7509 | 0.0767 | 0.2442 | 0.0027 | 0.7454 |
| erythro- | 0.0587 | 0.2256 | 0.0082 | 0.0042 | 0.0933 | 0.0262 |
| Sphingosine | | | | | | |
| (d18:1) | | | | | | |
| Creatine | 0.2054 | 0.5464 | 0.0028 | 0.0124 | 0.0054 | 0.2990 |
| myo-Inositol-2- | 0.1634 | 0.8405 | 0.0170 | 0.16084 | 0.0057 | 0.1657 |
| phosphate, lipid | | | | | | |
| fraction (myo- | | | | | | |
| Inositolphospho- | | | | | | |
| lipids) | | | | | | |
| Leucine | 0.8073 | 0.8729 | 0.1285 | 0.2896 | 0.0062 | 0.5191 |
| Quinic acid | 0.0638 | 0.0471 | 0.7178 | 0.2717 | 0.5571 | 0.0063 |
| Glycerol, lipid | 0.5811 | 0.3057 | 0.1937 | 0.8041 | 0.0066 | 0.2355 |
| fraction | | | | | | |
| Lysophosphatidylcholine | 0.7074 | 0.9332 | 0.6664 | 0.5475 | 0.6116 | 0.0539 |
| (C16:0) | | | | | | |
| Eicosanoic acid | 0.4622 | 0.1234 | 0.3992 | 0.2373 | 0.1169 | 0.1912 |
| (C20:0) | | | | | | |
| Octadecanoylcarnitine | 0.1400 | 0.2832 | 0.2939 | 0.4566 | 0.0090 | 0.1889 |
| Phosphatidylcholine | 0.3891 | 0.1986 | 0.5456 | 0.9870 | 0.1207 | 0.0096 |
| (C18:0,C18:1) | | | | | | |
| Serine | 0.3547 | 0.1139 | 0.7295 | 0.6446 | 0.2752 | 0.3975 |
| Erythrol | 0.1566 | 0.9651 | 0.4788 | 0.6084 | 0.0658 | 0.5354 |
| Phosphatidylcholine | 0.4074 | 0.7009 | 0.0850 | 0.4666 | 0.0107 | 0.0315 |
| (C16:0,C16:0) | | | | | | |
| Glucose-6- | 0.7220 | 0.5556 | 0.5718 | 0.4499 | 0.2620 | 0.0108 |
| phosphate | | | | | | |
| Cholesta-2,4,6- | 0.3210 | 0.0714 | 0.1192 | 0.0290 | 0.6744 | 0.0114 |
| triene | | | | | | |
| trans-4- | 0.7999 | 0.0125 | 0.7113 | 0.8789 | 0.7637 | 0.8813 |
| Hydroxyproline | | | | | | |
| Cholesterylester | 0.4332 | 0.8535 | 0.4102 | 0.1317 | 0.0847 | 0.0132 |
| C18:2 | | | | | | |
| Docosahexaenoic | 0.0753 | 0.1959 | 0.1988 | 0.0148 | 0.2146 | 0.0619 |
| acid | | | | | | |
| (C22:cis[4,7,10,13, | | | | | | |
| 16,19]6) | | | | | | |
| 4- | 0.0154 | 0.4353 | 0.1684 | 0.0915 | 0.7452 | 0.7983 |
| Hydroxysphinganine | | | | | | |
| (t18:0, Phytosphingosine), | | | | | | |
| total | | | | | | |
| 14- | 0.4215 | 0.0163 | 0.2304 | 0.2907 | 0.9797 | 0.2297 |
| Methylhexadecanoic | | | | | | |
| acid | | | | | | |
| TAG(C16:0,C18: | 0.6333 | 0.0435 | 0.5223 | 0.4349 | 0.0744 | 0.0165 |
| 1,C18:2) | | | | | | |
| Glycine | 0.1976 | 0.0172 | 0.8774 | 0.6039 | 0.5171 | 0.6700 |
| Linolenic acid | 0.1904 | 0.0187 | 0.1816 | 0.4293 | 0.4741 | 0.5606 |
| (C18:cis[9,12,15] | | | | | | |
| 3) | | | | | | |
| Behenic acid | 0.2252 | 0.6012 | 0.2851 | 0.0196 | 0.5269 | 0.8840 |
| (C22:0) | | | | | | |
| Dodecanoylcarnitine | 0.0200 | 0.0530 | 0.6094 | 0.5193 | 0.3395 | 0.7454 |
| Phosphatidylcholine | 0.0220 | 0.3751 | 0.4593 | 0.5971 | 0.5782 | 0.0602 |
| (C18:0,C18:2) | | | | | | |
| Stearic acid | 0.1556 | 0.0220 | 0.7178 | 0.0739 | 0.2986 | 0.9670 |
| (C18:0) | | | | | | |
| Palmitic acid | 0.7126 | 0.2377 | 0.4387 | 0.2951 | 0.0220 | 0.8730 |
| (C16:0) | | | | | | |
| Sorbitol | 0.1470 | 0.9546 | 0.9610 | 0.9988 | 0.8418 | 0.7335 |
| Ceramide | 0.1220 | 0.9982 | 0.8966 | 0.7781 | 0.7824 | 0.0244 |
| (d18:1,C24:1) | | | | | | |
| Hippuric acid | 0.0238 | 0.2246 | 0.7918 | 0.4783 | 0.3862 | 0.8184 |
| Cholesterol, total | 0.2317 | 0.4100 | 0.1395 | 0.0259 | 0.3014 | 0.0635 |
| Arabinose | 0.4954 | 0.7308 | 0.9971 | 0.5348 | 0.1423 | 0.4853 |
| Cortisol | 0.3106 | 0.2204 | 0.8188 | 0.3289 | 0.0274 | 0.1421 |
| Lauric acid | 0.9099 | 0.0280 | 0.9769 | 0.1951 | 0.4147 | 0.7779 |
| (C12:0) | | | | | | |
| Arachidonic acid | 0.1665 | 0.0282 | 0.8278 | 0.0291 | 0.5426 | 0.9932 |
| (C20:cis[5,8,11,14] | | | | | | |
| 4) | | | | | | |
| 5-Oxoproline | 0.0294 | 0.0430 | 0.6873 | 0.3230 | 0.9112 | 0.2098 |
| Eicosapentaenoic | 0.2718 | 0.2089 | 0.0329 | 0.8157 | 0.0396 | 0.2015 |
| acid | | | | | | |
| (C20:cis[5,8,11,14, | | | | | | |
| 17]5) | | | | | | |
| Uridine | 0.4354 | 0.6396 | 0.3684 | 0.0340 | 0.1343 | 0.4070 |
| Tricosanoic acid | 0.3465 | 0.5615 | 0.2664 | 0.0340 | 0.7199 | 0.9713 |
| (C23:0) | | | | | | |
| 4-Hydroxy-3- | 0.9079 | 0.0423 | 0.9471 | 0.4820 | 0.0376 | 0.14721 |
| methoxyphenylglycol | | | | | | |
| (HMPG) | | | | | | |
| Tetradecanoylcarnitine | 0.2697 | 0.9723 | 0.0382 | 0.7093 | 0.4093 | 0.7777 |
| O- | 0.3097 | 0.5728 | 0.7296 | 0.4777 | 0.2890 | 0.8486 |
| Phosphoethanolamine | | | | | | |
| Erucic acid | 0.9850 | 0.2471 | 0.5410 | 0.0383 | 0.0795 | 0.3758 |
| (C22:cis[13]1) | | | | | | |
| Pantothenic acid | 0.4026 | 0.9300 | 0.5499 | 0.6322 | 0.1701 | 0.7106 |
| Normetanephrine | 0.0384 | 0.5670 | 0.6951 | 0.4696 | 0.0816 | 0.2571 |
| Palmitoleic acid | 0.0385 | 0.1454 | 0.2701 | 0.8695 | 0.1387 | 0.4066 |
| (C16:cis[9]1) | | | | | | |
| Fumarate | 0.1090 | 0.4937 | 0.5381 | 0.4050 | 0.0388 | 0.3472 |
| Cholesterol, free | 0.5781 | 0.1847 | 0.7079 | 0.3969 | 0.4561 | 0.0391 |
| Cholesta-2,4- | 0.6317 | 0.0393 | 0.1280 | 0.0967 | 0.2073 | 0.4465 |
| dien | | | | | | |
| Creatinine | 0.2411 | 0.9566 | 0.0404 | 0.4151 | 0.2662 | 0.5588 |
| beta-Carotene | 0.0686 | 0.1582 | 0.1828 | 0.1295 | 0.4594 | 0.3199 |
| erythro- | 0.0470 | 0.1491 | 0.0412 | 0.0830 | 0.8873 | 0.1146 |
| Dihydrosphingosine | | | | | | |
| (d18:0) | | | | | | |
| 11,12- | 0.0456 | 0.6155 | 0.3933 | 0.4742 | 0.8462 | 0.7744 |
| Dihydro- | | | | | | |
| xyeicosatrienoic | | | | | | |
| acid | | | | | | |
| (C20:cis[5,8,14]3) | | | | | | |
| Ketoleucine | 0.2962 | 0.0418 | 0.4880 | 0.2664 | 0.4920 | 0.2340 |

TABLE 1′
Further biomarkers indicating quality issue in plasma samples
related to increased processing time of plasma samples.
Relative ratios of samples processed at indicated temperature
(21° C.) and times (5 h, 16 h) compared to control
samples as well as corresponding p-values are given.

| Temp. ° C. | 21 | 21 | 21 | 21 |
|---|---|---|---|---|
| Time | 5 | 16 | 5 | 16 |
| Biomarker (Metabolite) | Ratio relative to = | | p-value | |
| | 0 at 21° c. | | | |
| Aspartate | 0.9947 | 1.3006 | 0.95352 | 0.00486 |
| Asparagine | 0.9543 | 0.9258 | 0.00386 | 3.5E−06 |
| Aspartate to asparagine | 1.0423 | 1.4048 | 0.6399 | 0.00019 |
| intra-sample ratio | | | | |

TABLE 1a
Preferred biomarkers indicating quality issue in
plasma samples related to increased processing time
of plasma samples: Selection based on assayability.
Biomarker (Metabolite)

Glutamate
Maltose
Cysteine
Glutamate to glutamine intra-sample ratio
Glycerate
Threonic acid
Glycerol-3-phosphate, polar fraction
Glutamine
3-Phosphoglycerate (3-PGA)
Cystine

TABLE 1a′
Further preferred biomarkers indicating quality issue
in plasma samples related to increased processing time
of plasma samples: Selection based on assayability.
Biomarker (Metabolite)

Aspartate
Asparagine
Aspartate to asparagine intra-sample ratio

TABLE 1b
Preferred biomarkers indicating quality issue in
plasma samples related to increased processing time
of plasma samples: Selection based on performance.
Biomarker (Metabolite)

3,4-Dihydroxyphenylacetic acid (DOPAC)
5-Hydroxyeicosatetraenoic acid (C20:trans[6]cis[8,11,14]4) (5-
HETE)
12-Hydroxyeicosatetraenoic acid (C20:cis[5,8,10,14]4)
Glutamate
15-Hydroxyeicosatetraenoic acid (C20:cis[5,8,11,13]4)
3,4-Dihydroxyphenylglycol (DOPEG)
11-Hydroxyeicosatetraenoic acid (C20:cis[5,8,12,14]4)
3,4-Dihydroxyphenylalanine (DOPA)
8-Hydroxyeicosatetraenoic acid (C20:trans[5]cis[9,11,14]4) (8-
HETE)
Prostaglandin D2
Maltose
alpha-Ketoglutarate
Noradrenaline (Norepinephrine)
Cysteine

TABLE 1c
Preferred biomarkers indicating quality issue in plasma
samples related to increased processing time of plasma
samples: Selection based on method “GC-polar”.
Biomarker (Metabolite)

Glutamate
Maltose
alpha-Ketoglutarate
Cysteine
Glycerate
Threonic acid
Glycerol-3-phosphate, polar fraction
Pyruvate
Glutamine
3-Phosphoglycerate (3-PGA)
Glutamate to glutamine intra-sample ratio
Cystine
Alanine
Glycerol, polar fraction
Isocitrate
Valine
Leucine
Quinic acid
Serine
Erythrol
trans-4-Hydroxyproline
Glycine
Arabinose
5-Oxoproline
Fumarate
Ketoleucine

TABLE 1c′
Further preferred biomarkers indicating quality issue in
plasma samples related to increased processing time of
plasma samples: Selection based on method “GC-polar”.
Biomarker (Metabolite)

Aspartate
Asparagine
Aspartate to asparagine intra-sample ratio

TABLE 2
List of identified biomarkers indicating quality issue in plasma samples related to increased
processing time of blood samples. Relative ratios of samples processed at different
temperatures (0° C., room temperature (RT)) and times (2 h, 6 h) compared to
control samples as well as corresponding p-values are given.

| | 2 h delay | 2 h delay of | 6 h delay of | | | |
|---|---|---|---|---|---|---|
| | of blood | blood | blood processing | 2 h delay of | 2 h delay of | 6 h delay of |
| | processing | processing at | at | blood processing | blood processing | blood processing |
| | at RT | 0° C. | 0° C. | at | at | at |
| | Ratio relative | Ratio relative | Ratio relative | RT | 0° C. | 0° C. |
| Biomarker (Metabolite) | to control | to control | to control | p-value | p-value | p-value |
| Pyruvate | 0.9237 | 0.4271 | 0.2648 | 0.15266 | 2.19E−33 | 1.24E−55 |
| Hypoxanthine | 2.7667 | 1.5011 | 7.4549 | 1.3E−21 | 0.00002 | 7.93E−50 |
| Sphingadienine-1-phosphate (d18:2) | 1.5593 | 0.822 | 0.8897 | 2.55E−37 | 8.92E−12 | 0.00003 |
| Serotonin (5-HT) | 0.1498 | 0.0149 | 0.03 | 4.29E−13 | 1.22E−35 | 1E−29 |
| Ornithine | 1.3302 | 0.9597 | 0.9831 | 2.64E−28 | 0.05877 | 0.43281 |
| Thromboxane B2 | 0.5227 | 0.1244 | 0.1542 | 0.00020 | 3.06E−24 | 2.84E−21 |
| 9-Hydroxyoctadecadienoic acid (9- | 1.5235 | 0.9931 | 1.1368 | 4.14E−24 | 0.84618 | 0.00035 |
| HODE) (C18:trans[10]cis[12]2) | | | | | | |
| Sphingosine (d16:1) | 0.5092 | 0.7183 | 0.9771 | 3.38E−22 | 0.0000001 | 0.69494 |
| Sphingosine-1-phosphate (d16:1) | 1.2916 | 0.8868 | 0.9232 | 6.99E−22 | 0.000001 | 0.00077 |
| Sphingosine-1-phosphate (d18:1) | 1.2501 | 0.626 | 0.6626 | 0.000001 | 2.23E−21 | 2.42E−17 |
| Taurine | 0.4882 | 0.4477 | 0.5409 | 1.03E−17 | 1.73E−20 | 9.5E−14 |
| Oleoylcarnitine | 1.4832 | 1.0822 | 1.1527 | 3.39E−20 | 0.03942 | 0.00026 |
| Pyrophosphate (PPi) | 0.3407 | 0.2452 | 0.2659 | 7.9E−14 | 1.09E−19 | 1.85E−18 |
| O-Phosphoethanolamine | 0.3047 | 0.2261 | 0.3171 | 8.57E−14 | 7.12E−19 | 8.16E−13 |
| Sphingosine-1-phosphate (d17:1) | 1.3325 | 0.7902 | 0.8363 | 2.56E−17 | 7.49E−13 | 2.98E−08 |
| Sphingadienine (d18:2) | 0.4706 | 1.26 | 1.7673 | 7.08E−16 | 0.00673 | 2.24E−10 |
| 12-Hydroxyheptadecatrienoic acid | 0.665 | 0.2558 | 0.2722 | 0.00742 | 9.25E−16 | 4.78E−15 |
| (C17:[5.8.10]3) | | | | | | |
| Sphingosine (d18:1) | 0.6479 | 0.4592 | 0.7557 | 0.00001 | 1.53E−13 | 0.00482 |
| Sphinganine-1-phosphate (d18:0) | 1.1352 | 0.5747 | 0.5884 | 0.06551 | 2.14E−13 | 1.52E−12 |
| Hypotaurine | 0.5854 | 0.5478 | 0.6259 | 2.5E−11 | 7E−13 | 4.95E−09 |
| 3,4-Dihydroxyphenylglycol (DOPEG) | 0.7217 | 0.9493 | 0.9171 | 7.23E−13 | 0.21650 | 0.04371 |
| Maltose | 0.3386 | 0.2442 | 0.2678 | 1.08E−09 | 8.93E−13 | 1.67E−12 |
| Sphinganine (d18:0) | 0.7282 | 0.5513 | 0.6397 | 0.00005 | 3.06E−12 | 0.0000001 |
| Noradrenaline (Norepinephrine) | 0.7561 | 0.805 | 0.7926 | 3.96E−11 | 0.0000001 | 3.41E−08 |
| Dopamine | 0.6535 | 0.719 | 0.6487 | 4.77E−10 | 0.000001 | 4.48E−10 |
| Glycerol-3-phosphate, polar fraction | 0.7529 | 0.5591 | 0.6369 | 0.00121 | 1.73E−09 | 0.000002 |
| Nicotinamide | 0.7927 | 0.5182 | 0.7134 | 0.02712 | 3.79E−09 | 0.00167 |
| Glutamate | 0.8161 | 0.6467 | 0.7016 | 0.01224 | 0.0000003 | 0.00002 |
| 13-Hydroxyoctadecadienoic acid (13- | 1.1475 | 0.9695 | 1.0087 | 0.0000005 | 0.24740 | 0.74316 |
| HODE) (C18:cis[9]trans[11]2) | | | | | | |
| Octadecanoylcarnitine | 1.2053 | 0.9566 | 1.0059 | 0.000001 | 0.23855 | 0.87445 |
| 12-Hydroxyeicosatetraenoic acid | 0.4754 | 0.3905 | 0.6592 | 0.00007 | 0.000001 | 0.02285 |
| (C20:cis[5,8,10,14]4) | | | | | | |
| Glycerol, polar fraction | 0.7723 | 0.8141 | 0.8409 | 0.000002 | 0.00016 | 0.00135 |
| Maltotriose | 0.2299 | 0.2835 | 0.1744 | 0.00002 | 0.04029 | 0.000002 |
| Phosphate (inorganic and from organic | 0.8632 | 0.9255 | 0.9475 | 0.00001 | 0.01664 | 0.09375 |
| phosphates) | | | | | | |
| myo-Inositol | 0.9292 | 0.897 | 0.9109 | 0.00285 | 0.00002 | 0.00021 |
| Lactaldehyde | 0.9018 | 0.9116 | 0.8167 | 0.02417 | 0.04622 | 0.00002 |
| 11-Hydroxyeicosatetraenoic acid | 0.9689 | 0.7766 | 0.8703 | 0.5845 | 0.00003 | 0.01703 |
| (C20:cis[5,8,12,14]4) | | | | | | |
| Pentoses | 1.4465 | 1.037 | 1.1263 | 0.00005 | 0.68630 | 0.18716 |
| 3,4-Dihydroxyphenylacetic acid | 0.8335 | 1.0348 | 0.9723 | 0.00008 | 0.44968 | 0.54142 |
| (DOPAC) | | | | | | |
| Phosphatidylcholine (C18:0,C22:6) | 1.0627 | 1.0369 | 1.0295 | 0.00016 | 0.02452 | 0.07050 |
| 5-Hydroxyeicosatetraenoic acid | 1.25 | 1.069 | 1.1743 | 0.00024 | 0.28548 | 0.00726 |
| (C20:trans[6]cis[8,11,14]4) (5-HETE) | | | | | | |
| Hexadecanoylcarnitine | 1.158 | 1.0667 | 1.0583 | 0.00038 | 0.11748 | 0.16871 |
| Fructose | 1.339 | 0.9637 | 1.055 | 0.00061 | 0.66309 | 0.53300 |
| Hexadecenoylcarnitine | 1.1848 | 1.0416 | 1.0441 | 0.00062 | 0.40989 | 0.38282 |
| Tetradecanol | 0.6988 | 0.7594 | 0.7233 | 0.00071 | 0.00974 | 0.00244 |
| 3-Hydroxyindole | 0.9298 | 0.8992 | 0.825 | 0.23476 | 0.08805 | 0.00222 |
| Lysophosphatidylcholine (C17:0) | 1.1257 | 1.0411 | 0.995 | 0.00277 | 0.31017 | 0.89922 |
| Glycerate | 1.2012 | 0.9432 | 1.0026 | 0.00307 | 0.34622 | 0.96685 |
| Mannose | 0.8683 | 0.9931 | 0.9634 | 0.00312 | 0.88482 | 0.43580 |
| 15-Hydroxyeicosatetraenoic acid | 0.9961 | 0.8492 | 0.933 | 0.94503 | 0.00465 | 0.21786 |
| (C20:cis[5,8,11,13]4) | | | | | | |
| Heptadecanoic acid (C17:0) | 1.0989 | 1.1965 | 1.0861 | 0.12855 | 0.00471 | 0.18878 |
| Adrenaline (Epinephrine) | 0.8726 | 0.923 | 0.9118 | 0.00473 | 0.09420 | 0.05788 |
| Urea | 1.0775 | 1.19 | 1.0056 | 0.21818 | 0.00507 | 0.92731 |
| 2-Hydroxybutyrate | 1.0187 | 1.0006 | 1.051 | 0.28446 | 0.97486 | 0.00511 |
| Tryptophan | 1.0488 | 1.0314 | 1.0745 | 0.05893 | 0.22582 | 0.00526 |
| Glucose-6-phosphate | 0.6361 | 0.6845 | 0.8752 | 0.00657 | 0.02421 | 0.42498 |
| Fumarate | 1.0814 | 1.053 | 1.0533 | 0.00695 | 0.07702 | 0.07582 |
| 14-Methylhexadecanoic acid | 1.1657 | 1.2851 | 1.1639 | 0.10751 | 0.00989 | 0.11618 |
| Tricosanoic acid (C23:0) | 1.1851 | 1.1367 | 1.0853 | 0.01049 | 0.05600 | 0.22039 |
| Isopalmitic acid (C16:0) | 1.1852 | 1.2492 | 1.1259 | 0.05856 | 0.01496 | 0.19173 |
| Sphingomyelin (d18:2,C16:0) | 1.042 | 1.0744 | 1.0896 | 0.23622 | 0.04262 | 0.01562 |
| Normetanephrine | 1.5631 | 1.4136 | 1.671 | 0.03618 | 0.09433 | 0.01876 |
| Phosphatidylcholine (C16:0,C16:0) | 0.9594 | 0.957 | 0.9872 | 0.02556 | 0.01970 | 0.49183 |
| Leucine | 1.0337 | 0.9874 | 1.018 | 0.02030 | 0.37767 | 0.21504 |
| Myristic acid (C14:0) | 1.1497 | 1.3714 | 1.1594 | 0.30410 | 0.02259 | 0.28267 |
| Phosphatidylcholine (C16:0,C22:6) | 1.0085 | 1.003 | 1.0231 | 0.38898 | 0.76130 | 0.02340 |
| Glycerol, lipid fraction | 1.1213 | 1.3585 | 1.2168 | 0.38747 | 0.02350 | 0.14506 |
| Sphingomyelin (d18:2,C18:0) | 1.0361 | 1.0291 | 1.0488 | 0.08850 | 0.17437 | 0.02453 |
| Galactose, lipid fraction | 1.0349 | 1.12 | 1.0485 | 0.48718 | 0.02485 | 0.34531 |
| Cholesterylester C18:1 | 1.1066 | 1.0118 | 1.0416 | 0.02585 | 0.79819 | 0.37405 |
| Ketoleucine | 0.914 | 0.9233 | 0.9535 | 0.02827 | 0.05448 | 0.24911 |
| Proline | 1.0305 | 0.9762 | 0.9956 | 0.03080 | 0.08718 | 0.75148 |
| Malate | 1.0019 | 0.8841 | 0.955 | 0.97231 | 0.03143 | 0.41828 |
| Phosphatidylcholine (C18:1,C18:2) | 0.9943 | 0.9847 | 0.9964 | 0.42820 | 0.03502 | 0.62105 |
| Erythrol | 0.9299 | 0.9429 | 0.9359 | 0.03781 | 0.09729 | 0.06182 |
| Metanephrine | 0.8596 | 1.0715 | 1.016 | 0.03957 | 0.34475 | 0.83059 |
| beta-Alanine | 0.9175 | 0.9115 | 0.9143 | 0.05350 | 0.04064 | 0.04750 |
| Oleic acid (C18:cis[9]1) | 1.0746 | 1.1649 | 1.1293 | 0.32571 | 0.04072 | 0.10224 |
| Histamine | 0.7312 | 0.6596 | 0.9459 | 0.11959 | 0.04218 | 0.78472 |
| Stearic acid (C18:0) | 1.0579 | 1.1377 | 1.091 | 0.36650 | 0.04229 | 0.16925 |
| Cortisol | 1.0981 | 1.028 | 1.0074 | 0.04229 | 0.55240 | 0.87411 |
| Cholesta-2,4-dien | 1.1008 | 1.1555 | 1.1043 | 0.18343 | 0.04918 | 0.17562 |
| Dodecanoylcarnitine | 1.0956 | 0.9808 | 0.9924 | 0.04946 | 0.67993 | 0.87007 |
| Arginine | 0.7668 | 1.114 | 1.2211 | 0.04998 | 0.42958 | 0.14494 |
| Threonine | 1.0299 | 0.9796 | 1.004 | 0.05161 | 0.17672 | 0.79411 |
| Tetradecanoylcarnitine | 1.0992 | 1.014 | 1.0565 | 0.05173 | 0.77724 | 0.26320 |
| threo-Sphingosine (d18:1) | 1.0683 | 1.1076 | 1.037 | 0.20142 | 0.05210 | 0.48742 |
| Glucose-1-phosphate | 0.9069 | 1.0453 | 0.9625 | 0.05441 | 0.38799 | 0.45694 |
| Lignoceric acid (C24:0) | 1.1235 | 1.1003 | 1.0656 | 0.05493 | 0.11977 | 0.30036 |
| Palmitic acid (C16:0) | 1.1062 | 1.1843 | 1.1749 | 0.24553 | 0.05591 | 0.06837 |
| Alanine | 1.0152 | 0.9729 | 0.984 | 0.29066 | 0.05845 | 0.26539 |
| TAG (C18:1,C18:2,C18:3) | 1.0846 | 1.108 | 1.0715 | 0.12892 | 0.05902 | 0.20244 |
| Pantothenic acid | 0.9763 | 1.1437 | 1.1629 | 0.76165 | 0.09492 | 0.06090 |
| Eicosanoic acid (C20:0) | 1.0977 | 1.0984 | 1.0409 | 0.06207 | 0.06402 | 0.42742 |
| 8,9-Dihydroxyeicosatrienoic acid | 1.106 | 0.9355 | 1.0223 | 0.06344 | 0.22548 | 0.68265 |
| (C20:cis[5,11,14]3) | | | | | | |
| Mannosamine | 0.5836 | 0.9013 | 0.6514 | 0.06397 | 0.73119 | 0.15753 |
| Sulfate | 0.986 | 1.0348 | 1.1736 | 0.87004 | 0.69043 | 0.06503 |
| Indole-3-lactic acid | 0.9908 | 1.0532 | 1.063 | 0.78391 | 0.12905 | 0.07379 |
| Lysophosphatidylcholine (C18:0) | 1.0928 | 0.9508 | 0.9584 | 0.07657 | 0.31937 | 0.40193 |
| erythro-Sphingosine-1-phosphate | 1.1413 | 1.1591 | 1.1143 | 0.10845 | 0.07737 | 0.19434 |
| (d18:1) | | | | | | |
| Lysophosphatidylcholine (C18:1) | 1.0636 | 1.0434 | 1.0372 | 0.07878 | 0.23174 | 0.30288 |
| Methionine | 0.9688 | 0.9874 | 1.0079 | 0.08130 | 0.48977 | 0.66801 |
| Ceramide (d18:1,C24:0) | 1.0744 | 1.0334 | 1.0597 | 0.08431 | 0.43454 | 0.16827 |
| Dehydroepiandrosterone sulfate | 0.772 | 0.9693 | 1.1363 | 0.08549 | 0.83731 | 0.40091 |
| myo-Inositol, lipid fraction | 0.9867 | 1.1206 | 1.0446 | 0.83842 | 0.08978 | 0.51457 |
| Phosphatidylcholine (C16:0,C20:4) | 0.9963 | 0.9941 | 0.9876 | 0.61100 | 0.41906 | 0.09029 |
| beta-Carotene | 1.0931 | 1.0473 | 1.0573 | 0.09050 | 0.38478 | 0.29454 |
| erythro-Sphingosine (d18:1) | 1.0924 | 1.105 | 1.1035 | 0.12858 | 0.09086 | 0.09522 |
| erythro-Dihydrosphingosine (d18:0) | 1.1356 | 1.1332 | 1.084 | 0.09463 | 0.10501 | 0.29465 |
| Behenic acid (C22:0) | 1.0876 | 1.0846 | 1.0145 | 0.09468 | 0.11085 | 0.77674 |
| Lysophosphatidylcholine (C16:0) | 0.8817 | 0.9514 | 0.9404 | 0.09669 | 0.51559 | 0.42225 |
| Isocitrate | 1.0348 | 0.9339 | 0.9955 | 0.39852 | 0.09768 | 0.91207 |
| Linoleic acid (C18:cis[9,12]2) | 1.075 | 1.0875 | 1.0998 | 0.20838 | 0.15030 | 0.10334 |
| Palmitoleic acid (C16:cis[9]1) | 1.0944 | 1.1959 | 1.1283 | 0.40369 | 0.10342 | 0.27082 |
| Cholesterylester C20:4 | 1.0347 | 1.0711 | 1.097 | 0.54281 | 0.22752 | 0.10450 |
| 8-Hydroxyeicosatetraenoic acid | 0.9253 | 0.9442 | 0.9238 | 0.11412 | 0.25663 | 0.10705 |
| (C20:trans[5]cis[9,11,14]4) (8-HETE) | | | | | | |
| Sphingomyelin (d18:1,C24:0) | 1.0173 | 0.9909 | 1.0313 | 0.36450 | 0.63312 | 0.10965 |
| Phosphate, lipid fraction | 1.1007 | 1.0555 | 1.0542 | 0.11019 | 0.37415 | 0.39381 |
| 3,4-Dihydroxyphenylalanine (DOPA) | 1.039 | 0.9899 | 0.9978 | 0.12304 | 0.68152 | 0.92883 |
| Glucuronic acid | 1.0496 | 1.1846 | 1.3333 | 0.79431 | 0.36845 | 0.12752 |
| Phosphatidylcholine (C18:0,C18:1) | 1.0019 | 1.0049 | 1.0174 | 0.86480 | 0.66808 | 0.12938 |
| conjugated Linoleic acid | 1.0564 | 1.145 | 1.1016 | 0.53234 | 0.13002 | 0.27849 |
| (C18:trans[9,11]2) | | | | | | |
| Serine | 1.0242 | 0.9789 | 0.9916 | 0.13796 | 0.19365 | 0.60750 |
| Glycochenodeoxycholic acid | 0.9628 | 0.9946 | 0.8593 | 0.71030 | 0.95798 | 0.14467 |
| Tyrosine | 1.0037 | 0.9709 | 0.9828 | 0.85400 | 0.14989 | 0.39663 |
| Docosapentaenoic acid | 1.0602 | 1.1258 | 0.9985 | 0.47008 | 0.15003 | 0.98516 |
| (C22:cis[7,10,13,16,19]5) | | | | | | |
| 1-Hydroxy-2-amino-(cis.trans)-3,5- | 1.116 | 1.0857 | 1.0557 | 0.15042 | 0.29503 | 0.48293 |
| octadecadiene (from sphingolipids) | | | | | | |
| Cystine | 0.8574 | 0.9021 | 0.8427 | 0.19281 | 0.38913 | 0.15337 |
| Glycine | 1.0008 | 0.9809 | 0.998 | 0.95376 | 0.15564 | 0.88406 |
| dihomo-gamma-Linolenic acid | 1.044 | 1.097 | 1.1064 | 0.54027 | 0.19479 | 0.15694 |
| (C20:cis[8,11,14]3) | | | | | | |
| Serine, lipid fraction | 0.8848 | 1.017 | 0.8047 | 0.42068 | 0.91387 | 0.15908 |
| Linolenic acid (C18:cis[9,12,15]3) | 1.1042 | 1.1415 | 1.1195 | 0.28425 | 0.15920 | 0.22954 |
| Cholesterol, total | 1.0572 | 1.0988 | 1.0499 | 0.40269 | 0.16271 | 0.46960 |
| Eicosapentaenoic acid | 1.1052 | 1.1788 | 1.0189 | 0.39073 | 0.16456 | 0.87373 |
| (C20:cis[5,8,11,14,17]5) | | | | | | |
| Lysophosphatidylcholine (C20:4) | 1.0295 | 1.0552 | 1.0435 | 0.45016 | 0.16869 | 0.27539 |
| Indole-3-acetic acid | 0.9545 | 0.9517 | 1.0083 | 0.19465 | 0.17451 | 0.81940 |
| Citrulline | 1.0645 | 1.1421 | 1.1693 | 0.58242 | 0.24991 | 0.17577 |
| Lysine | 1.0192 | 0.968 | 0.9851 | 0.42287 | 0.17659 | 0.53101 |
| Citrate | 1.0066 | 0.9713 | 0.9631 | 0.81242 | 0.29980 | 0.18107 |
| Phosphatidylcholine (C18:0,C18:2) | 0.9932 | 0.9944 | 0.9885 | 0.42737 | 0.51476 | 0.18127 |
| Glycerol phosphate, lipid fraction | 1.0643 | 1.1205 | 1.1009 | 0.47157 | 0.19574 | 0.27390 |
| Phosphatidylcholine (C16:0,C20:5) | 1.0114 | 1.0044 | 1.0277 | 0.58612 | 0.83491 | 0.19754 |

TABLE 2′
Further biomarkers indicating quality issue in plasma samples related to increased
processing time of blood samples. Relative ratios of samples processed at different
temperatures (0° C., room temperature (RT)) and times (2 h, 6 h) compared to
control samples as well as corresponding p-values are given.

| | 2 h delay | 2 h delay of | 6 h delay of | | | |
|---|---|---|---|---|---|---|
| | of blood | blood | blood processing | 2 h delay of | | 6 h delay of |
| | processing | processing at | at | blood processing | 2 h delay of | blood processing |
| | at RT | 0° C. | 0° C. | at | blood processing | at |
| | Ratio relative | Ratio relative | Ratio relative | RT | at 0° C. | 0° C. |
| Biomarker (Metabolite) | to control | to control | to control | p-value | p-value | p-value |
| Glutamate to glutamine | 0.5571 | 0.4656 | 0.4998 | 0.000396 | 5.19E−06 | 3.149E−05 |
| intra-sample ratio | | | | | | |
| Threonic acid | 0.9674 | 0.7984 | 0.8585 | 0.543051 | 6.11044E−05 | 0.0057723 |
| Asparagine | 1.0158 | 0.9475 | 0.9747 | 0.326067 | 0.000913218 | 0.1093793 |
| Aspartate to asparagine | 0.6481 | 0.6478 | 0.7402 | 2.64E−06 | 2.5852E−06 | 0.0008766 |
| intra-sample ratio | | | | | | |
| Aspartate | 0.6583 | 0.6137 | 0.7214 | 1.16E−05 | 4.26728E−07 | 0.0005178 |
| Cysteine | 0.9609 | 0.9474 | 0.9949 | 0.097743 | 0.025652048 | 0.8322395 |
| Ornithine to Arginine | 1.7765 | 0.951 | 1.1244 | 8.42E−19 | 0.36660178 | 0.036343 |
| intra-sample ratio | | | | | | |
| Ribose | 0.8909 | 0.9498 | 0.9013 | 0.049903 | 0.378629357 | 0.077369 |
| 3-Phosphoglycerate | 0.1902 | 0.1622 | 0.2693 | 1.72E−18 | 1.61604E−20 | 2.744E−13 |
| (3-PGA) | | | | | | |

TABLE 2a
Preferred biomarkers indicating quality issue in
plasma samples related to increased processing time
of blood samples: Selection based on assayability.
Biomarker (Metabolite)

Hypoxanthine
Ornithine
Taurine
Maltose
Glycerol-3-phosphate, polar fraction
Glutamate
Glycerate
Arginine
Cystine
Citrate

TABLE 2a′
Further preferred biomarkers indicating quality issue
in plasma samples related to increased processing time
of blood samples: Selection based on assayability.
Biomarker (Metabolite)

Glutamate to glutamine intra-sample ratio
Threonic acid
Asparagine
Aspartate to asparagine intra-sample ratio
Aspartate
Cysteine
Ornithine to Arginine intra-sample ratio
Ribose
3-Phosphoglycerate (3-PGA)

TABLE 2b
Preferred biomarkers indicating quality issue in
plasma samples related to increased processing time
of blood samples: Selection based on performance.
Biomarker (Metabolite)

Hypoxanthine
Sphingadienine-1-phosphate (d18:2)
Ornithine
Thromboxane B2
9-Hydroxyoctadecadienoic acid (9-HODE) (C18:trans[10]cis[12]2)
Sphingosine (d16:1)
Sphingosine-1-phosphate (d16:1)
Sphingosine-1-phosphate (d18:1)
Taurine
Oleoylcarnitine
Pyrophosphate (PPi)
Sphingosine-1-phosphate (d17:1)
Sphingadienine (d18:2)
Sphingosine (d18:1)
Sphinganine-1-phosphate (d18:0)

TABLE 2b′
A further preferred biomarker indicating quality issue in
plasma samples related to increased processing time
of blood samples: Selection based on performance.
Biomarker (Metabolite)

Ornithine to Arginine intra-sample ratio

TABLE 2c
Preferred biomarkers indicating quality issue in plasma
samples related to increased processing time of blood
samples: Selection based on method “GC-polar”.
Biomarker (Metabolite)

Pyruvate
Hypoxanthine
Ornithine
Taurine
Pyrophosphate (PPi)
Hypotaurine
Maltose
Glycerol-3-phosphate, polar fraction
Glutamate
Glycerol, polar fraction
Maltotriose
Phosphate (inorganic and from organic phosphates)
myo-Inositol
Fructose
3-Hydroxyindole
Glycerate
Mannose
2-Hydroxybutyrate
Tryptophan
Fumarate
Leucine
Ketoleucine
Proline
Malate
Erythrol
beta-Alanine
Threonine
Glucose-1-phosphate
Alanine
Mannosamine
Sulfate
Methionine
Isocitrate
Serine
Tyrosine
Cystine
Glycine
Indole-3-acetic acid
Lysine
Citrate

TABLE 2c′
Further preferred biomarkers indicating quality issue in
plasma samples related to increased processing time
of blood samples: Selection based on method “GC-polar”.
Biomarker (Metabolite)

Glutamate to glutamine intra-sample ratio
Threonic acid
Asparagine
Aspartate to asparagine intra-sample ratio
Aspartate
Cysteine
Ornithine to Arginine intra-sample ratio
Ribose
3-Phosphoglycerate (3-PGA)

TABLE 3
List of identified biomarkers indicating quality issue in plasma samples related to hemolysis

| | Hemolysis | Hemolysis | | |
|---|---|---|---|---|
| | Grade 1 | Grade 2 | Hemolysis | Hemolysis |
| | Ratio relative | Ratio relative | Grade 1 | Grade 2 |
| Biomarker (Metabolite) | to control | to control | p-value | p-value |
| Sphingadienine (d18:2) | 0.4019 | 0.4411 | 2.12E−21 | 2.6E−18 |
| Sphingosine (d18:1) | 0.3429 | 0.3737 | 3.08E−21 | 3.32E−19 |
| Sphingosine-1-phosphate | 0.6527 | 0.6737 | 1.13E−18 | 1.16E−16 |
| (d18:1) | | | | |
| Sphingosine (d16:1) | 0.5604 | 0.5655 | 3.36E−18 | 3.68E−18 |
| Thromboxane B2 | 0.225 | 0.2155 | 1.4E−14 | 2.68E−16 |
| Taurine | 0.5131 | 0.5457 | 6.47E−16 | 9.51E−14 |
| Sphinganine (d18:0) | 0.5116 | 0.6261 | 1.19E−15 | 4.31E−09 |
| Sphinganine-1-phosphate | 0.5787 | 0.6317 | 2.01E−13 | 2.79E−10 |
| (d18:0) | | | | |
| Pyrophosphate (PPi) | 0.3763 | 0.5027 | 6.26E−12 | 5.56E−07 |
| Serotonin (5-HT) | 0.164 | 0.2015 | 7.09E−12 | 4.13E−10 |
| Hypotaurine | 0.5699 | 0.5915 | 1.28E−11 | 9.76E−11 |
| Sphingosine-1-phosphate | 0.8046 | 0.8052 | 2.24E−11 | 2.54E−11 |
| (d17:1) | | | | |
| Sphingadienine-1- | 0.838 | 0.8346 | 4.83E−10 | 2.1E−10 |
| phosphate (d18:2) | | | | |
| O-Phosphoethanolamine | 0.4002 | 0.3749 | 2.94E−09 | 2.76E−10 |
| 12- | 0.3796 | 0.3876 | 3.73E−09 | 1.68E−09 |
| Hydroxyheptadecatrienoic | | | | |
| acid (C17:[5,8,10]3) | | | | |
| Maltose | 0.3527 | 0.3748 | 3.83E−09 | 3.63E−08 |
| 3-Hydroxyindole | 0.8298 | 0.7002 | 0.00298768 | 2.64E−08 |
| Noradrenaline (Norepinephrine) | 0.8309 | 0.8012 | 0.00000573 | 5.62E−08 |
| Sphingosine-1-phosphate | 0.919 | 0.886 | 0.0003221 | 4.37E−07 |
| (d16:1) | | | | |
| 11- | 0.7502 | 0.8052 | 0.00000311 | 0.00019451 |
| Hydroxyeicosatetraenoic | | | | |
| acid (C20:cis[5,8,12,14]4) | | | | |
| 12- | 0.4219 | 0.4584 | 0.00000811 | 0.000023 |
| Hydroxyeicosatetraenoic | | | | |
| acid (C20:cis[5,8,10,14]4) | | | | |
| Oleoylcarnitine | 1.0225 | 1.1838 | 0.55456842 | 0.0000126 |
| Maltotriose | 0.2636 | 0.3016 | 0.000032 | 0.0000703 |
| Glycerol-3-phosphate, polar | 0.6787 | 0.7846 | 0.0000431 | 0.00793678 |
| fraction | | | | |
| Glutamate | 0.7363 | 0.8231 | 0.00019055 | 0.01630403 |
| Octadecanoylcarnitine | 1.0271 | 1.1462 | 0.4706113 | 0.00029742 |
| myo-Inositol | 0.9156 | 0.9373 | 0.00037287 | 0.00840466 |
| Ceramide (d18:1,C24:0) | 1.0635 | 1.1606 | 0.13800348 | 0.00040892 |
| Glycerol, polar fraction | 0.854 | 0.8263 | 0.00301127 | 0.00043557 |
| Nicotinamide | 0.6885 | 0.9449 | 0.00044737 | 0.58722816 |
| Myristic acid (C14:0) | 1.2134 | 1.6207 | 0.15459447 | 0.00046693 |
| Indole-3-acetic acid | 0.9195 | 0.8804 | 0.02020044 | 0.00048032 |
| 14-Methylhexadecanoic | 1.2253 | 1.3952 | 0.03345615 | 0.00056661 |
| acid | | | | |
| Dopamine | 0.8012 | 0.8306 | 0.00071042 | 0.00384527 |
| Heptadecanoic acid | 1.159 | 1.2272 | 0.01793189 | 0.00111644 |
| (C17:0) | | | | |
| Sulfate | 1.1761 | 1.3153 | 0.06549211 | 0.00128012 |
| Tetradecanol | 0.7158 | 0.7436 | 0.00155293 | 0.00491137 |
| Hexadecanoylcarnitine | 0.9909 | 1.1379 | 0.82185551 | 0.00167649 |
| Isopalmitic acid (C16:0) | 1.1513 | 1.3289 | 0.11604202 | 0.00170911 |
| Stearic acid (C18:0) | 1.1086 | 1.2174 | 0.09912423 | 0.00184317 |
| Tricosanoic acid (C23:0) | 1.0906 | 1.2294 | 0.18823507 | 0.0019529 |
| Fumarate | 1.07 | 1.0915 | 0.01924515 | 0.00259049 |
| Glycerol. lipid fraction | 1.3653 | 1.4929 | 0.01960141 | 0.0028074 |
| Palmitic acid (C16:0) | 1.1503 | 1.2882 | 0.10800082 | 0.00393825 |
| 1-Hydroxy-2-amino- | 1.0872 | 1.2479 | 0.27273192 | 0.00458857 |
| (cis.trans)-3,5- | | | | |
| octadecadiene (from | | | | |
| sphingolipids) | | | | |
| Ceramide (d18:1,C24:1) | 1.0138 | 1.1189 | 0.73842126 | 0.00686432 |
| conjugated Linoleic acid | 1.111 | 1.2712 | 0.23188405 | 0.0068945 |
| (C18:trans[9,11]2) | | | | |
| erythro- | 1.1077 | 1.2274 | 0.17823928 | 0.00746165 |
| Dihydrosphingosine | | | | |
| (d18:0) | | | | |
| beta-Alanine | 0.8878 | 0.9446 | 0.00788311 | 0.19957198 |
| Lignoceric acid (C24:0) | 1.0478 | 1.1736 | 0.43961659 | 0.00863095 |
| 15- | 0.8592 | 0.8842 | 0.00957951 | 0.02739644 |
| Hydroxyeicosatetraenoic | | | | |
| acid (C20:cis[5,8,11,13]4) | | | | |
| Phosphate, lipid fraction | 1.0363 | 1.1662 | 0.57151128 | 0.01229448 |
| Docosapentaenoic acid | 1.1662 | 1.2291 | 0.05870958 | 0.01277854 |
| (C22:cis[7,10,13,16,19]5) | | | | |
| Palmitoleic acid | 1.0239 | 1.3109 | 0.82698083 | 0.01289247 |
| (C16:cis[9]1) | | | | |
| erythro-Sphingosine | 1.0567 | 1.1556 | 0.34213421 | 0.01342472 |
| (d18:1) | | | | |
| Uric acid | 0.9763 | 0.9373 | 0.36033477 | 0.01425087 |
| Phosphatidylcholine | 0.9825 | 0.9903 | 0.01502642 | 0.17572158 |
| (C18:1,C18:2) | | | | |
| Sphingomyelin | 0.9957 | 1.0472 | 0.81826216 | 0.01563259 |
| (d18:1,C24:0) | | | | |
| Sphingomyelin | 1.0447 | 1.0504 | 0.03631959 | 0.01867204 |
| (d18:2,C18:0) | | | | |
| erythro-Sphingosine-1- | 1.0761 | 1.2111 | 0.37192322 | 0.0205107 |
| phosphate (d18:1) | | | | |
| threo-Sphingosine (d18:1) | 1.0894 | 1.1271 | 0.09822827 | 0.02133293 |
| Cholesterol, total | 1.0266 | 1.1651 | 0.69207227 | 0.02232955 |
| Oleic acid (C18:cis[9]1) | 1.1678 | 1.1826 | 0.03499891 | 0.02277323 |
| Phosphatidylcholine | 1.0265 | 1.0367 | 0.097723 | 0.02300177 |
| (C18:0,C22:6) | | | | |
| Eicosanoic acid (C20:0) | 1.0794 | 1.1178 | 0.12557461 | 0.02619107 |
| Linoleic acid | 1.0981 | 1.1368 | 0.10397714 | 0.02635711 |
| (C18:cis[9,12]2) | | | | |
| Glycerol phosphate, lipid | 1.0431 | 1.2103 | 0.62589921 | 0.02841455 |
| fraction | | | | |
| Urea | 1.1414 | 1.0383 | 0.02989533 | 0.53496231 |
| Cortisol | 1.1052 | 1.0942 | 0.03021237 | 0.05069531 |
| Normetanephrine | 1.5755 | 1.4654 | 0.03120381 | 0.06936646 |
| Behenic acid (C22:0) | 1.0262 | 1.1143 | 0.60468832 | 0.03171684 |
| Eicosapentaenoic acid | 1.1364 | 1.2846 | 0.27285073 | 0.03255399 |
| (C20:cis[5,8,11,14,17]5) | | | | |
| Lysophosphatidylcholine | 1.0299 | 1.0876 | 0.45122371 | 0.032632 |
| (C17:0) | | | | |
| erythro- | 1.0784 | 1.2411 | 0.4543911 | 0.03338574 |
| Dihydrosphingosine | | | | |
| (d16:0) | | | | |
| TAG (C18:1,C18:2,C18:3) | 1.0782 | 1.119 | 0.15875664 | 0.03605895 |
| Galactose, lipid fraction | 1.06 | 1.1075 | 0.23887084 | 0.03995467 |
| Fructosamine | 1.1491 | 1.3966 | 0.42463234 | 0.04560203 |
| 3,4- | 0.9626 | 0.953 | 0.12442433 | 0.04956619 |
| Dihydroxyphenylalanine | | | | |
| (DOPA) | | | | |
| 5-O-Methylsphingosine | 1.0762 | 1.2132 | 0.45644765 | 0.05099612 |
| (d16:1) | | | | |
| Cholesterylester C18:1 | 1.055 | 1.0924 | 0.23648261 | 0.05144797 |
| Glycolate | 1.0642 | 1.1807 | 0.4672714 | 0.05342104 |
| gamma-Linolenic acid | 1.0068 | 1.1914 | 0.94076475 | 0.05462627 |
| (C18:cis[6,9,12]3) | | | | |
| Coenzyme Q9 | 0.943 | 1.1275 | 0.35797377 | 0.06102849 |
| Linolenic acid | 1.1898 | 1.1695 | 0.06138493 | 0.0915892 |
| (C18:cis[9,12,15]3) | | | | |
| Phosphatidylcholine | 0.966 | 0.9908 | 0.06217669 | 0.61613526 |
| (C16:0,C16:0) | | | | |
| Cholesta-2,4-dien | 1.0984 | 1.1445 | 0.19358928 | 0.06224262 |
| Sarcosine | 0.9944 | 1.045 | 0.81212785 | 0.06572449 |
| 1,5-Anhydrosorbitol | 0.9612 | 0.9747 | 0.06700142 | 0.2346823 |
| Hexadecenoylcarnitine | 1.0014 | 1.0937 | 0.97694551 | 0.0671034 |
| Nervonic acid | 1.0423 | 1.1238 | 0.51907982 | 0.07057989 |
| (C24:cis[15]1) | | | | |
| Arachidonic acid | 0.9927 | 1.1798 | 0.93647679 | 0.07276058 |
| (C20:cis[5,8,11,14]4) | | | | |
| Alanine | 0.9748 | 0.99 | 0.07453226 | 0.48203673 |
| dihomo-gamma-Linolenic | 0.9825 | 1.1333 | 0.80140762 | 0.07605609 |
| acid (C20:cis[8,11,14]3) | | | | |
| Uridine | 1.1171 | 1.0338 | 0.07615628 | 0.59274993 |
| Sphingomyelin | 1.0086 | 1.0556 | 0.77695655 | 0.07625166 |
| (d18:1,C23:0) | | | | |
| Choline plasmalogen | 0.9653 | 0.9829 | 0.07748573 | 0.38752846 |
| (C18,C20:4) | | | | |
| Sphingomyelin | 1.0623 | 0.9888 | 0.08278466 | 0.74457271 |
| (d18:2,C16:0) | | | | |
| Malate | 0.9073 | 0.9588 | 0.08418822 | 0.45374635 |
| Phosphate (inorganic and | 0.9485 | 0.9467 | 0.09543672 | 0.08465536 |
| from organic phosphates) | | | | |
| 2-Hydroxybutyrate | 0.9712 | 0.9796 | 0.09179728 | 0.23454674 |
| Glycerate | 0.9027 | 1.0902 | 0.09508601 | 0.15855904 |
| 8-Hydroxyeicosatetraenoic | 0.9199 | 0.9533 | 0.09979566 | 0.32281559 |
| acid | | | | |
| (C20:trans[5]cis[9,11,14]4) | | | | |
| (8-HETE) | | | | |
| Cystine | 0.8768 | 0.8232 | 0.26539304 | 0.0999352 |
| Cholesterylester C18:2 | 1.0255 | 1.0673 | 0.52349069 | 0.1005164 |
| Glucose-6-phosphate | 1.3154 | 1.3046 | 0.10189345 | 0.11247861 |
| Histamine | 0.7212 | 0.7335 | 0.10430583 | 0.11814385 |
| Pseudouridine | 1.0375 | 1.0558 | 0.27072826 | 0.10448217 |
| Threitol | 1.1419 | 0.9894 | 0.10454186 | 0.89704377 |
| Lysophosphatidylcholine | 0.9903 | 1.0644 | 0.79911675 | 0.10530508 |
| (C20:4) | | | | |
| Isocitrate | 0.9367 | 0.9553 | 0.10810006 | 0.26019403 |
| Docosahexaenoic acid | 1.0563 | 1.2111 | 0.65170561 | 0.11562245 |
| (C22:cis[4,7,10,13,16,19]6) | | | | |
| myo-Inositol. lipid fraction | 1.031 | 1.1088 | 0.64375586 | 0.11852959 |
| 3,4-Dihydroxyphenylacetic | 1.0732 | 1.0153 | 0.11964385 | 0.73413494 |
| acid (DOPAC) | | | | |
| 4-Hydroxy-3- | 1.0271 | 0.9746 | 0.12610775 | 0.13529631 |
| methoxyphenylglycol | | | | |
| (HMPG) | | | | |
| gamma-Tocopherol | 0.9701 | 1.1195 | 0.68584079 | 0.1336499 |
| 5-Oxoproline | 0.9986 | 0.9463 | 0.97039848 | 0.13967798 |
| Phosphatidylcholine | 1.0055 | 1.0147 | 0.57978856 | 0.1411089 |
| (C16:0,C22:6) | | | | |
| 3-Hydroxybutyrate | 0.9659 | 1.0308 | 0.15035405 | 0.20860913 |
| 9-Hydroxyoctadecadienoic | 1.0125 | 1.0511 | 0.73197312 | 0.15196018 |
| acid (9-HODE) | | | | |
| (C18:trans[10]cis[12]2) | | | | |
| Allantoin | 0.8448 | 0.9405 | 0.15363982 | 0.60282307 |
| Cholesterylester C20:4 | 1.0626 | 1.083 | 0.27887799 | 0.15585149 |
| 3-Methoxytyrosine | 1.0612 | 1.0176 | 0.15809936 | 0.6734133 |
| 4-Hydroxy-3- | 1.0957 | 0.9915 | 0.16995783 | 0.89673187 |
| methoxymandelic acid | | | | |
| Docosapentaenoic acid | 0.9531 | 1.1762 | 0.6799843 | 0.17026722 |
| (C22:cis[4,7,10,13,16]5) | | | | |
| Cysteine | 1.1243 | 1.0049 | 0.17045624 | 0.95450467 |
| Ketoleucine | 0.9457 | 1.0215 | 0.17101675 | 0.60133095 |
| Glucose, lipid fraction | 0.9643 | 0.811 | 0.81224933 | 0.17258529 |
| trans-4-Hydroxyproline | 1.0076 | 1.0433 | 0.80738096 | 0.17429652 |
| Kynurenic acid | 0.843 | 0.6885 | 0.53361578 | 0.17473073 |
| Lysophosphatidylcholine | 0.9663 | 1.0486 | 0.32712459 | 0.17550525 |
| (C18:1) | | | | |
| Lysophosphatidylcholine | 0.9055 | 0.9254 | 0.18959921 | 0.30528294 |
| (C16:0) | | | | |
| Phosphatidylcholine | 0.973 | 1.0164 | 0.19077749 | 0.43417496 |
| (C16:0,C20:5) | | | | |
| Asparagine | 0.9754 | 0.9571 | 0.4576604 | 0.19218912 |
| Lactaldehyde | 0.7209 | 0.5925 | 1.91E−11 | 5.13E−23 |

TABLE 3′
Further biomarkers indicating quality issue
in plasma samples related to hemolysis

| | Hemolysis Grade 1 | Hemolysis Grade 1 |
|---|---|---|
| Biomarker (Metabolite) | Ratio relative to control | p-value |
| Threonic acid | 0.7313 | 5.6471E−08 |
| Aspartate | 0.6737 | 3.2136E−05 |
| Glucose | 0.9511 | 0.13980293 |
| Hypoxanthine | 0.8022 | 0.021891904 |
| Ribose | 0.8831 | 0.034991325 |
| 3-Phosphoglycerate (3- | 0.4448 | 1.50258E−06 |
| PGA) | | |

TABLE 3a
Preferred biomarkers indicating quality issue in plasma samples
related to hemolysis: Selection based on assayability.
Biomarker (Metabolite)

Taurine
Maltose
Glycerol-3-phosphate, polar fraction
Glutamate
Glycerate
Cystine
Cysteine
Asparagine

TABLE 3a′
Further preferred biomarkers indicating quality issue in plasma
samples related to hemolysis: Selection based on assayability.
Biomarker (Metabolite)

Threonic acid
Aspartate
Glucose
Hypoxanthine
Ribose
3-Phosphoglycerate (3-PGA)

TABLE 3c
Preferred biomarkers indicating quality issue in plasma samples
related to hemolysis: Selection based on method “GC-polar”.
Biomarker (Metabolite)

Taurine
Pyrophosphate (PPi)
Hypotaurine
Maltose
3-Hydroxyindole
Maltotriose
Glycerol-3-phosphate, polar fraction
Glutamate
myo-Inositol
Glycerol, polar fraction
Indole-3-acetic acid
Sulfate
Fumarate
beta-Alanine
Uric acid
Fructosamine
Glycolate
Sarcosine
1,5-Anhydrosorbitol
Alanine
Malate
Phosphate (inorganic and from organic phosphates)
2-Hydroxybutyrate
Glycerate
Cystine
Pseudouridine
Threitol
Isocitrate
5-Oxoproline
3-Hydroxybutyrate
Cysteine
trans-4-Hydroxyproline
Asparagine

TABLE 3c′
Further preferred biomarkers indicating quality issue in plasma samples
related to hemolysis: Selection based on method “GC-polar”.
Biomarker (Metabolite)

Threonic acid
Aspartate
Glucose
Hypoxanthine
Ribose
3-Phosphoglycerate (3-PGA)

TABLE 4
List of identified biomarkers indicating quality
issue in plasma samples related to microclotting

| | Microclotting | Microclotting |
|---|---|---|
| Biomarker (Metabolite) | Ratio relative to control | p-value |
| Sphingosine (d16:1) | 0.7388 | 0.00000056 |
| Sphingosine (d18:1) | 0.6147 | 0.00000124 |
| Taurine | 0.6963 | 0.00000278 |
| Hypotaurine | 0.7195 | 0.0000142 |
| Sphingadienine (d18:2) | 0.6975 | 0.000025 |
| Sphinganine (d18:0) | 0.7348 | 0.0000819 |
| Pyrophosphate (PPi) | 0.5964 | 0.00013249 |
| Sphingosine-1-phosphate | 0.8504 | 0.00021234 |
| (d18:1) | | |
| Sphinganine-1-phosphate | 0.7809 | 0.00039464 |
| (d18:0) | | |
| O-Phosphoethanolamine | 0.5917 | 0.00043076 |
| Leucine | 0.952 | 0.00064465 |
| Tetradecanol | 0.7024 | 0.00084462 |
| Alanine | 0.9556 | 0.00165354 |
| Valine | 0.9586 | 0.0020399 |
| myo-Inositol | 0.9281 | 0.00244972 |
| Glycerol, polar fraction | 0.8588 | 0.00419211 |
| 1.5-Anhydrosorbitol | 0.9404 | 0.0047325 |
| Lysine | 0.935 | 0.00500755 |
| Serine | 0.9553 | 0.00501809 |
| Kynurenic acid | 0.4618 | 0.00535807 |
| Proline | 0.9621 | 0.00563453 |
| Ornithine | 0.9427 | 0.00627566 |
| Glycine | 0.9647 | 0.00756625 |
| Sphingosine-1-phosphate | 0.9227 | 0.00868807 |
| (d17:1) | | |
| Cystine | 0.7395 | 0.01118095 |
| 9-Hydroxyoctadecadienoic | 1.0869 | 0.01722053 |
| acid (9-HODE) | | |
| (C18:trans[10]cis[12]2) | | |
| 8- | 0.8905 | 0.01722752 |
| Hydroxyeicosatetraenoic | | |
| acid | | |
| (C20:trans[5]cis[9,11,14]4) | | |
| (8-HETE) | | |
| Maltose | 0.6698 | 0.01774659 |
| Glutamine | 0.8273 | 0.01897131 |
| Erythrol | 0.922 | 0.02057815 |
| erythro- | 1.1902 | 0.02260725 |
| Dihydrosphingosine | | |
| (d18:0) | | |
| Tyrosine | 0.9562 | 0.02758202 |
| Pantothenic acid | 1.1862 | 0.03167457 |
| Histidine | 0.933 | 0.03517983 |
| Eicosanoic acid (C20:0) | 1.109 | 0.03856298 |
| Phosphatidylcholine | 1.0207 | 0.03894939 |
| (C16:0,C22:6) | | |
| Phenylalanine | 0.9629 | 0.0391365 |
| Serotonin (5-HT) | 0.6085 | 0.04015896 |
| Linolenic acid | 1.2093 | 0.04091573 |
| (C18:cis[9,12,15]3) | | |
| erythro-Sphingosine-1- | 1.1829 | 0.0418364 |
| phosphate (d18:1) | | |
| erythro-Sphingosine | 1.1242 | 0.0446753 |
| (d18:1) | | |
| Palmitic acid (C16:0) | 1.1872 | 0.04928017 |
| Isoleucine | 0.9629 | 0.04963676 |
| Linoleic acid | 1.1187 | 0.05166555 |
| (C18:cis[9.12]2) | | |
| Stearic acid (C18:0) | 1.1279 | 0.05459397 |
| Oleic acid (C18:cis[9]1) | 1.1505 | 0.05633266 |
| Lignoceric acid (C24:0) | 1.1226 | 0.05658935 |
| Cresol sulfate | 0.6572 | 0.05905597 |
| Taurochenodeoxycholic | 0.7842 | 0.06165658 |
| acid | | |
| Noradrenaline (Norepi- | 0.9296 | 0.06274978 |
| nephrine) | | |
| Nervonic acid | 1.1275 | 0.06313932 |
| (C24:cis[15]1) | | |
| Threonine | 0.9723 | 0.06325183 |
| Sphingomyelin | 1.0667 | 0.06408695 |
| (d18:2,C16:0) | | |
| Cholesta-2,4-dien | 1.1408 | 0.06875023 |
| Phosphatidylcholine | 1.0283 | 0.07727111 |
| (C18:0,C22:6) | | |
| Cholesterylester C18:1 | 1.0832 | 0.0777002 |
| Normetanephrine | 1.4479 | 0.07821874 |
| Dopamine | 0.8949 | 0.08117217 |
| Sphingadienine-1- | 0.9543 | 0.08192251 |
| phosphate (d18:2) | | |
| Fumarate | 1.0512 | 0.08314779 |
| Myristic acid (C14:0) | 1.2645 | 0.08468858 |
| 2-Hydroxybutyrate | 0.9706 | 0.08564891 |
| Ceramide (d18:1,C24:0) | 1.0735 | 0.08780931 |
| Nicotinamide | 0.8372 | 0.089989 |
| Hippuric acid | 0.7252 | 0.09246467 |
| Allantoin | 0.8195 | 0.09260986 |
| Glycerol phosphate, lipid | 1.1553 | 0.09644572 |
| fraction | | |
| Fructosamine | 0.7552 | 0.09695137 |
| Glycerol, lipid fraction | 1.2462 | 0.09759671 |
| 8,9- | 0.9166 | 0.10314504 |
| Dihydroxyeicosatrienoic | | |
| acid (C20:cis[5,11,14]3) | | |
| Asparagine | 0.9466 | 0.10325858 |
| Urea | 1.1031 | 0.10614621 |
| Glycerol-3-phosphate, | 0.8635 | 0.10719199 |
| polar fraction | | |
| Erythronic acid | 0.9384 | 0.11049428 |
| Tricosanoic acid (C23:0) | 1.1099 | 0.11396066 |
| Lactaldehyde | 0.9305 | 0.11485541 |
| 4-Hydroxy-3- | 0.9728 | 0.11575759 |
| methoxyphenylglycol | | |
| (HMPG) | | |
| Phosphate (inorganic and | 0.9519 | 0.1198119 |
| from organic phosphates) | | |
| Glucose, lipid fraction | 0.7883 | 0.12156488 |
| threo-Sphingosine (d18:1) | 1.0814 | 0.13031816 |
| Heptadecanoic acid | 1.098 | 0.13165303 |
| (C17:0) | | |
| 11- | 0.9183 | 0.13569715 |
| Hydroxyeicosatetraenoic | | |
| acid (C20:cis[5,8,12,14]4) | | |
| Choline plasmalogen | 0.9709 | 0.13973592 |
| (C18,C20:4) | | |
| Behenic acid (C22:0) | 1.0767 | 0.14079655 |
| alpha-Ketoglutarate | 0.8867 | 0.14845843 |
| 13- | 0.9631 | 0.14885392 |
| Hydroxyoctadecadienoic | | |
| acid (13-HODE) | | |
| (C18:cis[9]trans[11]2) | | |
| Phosphatidylcholine | 0.9898 | 0.15286642 |
| (C18:1,C18:2) | | |
| Glycolate | 1.1301 | 0.15389111 |
| Phosphatidylcholine | 0.9879 | 0.15432369 |
| (C18:0,C18:2) | | |
| Cortisol | 1.066 | 0.16441871 |
| 14,15- | 0.9323 | 0.16630566 |
| Dihydroxyeicosatrienoic | | |
| acid (C20:cis[5.8.11]3) | | |
| Indole-3-propionic acid | 0.6948 | 0.17404551 |
| Lysophosphatidylcholine | 1.0481 | 0.18012663 |
| (C18:1) | | |
| Isopalmitic acid (C16:0) | 1.1273 | 0.18099216 |
| 3-Indoxylsulfate | 1.2969 | 0.18733596 |
| Docosapentaenoic acid | 1.1129 | 0.19341371 |
| (C22:cis[7,10,13,16,19]5) | | |
| Sulfate | 1.1182 | 0.19688567 |
| Maltotriose | 0.6839 | 0.19842592 |

TABLE 4a
Preferred biomarkers indicating quality issue in plasma samples
related to microclotting: Selection based on assayability.
Biomarker (Metabolite)

Taurine
Ornithine
Cystine
Maltose
Glutamine
Asparagine
Glycerol-3-phosphate, polar fraction

TABLE 4b
Preferred biomarkers indicating quality issue in plasma samples
related to microclotting: Selection based on performance.
Biomarker (Metabolite)

Sphingosine (d16:1)
Sphingosine (d18:1)
Taurine
Hypotaurine
Sphingadienine (d18:2)
Sphinganine (d18:0)
Pyrophosphate (PPi)
Sphingosine-1-phosphate (d18:1)
Sphinganine-1-phosphate (d18:0)

TABLE 4c
Preferred biomarkers indicating quality issue in plasma samples
related to microclotting: Selection based on method “GC-polar”.
Biomarker (Metabolite)

Taurine
Hypotaurine
Pyrophosphate (PPi)
Leucine
Alanine
Valine
myo-Inositol
Glycerol, polar fraction
1,5-Anhydrosorbitol
Lysine
Serine
Proline
Ornithine
Glycine
Cystine
Maltose
Glutamine
Erythrol
Tyrosine
Histidine
Phenylalanine
Isoleucine
Threonine
Fumarate
2-Hydroxybutyrate
Fructosamine
Asparagine
Urea
Glycerol-3-phosphate, polar fraction
Erythronic acid
Phosphate (inorganic and from organic phosphates)
alpha-Ketoglutarate
Glycolate
Sulfate
Maltotriose

TABLE 5
List of identified biomarkers indicating quality issue in plasma samples related to contamination
with white blood cells.

| | contamination | contamination | | |
|---|---|---|---|---|
| | with blood cells | with blood cells | contamination | contamination |
| | grade 1 | grade 2 | with blood cells | with blood |
| | Ratio relative to | Ratio relative | grade 1 | cells grade 2 |
| Biomarker (Metabolite) | control | to control | p-value | p-value |
| Octadecanoylcarnitine | 1.0538 | 1.1317 | 0.15792455 | 0.0010021 |
| Eicosanoic acid (C20:0) | 1.05 | 1.1599 | 0.3267483 | 0.00321903 |
| Myristic acid (C14:0) | 1.3486 | 1.4972 | 0.02842173 | 0.00327954 |
| Glycerol-3-phosphate, polar | 1.086 | 1.2922 | 0.36482203 | 0.00345162 |
| fraction | | | | |
| Isopalmitic acid (C16:0) | 1.1113 | 1.2987 | 0.23848212 | 0.00385916 |
| myo-Inositol | 1.0196 | 1.0722 | 0.42391947 | 0.00457177 |
| scyllo-Inositol | 1.0465 | 1.1182 | 0.24871704 | 0.00503791 |
| Glycerol, polar fraction | 0.8624 | 0.9042 | 0.00531479 | 0.05631957 |
| Kynurenic acid | 0.465 | 0.6983 | 0.00576961 | 0.19156879 |
| Phosphatidylcholine | 0.9812 | 0.9907 | 0.00905959 | 0.19349866 |
| (C18:1,C18:2) | | | | |
| Stearic acid (C18:0) | 1.0796 | 1.1736 | 0.21987899 | 0.01086936 |
| Glycerol. lipid fraction | 1.4043 | 1.3607 | 0.01104028 | 0.02094199 |
| Palmitic acid (C16:0) | 1.1607 | 1.2424 | 0.08716715 | 0.01317795 |
| Tetradecanol | 0.7788 | 0.8117 | 0.0172088 | 0.04621911 |
| TAG (C18:1,C18:2,C18:3) | 1.1062 | 1.1363 | 0.05938846 | 0.01740461 |
| Glucose, lipid fraction | 0.6927 | 0.817 | 0.01741063 | 0.18804682 |
| beta-Carotene | 1.1207 | 1.1337 | 0.03059625 | 0.01747408 |
| Taurine | 1.0446 | 1.1952 | 0.5591166 | 0.01796886 |
| 15-Hydroxyeicosatetraenoic | 1.0075 | 1.1434 | 0.89212521 | 0.01798054 |
| acid (C20:cis[5,8,11,13]4) | | | | |
| Phosphatidylcholine | 1.0103 | 1.0237 | 0.29924859 | 0.01824443 |
| (C16:0,C22:6) | | | | |
| 14-Methylhexadecanoic acid | 1.1619 | 1.2471 | 0.11511791 | 0.0209762 |
| erythro-Dihydrosphingosine | 1.0591 | 1.1917 | 0.44917627 | 0.02164197 |
| (d18:0) | | | | |
| Fumarate | 1.0669 | 1.0299 | 0.02490096 | 0.30439824 |
| Phosphatidylcholine | 1.0361 | 1.0338 | 0.02548469 | 0.03599054 |
| (C18:0,C22:6) | | | | |
| 12-Hydroxyeicosatetraenoic | 1.2529 | 1.4951 | 0.20923695 | 0.02795269 |
| acid (C20:cis[5,8,10,14]4) | | | | |
| Ceramide (d18:1,C24:0) | 1.0663 | 1.0953 | 0.12219628 | 0.02893994 |
| 4-Hydroxysphinganine (t18:0, | 0.8726 | 1.0828 | 0.03204245 | 0.20874813 |
| Phytosphingosine), total | | | | |
| Sphingosine-1-phosphate | 0.9518 | 0.9934 | 0.03318565 | 0.77742428 |
| (d16:1) | | | | |
| Noradrenaline (Norepinephrine) | 1.0174 | 1.0872 | 0.65922517 | 0.03319438 |
| Cholesterylester C18:1 | 1.1002 | 1.0996 | 0.03551268 | 0.03643532 |
| Linolenic acid | 1.1966 | 1.2109 | 0.05338709 | 0.03957424 |
| (C18:cis[9,12,15]3) | | | | |
| Hypoxanthine | 1.0804 | 1.2089 | 0.4025602 | 0.04101644 |
| alpha-Ketoglutarate | 0.9111 | 0.842 | 0.26275036 | 0.04231871 |
| Sphingomyelin (d18:1,C23:0) | 1.0625 | 1.0521 | 0.04732452 | 0.09571044 |
| Hexadecanoylcarnitine | 1.033 | 1.0838 | 0.42352152 | 0.0480908 |
| Lysophosphatidylcholine | 0.862 | 0.9528 | 0.05036623 | 0.52180346 |
| (C16:0) | | | | |
| Hydroxyhexadecenoylcarnitine | 0.9915 | 1.157 | 0.90832309 | 0.05047124 |
| Lignoceric acid (C24:0) | 1.0263 | 1.1259 | 0.66666178 | 0.05061619 |
| Tricosanoic acid (C23:0) | 1.0372 | 1.1369 | 0.57872264 | 0.05225991 |
| Phosphate, lipid fraction | 1.0434 | 1.1219 | 0.49211379 | 0.05257546 |
| Coenzyme Q10 | 1.0562 | 1.0798 | 0.16736997 | 0.05307157 |
| Indole-3-lactic acid | 1.0671 | 1.0165 | 0.05391152 | 0.62585796 |
| Nicotinamide | 1.0995 | 1.2239 | 0.36418519 | 0.05423505 |
| gamma-Linolenic acid | 1.0702 | 1.188 | 0.45429192 | 0.05863261 |
| (C18:cis[6,9,12]3) | | | | |
| Ceramide (d18:1,C24:1) | 1.0451 | 1.0811 | 0.28428485 | 0.05900713 |
| 1-Hydroxy-2-amino-(cis,trans)- | 0.9956 | 1.1553 | 0.95364978 | 0.05921965 |
| 3,5-octadecadiene (from | | | | |
| sphingolipids) | | | | |
| trans-4-Hydroxyproline | 1.0608 | 1.0212 | 0.05939296 | 0.5006412 |
| Phosphatidylcholine | 0.996 | 0.984 | 0.6395939 | 0.06106037 |
| (C18:0,C18:2) | | | | |
| Linoleic acid (C18:cis[9,12]2) | 1.0555 | 1.114 | 0.3470017 | 0.06108522 |
| Citrulline | 0.8082 | 1.0075 | 0.06220195 | 0.94743022 |
| Glucose-1-phosphate | 1.082 | 1.0985 | 0.12040529 | 0.06432961 |
| Oxalate | 0.9408 | 0.8781 | 0.38574462 | 0.06552835 |
| erythro-Sphingosine (d18:1) | 1.0172 | 1.1128 | 0.76889784 | 0.0664724 |
| erythro-Sphingosine-1- | 1.0108 | 1.162 | 0.89567734 | 0.06857326 |
| phosphate (d18:1) | | | | |
| beta-Alanine | 1.0514 | 1.0827 | 0.25917359 | 0.07430839 |
| Galactose, lipid fraction | 0.965 | 1.0915 | 0.47095031 | 0.07766798 |
| Cholesterylester C20:4 | 1.0418 | 1.104 | 0.465448 | 0.07881676 |
| Erythrol | 0.9404 | 0.9906 | 0.07894244 | 0.78519053 |
| Sphingosine-1-phosphate | 0.948 | 1.0086 | 0.07995434 | 0.78127354 |
| (d17:1) | | | | |
| Urea | 1.0125 | 1.1122 | 0.83696726 | 0.0800395 |
| Cholesta-2,4-dien | 1.0055 | 1.1348 | 0.93909925 | 0.08053479 |
| Phosphatidylcholine | 1.0326 | 1.0192 | 0.0809958 | 0.30023543 |
| (C16:1,C18:2) | | | | |
| Oleic acid (C18:cis[9]1) | 1.1331 | 1.1341 | 0.08872985 | 0.0864597 |
| Phosphatidylcholine | 1.0166 | 1.0323 | 0.372939 | 0.08666552 |
| (C16:0,C16:0) | | | | |
| Glycerol phosphate, lipid fraction | 1.0307 | 1.158 | 0.72683321 | 0.09112788 |
| Behenic acid (C22:0) | 0.9936 | 1.0877 | 0.89746608 | 0.09411067 |
| Pseudouridine | 1.043 | 1.0569 | 0.20784036 | 0.09805914 |
| Heptadecanoic acid (C17:0) | 1.0605 | 1.108 | 0.34274251 | 0.09837302 |
| Phosphate (inorganic and | 0.958 | 0.9493 | 0.17618677 | 0.10083665 |
| from organic phosphates) | | | | |
| erythro-Dihydrosphingosine | 0.9838 | 1.1788 | 0.87129422 | 0.10419427 |
| (d16:0) | | | | |
| Histamine | 0.9888 | 1.3822 | 0.95506696 | 0.10769205 |
| Cortisol | 1.0765 | 1.059 | 0.10877382 | 0.21171679 |
| 5-Oxoproline | 0.9655 | 0.9418 | 0.34677778 | 0.10881936 |
| Docosapentaenoic acid | 1.0425 | 1.1408 | 0.61239363 | 0.10985169 |
| (C22:cis[7,10,13,16,19]5) | | | | |
| Maltose | 1.1218 | 1.3078 | 0.49314602 | 0.11068058 |
| Phosphatidylcholine | 1.0339 | 1.026 | 0.11118295 | 0.21923346 |
| (C16:0,C20:5) | | | | |
| Coenzyme Q9 | 1.0983 | 1.1071 | 0.14231115 | 0.11153678 |
| Phosphatidylcholine | 1.007 | 1.0179 | 0.53617563 | 0.11431758 |
| (C18:0,C18:1) | | | | |
| Adrenaline (Epinephrine) | 1.0316 | 1.0747 | 0.50829486 | 0.13200942 |
| Sphingomyelin (d18:1,C24:0) | 1.0196 | 1.0283 | 0.30567931 | 0.14035551 |
| Serine, lipid fraction | 0.8982 | 1.2487 | 0.47997343 | 0.14490568 |
| Lysophosphatidylcholine | 1.0756 | 1.0088 | 0.14541851 | 0.86072139 |
| (C18:0) | | | | |
| 4-Hydroxy-3- | 0.9754 | 0.991 | 0.14816483 | 0.59853269 |
| methoxyphenylglycol (HMPG) | | | | |
| Creatine | 1.0695 | 1.0544 | 0.14859097 | 0.25433904 |
| Fructosamine | 1.1318 | 1.2758 | 0.45021027 | 0.14945026 |
| Serotonin (5-HT) | 1.0861 | 1.4155 | 0.72721986 | 0.14979383 |
| Phosphatidylcholine | 0.997 | 0.9898 | 0.68078744 | 0.15547554 |
| (C16:0,C20:4) | | | | |
| Lysophosphatidylcholine | 1.0572 | 1.0379 | 0.15565027 | 0.34193337 |
| (C17:0) | | | | |
| Hypotaurine | 1.0568 | 1.1102 | 0.45937607 | 0.15668129 |
| Sphingomyelin (d18:2,C18:0) | 1.0298 | 1.0275 | 0.15848535 | 0.19227455 |
| dihomo-gamma-Linolenic acid | 1.0211 | 1.104 | 0.76647777 | 0.16020882 |
| (C20:cis[8,11,14]3) | | | | |
| Normetanephrine | 1.2169 | 1.358 | 0.37230656 | 0.16511127 |
| Uric acid | 1.0168 | 1.0371 | 0.52422393 | 0.16570125 |
| Palmitoleic acid (C16:cis[9]1) | 1.1471 | 1.1587 | 0.20444291 | 0.17321061 |
| Glutamate | 0.9791 | 1.1149 | 0.79269688 | 0.17719315 |
| TAG (C16:0,C18:1,C18:3) | 1.0871 | 1.0643 | 0.17839248 | 0.31459893 |
| threo-Sphingosine (d18:1) | 0.9892 | 1.0717 | 0.83283371 | 0.18024004 |
| Lysophosphatidylcholine | 1.0229 | 1.0481 | 0.51671833 | 0.18027525 |
| (C18:1) | | | | |
| 3.4-Dihydroxyphenylacetic | 0.9747 | 1.062 | 0.57153816 | 0.18439449 |
| acid (DOPAC) | | | | |
| 11-Hydroxyeicosatetraenoic | 1.0221 | 1.0797 | 0.70036603 | 0.18523073 |
| acid (C20:cis[5,8,12,14]4) | | | | |
| Pantothenic acid | 0.9923 | 1.1103 | 0.92160171 | 0.18612223 |
| 3-Hydroxybutyrate | 1.0323 | 1.0009 | 0.18678051 | 0.96959232 |
| Glycerate | 1.0755 | 1.0841 | 0.2347587 | 0.18730616 |

TABLE 5′
Further biomarker indicating quality issue in plasma
samples related to contamination with white blood cells.

| | contamination with | contamination with |
|---|---|---|
| | blood cells grade 2 | blood cells grade 2 |
| Biomarker (Metabolite) | Ratio relative to control | p-value |
| Threonic acid | 0.776 | 7.43E−06 |

Threonic acid is also a further preferred biomarker indicating quality issue in plasma samples related to contamination with blood cells in selection based on assayability, and/or based on method “GC-polar”.

TABLE 5a
Preferred biomarkers indicating quality issue in plasma samples related
to contamination with white blood cells: Selection based on assayability.
Biomarker (Metabolite)

Glycerol-3-phosphate, polar fraction
Taurine
Hypoxanthine
Maltose
Glutamate
Glycerate

TABLE 5b
Preferred biomarkers indicating quality issue in plasma samples related
to contamination with white blood cells: Selection based on performance.
Biomarker (Metabolite)

Octadecanoylcarnitine
Eicosanoic acid (C20:0)
Myristic acid (C14:0)
Glycerol-3-phosphate, polar fraction
Isopalmitic acid (C16:0)
myo-Inositol
scyllo-Inositol

TABLE 5c
Preferred biomarkers indicating quality issue in
plasma samples related to contamination with white
blood cells: Selection based on method “GC-polar”.
Biomarker (Metabolite)

Glycerol-3-phosphate, polar fraction
myo-Inositol
scyllo-Inositol
Glycerol, polar fraction
Taurine
Fumarate
Hypoxanthine
alpha-Ketoglutarate
trans-4-Hydroxyproline
Glucose-1-phosphate
Oxalate
beta-Alanine
Erythrol
Pseudouridine
Phosphate (inorganic and from organic phosphates)
5-Oxoproline
Maltose
Fructosamine
Hypotaurine
Uric acid
Glutamate
3-Hydroxybutyrate
Glycerate

TABLE 6
List of identified biomarkers indicating quality issue in plasma samples related to storage.

| | Samples stored at −20° C. relative to samples stored at −196° C. | | | |
|---|---|---|---|---|
| | 181 days | 365 days | 181 days | 365 days |
| Biomarker (Metabolite) | Ratio | Ratio | p-value | p-value |
| Glutamate | 2.6368 | 4.9119 | 0.00033723 | 0.00017925 |
| Glutamine | 0.656 | 0.6304 | 0.00291477 | 0.00348084 |
| Aspartate | 2.0239 | 6.8136 | 0.008366 | 0.00197954 |
| Asparagine | 0.8506 | 0.8243 | 0.08098985 | 0.01849786 |
| Phosphatidylcholine hydroper- | 1.4599 | 3.1993 | 0.08455524 | 0.00010079 |
| oxide (C16:0, C18:2-OOH) | | | | |
| Phosphatidylcholine hydroper- | 3.4881 | 16.1379 | 0.0000657 | 0.00000104 |
| oxide (C16:0, C18:1-OOH) | | | | |
| Phosphatidylcholine hydroper- | 1.6789 | 2.5742 | 0.00683629 | 0.00013176 |
| oxide (C18:0, C18:2-OOH) | | | | |
| Triacylgyceride hydroperoxide | 4.4365 | 35.6414 | 0.06669905 | 0.00000042 |
| (C16:0, C18:1, C18:3-OOH) | | | | |
| Triacylgyceride hydroperoxide | 4.4365 | 35.6414 | 0.06669905 | 0.00000042 |
| (C16:0, C18:2, C18:2-OOH) | | | | |
| Triacylgyceride hydroperoxide | 2.3951 | 21.9135 | 0.00247007 | 0.00000004 |
| (C16:0, C18:1, C18:2-OOH) | | | | |
| Triacylgyceride hydroperoxide | | 74.6446 | | 2.86E−09 |
| (C18:1, 18:2, C18:2-OOH) | | | | |
| Triacylgyceride hydroperoxide | | 74.6446 | | 2.86E−09 |
| (C16:0, C18:1, C20:4-OOH) | | | | |
| Triacylgyceride hydroperoxide | | 74.6446 | | 2.86E−09 |
| (C18:1, C18:1, C18:3-OOH) | | | | |
| Cholesterylester hydroperoxide | 17.6715 | 38.5989 | 0.0000132 | 4.62E−07 |
| (C18:2-9-OOH) | | | | |
| Cholesterylester hydroperoxide | 17.6715 | 38.5989 | 0.0000132 | 4.62E−07 |
| (C18:2-13-OOH) | | | | |
| Cholesterylester hydroperoxide | 17.6715 | 38.5989 | 0.0000132 | 4.62E−07 |
| (C20:4-OOH) | | | | |
| Cholesterylester hydroperoxide | 2.2942 | 8.6105 | 0.009491 | 0.0000101 |
| (C18:2-9-OOH) | | | | |
| Cholesterylester hydroperoxide | 2.2942 | 8.6105 | 0.009491 | 0.0000101 |
| (C18:2-13-OOH) | | | | |
| Prostaglandin E2 | 10.9228 | 127.7318 | 0.00696699 | 1.46E−07 |
| 3,4-Dihydroxyphenylalanine | 0.1155 | 0.0743 | 1.07E−07 | 2.44E−07 |
| (DOPA) | | | | |
| 3,4-Dihydroxyphenylglycol | 0.2073 | 0.0534 | 1.48E−08 | 2.97E−07 |
| (DOPEG) | | | | |
| Cysteine | 0.6352 | 0.48 | 0.01568556 | 3.78E−07 |
| Cystine | 0.3273 | 0.272 | 0.01170524 | 6.79E−07 |
| Noradrenaline (Norepinephrine) | 0.2864 | 0.0491 | 0.0000203 | 0.000002 |
| Pyruvate | 0.7416 | 0.3825 | 0.00408159 | 0.00000288 |
| 3,4-Dihydroxyphenylacetic acid | 0.0172 | 0.0057 | 4.77E−10 | 0.00000372 |
| (DOPAC) | | | | |
| Glycerate | 2.1608 | 3.6579 | 0.00073095 | 0.000005 |
| 13,14-Dihydro-15- | 1.4026 | 3.7203 | 0.77268436 | 0.0000443 |
| ketoprostaglandin D2 | | | | |
| Adrenaline (Epinephrine) | 1.0395 | 0.1335 | 0.1334819 | 0.0000651 |
| delta-12-Prostaglandin J2 | 5.2127 | 124.0942 | 0.28477496 | 0.0000892 |
| 4-Hydroxyphenylpyruvate | 0.6479 | 0.3398 | 0.01209475 | 0.00036356 |
| Prostaglandin D2 | 39.7057 | 775.1853 | 0.00085332 | 0.00042654 |
| Lipoxin A4 | 85.1016 | 125.1515 | 0.00670764 | 0.00079235 |
| 8,9-Epoxyeicosatrienoic acid | 2.0874 | 3.4474 | 0.02347563 | 0.00116761 |
| (C20:cis[5,11,14]3) | | | | |
| Prostaglandin F2 alpha | 2.7009 | 6.8075 | 0.03476308 | 0.00123974 |
| beta-Carotene | 0.8524 | 0.62 | 0.04723278 | 0.00135881 |
| 5-Oxoproline | 1.0552 | 1.3387 | 0.67135719 | 0.00192537 |
| Coenzyme Q10 | 0.7681 | 0.6624 | 0.07951077 | 0.00374187 |
| Prostaglandin J2 | 16.6378 | 160.2748 | 0.09776237 | 0.00598662 |
| Diacylglceride (C18:1, C18:2) | 0.9258 | 0.8026 | 0.15669889 | 0.00615809 |
| 6-Oxoprostaglandin F1 alpha | 1.171 | 3.1176 | 0.67692366 | 0.00665208 |
| delta-12-Prostaglandin D2 | 612.6522 | 311.398 | 0.00439507 | 0.00728037 |
| Thromboxane B2 | 0.2906 | 0.5613 | 0.14071353 | 0.00793485 |
| 12-Hydroxyeicosatetraenoic | 1.886 | 19.3798 | 0.11477199 | 0.0088226 |
| acid (C20:cis[5,8,10,14]4) | | | | |
| Arachidonic acid | 0.8719 | 0.8851 | 0.00840523 | 0.01033471 |
| (C20:cis[5,8,11,14]4) | | | | |
| 5-Hydroxyeicosatetraenoic acid | 3.307 | 26.5618 | 0.12306914 | 0.01189927 |
| (C20:trans[6]cis[8,11,14]4) | | | | |
| Docosahexaenoic acid | 0.8699 | 0.8436 | 0.0030645 | 0.01200993 |
| (C22:cis[4,7,10,13,16,19]6) | | | | |
| Glycerol, polar fraction | 0.9656 | 1.3693 | 0.84358345 | 0.01379894 |
| 15-Deoxy-delta(12,14)- | 7.6596 | 10.224 | 0.00403572 | 0.01545605 |
| prostaglandin J2 | | | | |
| Leukotriene B4 | 6.6204 | 60.1663 | 0.01386475 | 0.01825921 |
| 17,18-Epoxyarachidonic acid | 1.0416 | 8.1826 | 0.61483042 | 0.01827305 |
| (C20:cis[5,8,11,14]4) | | | | |
| 8-Hydroxyeicosatetraenoic acid | 1.8747 | 24.9631 | 0.20380341 | 0.01951045 |
| (C20:trans[5]cis[9,11,14]4) | | | | |
| 9-Hydroxyoctadecadienoic acid | 1.3359 | 5.8508 | 0.42403759 | 0.02241876 |
| (9-HODE) | | | | |
| (C18:trans[10]cis[12]2) | | | | |
| 15-Hydroxyeicosatetraenoic | 1.7003 | 22.11 | 0.247131 | 0.02367977 |
| acid (C20:cis[5,8,11,13]4) | | | | |
| Corticosterone | 0.9082 | 0.8102 | 0.35104355 | 0.02405416 |
| 14,15-Epoxyeicosatrienoic acid | 3.991 | 3.6061 | 0.26875533 | 0.02408394 |
| (C20:cis[5,8,11]3) | | | | |
| 13-Hydroxyoctadecadienoic | 1.2494 | 4.3411 | 0.48986081 | 0.02472036 |
| acid (13-HODE) | | | | |
| (C18:cis[9]trans[11]2) | | | | |
| 11-Hydroxyeicosatetraenoic | 1.5862 | 20.7365 | 0.1939883 | 0.02496287 |
| acid (C20:cis[5,8,12,14]4) | | | | |
| Cholesterol, total | 0.9485 | 0.9163 | 0.26956024 | 0.02684661 |
| Threonic acid | 0.5485 | 1.2215 | 0.00288993 | 0.02730538 |
| Triacylglyceride (C18:2, C18:3) | 0.9112 | 0.8154 | 0.52695373 | 0.02894779 |
| Canthaxanthin | 1.0326 | 0.7673 | 0.65218392 | 0.0349368 |
| Eicosapentaenoic acid | 0.8784 | 0.8641 | 0.01426751 | 0.04267106 |
| (C20:cis[5,8,11,14,17]5) | | | | |
| Cryptoxanthin | 0.9022 | 0.7149 | 0.23539907 | 0.04535175 |
| Cresol sulfate | 1.0766 | 0.4362 | 0.89476965 | 0.06057201 |
| 11,12-Epoxyeicosatrienoic acid | 0.8515 | 3.6099 | 0.72264104 | 0.06827723 |
| (C20:cis[5,8,14]3) | | | | |
| Citrulline | 0.9785 | 1.1009 | 0.65160998 | 0.07227598 |
| Phosphate (inorganic and from | 0.9262 | 1.2145 | 0.2791334 | 0.0730049 |
| organic phosphates) | | | | |
| gamma-Linolenic acid | 0.8138 | 0.8863 | 0.00173952 | 0.07656861 |
| (C18:cis[6,9,12]3) | | | | |
| Linoleic acid (C18:cis[9,12]2) | 0.8432 | 0.9496 | 0.00832098 | 0.11276713 |
| Glucose | 0.8651 | 0.8821 | 0.02802542 | 0.13018966 |
| Oleic acid (C18:cis[9]1) | 0.8791 | 0.9548 | 0.0384577 | 0.13232617 |
| dihomo-gamma-Linolenic acid | 0.9057 | 0.9438 | 0.03673706 | 0.18819684 |
| (C20:cis[8,11,14]3) | | | | |

TABLE 6a
Preferred biomarkers indicating quality issue in plasma
samples related to storage: Selection based on assayability.
Biomarker (Metabolite)

Glutamate
Glutamine
Aspartate
Asparagine
Cysteine
Cystine
Glycerate
Threonic acid
Glucose

TABLE 6b
Preferred biomarkers indicating quality issue in plasma
samples related to storage: Selection based on performance.
Biomarker (Metabolite)

Glutamate
Glutamine
Aspartate
Asparagine
Phosphatidylcholine hydroperoxide (C16:0, C18:2-OOH)
Phosphatidylcholine hydroperoxide (C16:0, C18:1-OOH)
Phosphatidylcholine hydroperoxide (C18:0, C18:2-OOH)
Triacylgyceride hydroperoxide (C16:0, C18:1, C18:3-OOH)
Triacylgyceride hydroperoxide (C16:0, C18:2, C18:2-OOH)
Triacylgyceride hydroperoxide (C16:0, C18:1, C18:2-OOH)
Triacylgyceride hydroperoxide (C18:1, 18:2, C18:2-OOH)
Triacylgyceride hydroperoxide (C16:0, C18:1, C20:4-OOH)
Triacylgyceride hydroperoxide (C18:1, C18:1, C18:3-OOH)
Cholesterylester hydroperoxide (C18:2-9-OOH)
Cholesterylester hydroperoxide (C18:2-13-OOH)
Cholesterylester hydroperoxide (C20:4-OOH)
Cholesterylester hydroperoxide (C18:2-9-OOH)
Cholesterylester hydroperoxide (C18:2-13-OOH)

TABLE 6c
Preferred biomarkers indicating quality issue in plasma samples
related to storage: Selection based on method “GC-polar”.
Biomarker (Metabolite)

Glutamate
Glutamine
Aspartate
Asparagine
Cysteine
Cystine
Pyruvate
Glycerate
5-Oxoproline
Glycerol, polar fraction
Threonic acid
Phosphate (inorganic and from organic phosphates)
Glucose

TABLE 7
List of identified biomarkers indicating quality
issue due to slow freezing of samples

| | Slow | |
|---|---|---|
| | freezing | |
| | Ratio | Slow |
| | relative | freezing |
| Biomarker (Metabolite) | to control | p-value |
| Tetradecanol | 0.7232 | 0.002140022 |
| Urea | 1.1689 | 0.010627 |
| Stearic acid (C18:0) | 1.1687 | 0.013118363 |
| Eicosanoic acid (C20:0) | 1.1322 | 0.013354788 |
| Erythrol | 0.9189 | 0.015905156 |
| erythro-Dihydrosphingosine (d18:0) | 1.2011 | 0.016502956 |
| Lysophosphatidylethanolamine (C22:5) | 1.1687 | 0.017126683 |
| Myristic acid (C14:0) | 1.3804 | 0.018294628 |
| Linoleic acid (C18:cis[9,12]2) | 1.1455 | 0.018787093 |
| Lignoceric acid (C24:0) | 1.1526 | 0.019598109 |
| Linolenic acid (C18:cis[9,12,15]3) | 1.2329 | 0.024482544 |
| Sphingomyelin (d18:2, C16:0) | 1.0812 | 0.025463963 |
| Glycerol, polar fraction | 0.8895 | 0.026854543 |
| Phosphate, lipid fraction | 1.1494 | 0.028126069 |
| Palmitic acid (C16:0) | 1.2108 | 0.028599728 |
| Phosphatidylcholine (C18:1, C18:2) | 0.9844 | 0.029842398 |
| Behenic acid (C22:0) | 1.1154 | 0.030111761 |
| Normetanephrine | 1.5659 | 0.030914681 |
| Glycerol, lipid fraction | 1.33 | 0.032321451 |
| TAG (C18:1, C18:2, C18:3) | 1.1204 | 0.033967485 |
| Oleoylcarnitine | 1.0784 | 0.045873499 |
| Tricosanoic acid (C23:0) | 1.1403 | 0.047055468 |
| Octadecanoylcarnitine | 1.0743 | 0.053980018 |
| 12-Hydroxyeicosatetraenoic acid | 1.4117 | 0.055602301 |
| (C20:cis[5,8,10,14]4) | | |
| Cystine | 0.7985 | 0.057477558 |
| Serine. lipid fraction | 1.3297 | 0.061960511 |
| myo-Inositol-2-phosphate, lipid fraction | 0.8093 | 0.074002009 |
| (myo-Inositolphospholipids) | | |
| 11,12-Dihydroxyeicosatrienoic acid | 0.9289 | 0.077575628 |
| (C20:cis[5,8,14]3) | | |
| alpha-Ketoglutarate | 0.8636 | 0.078577812 |
| Kynurenic acid | 0.6216 | 0.084333549 |
| Sphingosine-1-phosphate (d16:1) | 0.9612 | 0.087576006 |
| Asparagine | 0.9443 | 0.088710231 |
| gamma-Tocopherol | 0.8797 | 0.088828878 |
| Glutamate | 1.1448 | 0.093928971 |
| 3,4-Dihydroxyphenylalanine (DOPA) | 0.9602 | 0.097117107 |
| 3-Methoxytyrosine | 1.0707 | 0.100262462 |
| Cholesterol, free | 1.0389 | 0.101823511 |
| Oleic acid (C18:cis[9]1) | 1.1274 | 0.102262627 |
| Indole-3-acetic acid | 0.9432 | 0.103924616 |
| 1-Hydroxy-2-amino-(cis,trans)-3,5- | 1.1299 | 0.109874857 |
| octadecadiene (from sphingolipids) | | |
| Hexadecanoylcarnitine | 1.0671 | 0.109937475 |
| Indole-3-lactic acid | 1.0551 | 0.110939097 |
| erythro-Sphingosine-1-phosphate (d18:1) | 1.1397 | 0.112147051 |
| Methionine | 1.0293 | 0.112264262 |
| erythro-Dihydrosphingosine (d16:0) | 1.171 | 0.11894756 |
| Fumarate | 1.0448 | 0.127704433 |
| Eicosapentaenoic acid | 1.1927 | 0.131265492 |
| (C20:cis[5,8,11,14,17]5) | | |
| Glycerate | 1.096 | 0.13481564 |
| Sphingosine-1-phosphate (d17:1) | 0.9556 | 0.135505349 |
| Salicylic acid | 0.7635 | 0.142392241 |
| Tryptophan | 1.0369 | 0.150124043 |
| Isopalmitic acid (C16:0) | 1.1327 | 0.164361792 |
| trans-4-Hydroxyproline | 1.0442 | 0.165452043 |
| Phosphatidylcholine (C18:0, C20:4) | 1.0093 | 0.170866449 |
| Hypoxanthine | 1.1367 | 0.172377725 |
| Glucose-6-phosphate | 1.251 | 0.174943248 |
| gamma-Linolenic acid (C18:cis[6,9,12]3) | 1.1292 | 0.181226918 |
| Ceramide (d18:1, C24:1) | 1.0564 | 0.183183249 |
| Sphingomyelin (d18:1, C23:0) | 1.0413 | 0.184103295 |
| Glutamine | 0.8991 | 0.185532729 |
| Pantothenic acid | 1.1104 | 0.185753913 |
| Hippuric acid | 0.7794 | 0.191123874 |
| 14-Methylhexadecanoic acid | 1.1323 | 0.191485912 |
| Sphingomyelin (d18:2, C18:0) | 1.0275 | 0.19216552 |
| beta-Carotene | 1.0708 | 0.192624577 |
| erythro-Sphingosine (d18:1) | 1.0777 | 0.197612672 |
| Pyrophosphate (PPi) | 0.8431 | 0.198160999 |

TABLE 7a
Preferred biomarkers indicating quality issue due to slow
freezing of samples: Selection based on assayability.
Biomarker (Metabolite)

Cystine
Asparagine
Glutamate
Glycerate
Hypoxanthine
Glutamine

TABLE 7c
Preferred biomarkers indicating quality issue due to slow
freezing of samples: Selection based on method “GC-polar”.
Biomarker (Metabolite)

Erythrol
Glycerol, polar fraction
Cystine
alpha-Ketoglutarate
Asparagine
Glutamate
Indole-3-acetic acid
Methionine
Fumarate
Glycerate
Tryptophan
trans-4-Hydroxyproline
Hypoxanthine
Glutamine
Pyrophosphate (PPi)

TABLE 8
List of identified biomarkers indicating quality issue in
serum samples related to pro-longed coagulation of blood.

Effect of increased
coagulation period of
blood relative to direct
processing to serum

| Biomarker (Metabolite) | Ratio | p-value |
|---|---|---|
| Malate | 3.45 | 5.18E−53 |
| Glycerol-3-phosphate, polar fraction | 4.40 | 4.27E−50 |
| Pyruvate | 3.48 | 4.43E−43 |
| Arginine | 0.43 | 9.73E−43 |
| 5-Oxoproline | 1.59 | 2.25E−36 |
| Ornithine | 2.01 | 6.15E−36 |
| Mannose | 0.35 | 5.81E−35 |
| Glutamate | 3.92 | 1.31E−34 |
| Cysteine | 2.14 | 6.81E−33 |
| 8-Hydroxyeicosatetraenoic acid | 11.79 | 4.12E−31 |
| (C20:trans[5]cis[9,11,14]4) (8-HETE) | | |
| alpha-Ketoglutarate | 4.02 | 1.24E−30 |
| Aspartate | 2.55 | 1.08E−29 |
| Lysophosphatidylcholine (C18:0) | 1.61 | 1.25E−29 |
| 13-Hydroxyoctadecadienoic acid | 4.07 | 4.56E−29 |
| (13-HODE) (C18:cis[9]trans[11]2) | | |
| 15-Hydroxyeicosatetraenoic acid | 6.33 | 1.13E−28 |
| (C20:cis[5,8,11,13]4) | | |
| 12-Hydroxyeicosatetraenoic acid | 7.28 | 2.61E−28 |
| (C20:cis[5,8,10,14]4) | | |
| Serine | 1.47 | 2.56E−25 |
| Glucose-6-phosphate | 2.75 | 4.66E−25 |
| Phenylalanine | 1.47 | 6.22E−24 |
| 3,4-Dihydroxyphenylglycol | 0.50 | 2.07E−23 |
| (DOPEG) | | |
| Lysophosphatidylcholine (C17:0) | 1.56 | 4.25E−23 |
| 9-Hydroxyoctadecadienoic acid | 3.18 | 1.27E−22 |
| (9-HODE) (C18:trans[10]cis[12]2) | | |
| Phosphate (inorganic and from | 1.77 | 2.59E−20 |
| organic phosphates) | | |
| Glycerate | 1.66 | 3.63E−19 |
| Glycine | 1.52 | 4E−18 |
| 8,9-Dihydroxyeicosatrienoic acid | 1.99 | 5.67E−17 |
| (C20:cis[5,11,14]3) | | |
| Alanine | 1.42 | 1.94E−16 |
| Asparagine | 1.48 | 4.18E−16 |
| Taurine | 1.73 | 1.52E−15 |
| Lysine | 1.36 | 2.07E−14 |
| Prostaglandin F2 alpha | 3.48 | 6.67E−14 |
| Xanthine | 1.50 | 2.41E−13 |
| myo-Inositol | 1.34 | 4.54E−13 |
| Lysophosphatidylcholine (C16:0) | 1.23 | 5.36E−13 |
| Leucine | 1.35 | 1E−12 |
| 11-Hydroxyeicosatetraenoic acid | 4.07 | 2.6E−12 |
| (C20:cis[5,8,12,14]4) | | |
| Histidine | 1.26 | 1.53E−11 |
| Lysophosphatidylcholine (C18:1) | 1.18 | 2.97E−11 |
| Lysophosphatidylethanolamine (C22:5) | 1.19 | 3.62E−11 |
| Lysophosphatidylcholine (C20:4) | 1.26 | 1.8E−10 |
| Noradrenaline (Norepinephrine) | 0.59 | 4.81E−10 |
| Erythrol | 1.30 | 6.99E−10 |
| Cystine | 1.49 | 7.58E−10 |
| Mannosamine | 0.59 | 6.5E−09 |
| Threonic acid | 1.49 | 8.04E−09 |
| Glucosamine | 0.67 | 2.75E−08 |
| Maltose | 1.61 | 0.000000112 |
| Valine | 1.19 | 0.000000392 |
| 11,12-Dihydroxyeicosatrienoic | 1.46 | 0.000000738 |
| acid (C20:cis[5,8,14]3) | | |
| 5-Hydroxy-3-indoleacetic acid | 1.56 | 0.00000256 |
| (5-HIAA) | | |
| Ketoleucine | 1.27 | 0.0000027 |
| Isoleucine | 1.25 | 0.00000289 |
| 5-Hydroxyeicosatetraenoic acid | 2.06 | 0.00000321 |
| (C20:trans[6]cis[8,11,14]4) | | |
| (5-HETE) | | |
| Methionine | 1.19 | 0.00000603 |
| DAG(C18:1, C18:2) | 1.31 | 0.00000723 |
| Ceramide(d18:1, C24:0) | 1.27 | 0.0000123 |
| Proline | 1.25 | 0.0000167 |
| Tyrosine | 1.20 | 0.0000308 |
| Threonine | 1.19 | 0.00004 |
| Prostaglandin E2 | 2.10 | 0.0000604 |
| Hypoxanthine | 1.31 | 0.000323867 |
| 12-Hydroxyheptadecatrienoic | 2.20 | 0.000533193 |
| acid (C17:[5,8,10]3) | | |
| Tryptophan | 1.12 | 0.001259611 |
| Adrenaline (Epinephrine) | 0.60 | 0.001958413 |
| Erythronic acid | 1.16 | 0.002315092 |
| Serotonin (5-HT) | 0.70 | 0.002788449 |
| 14,15-Dihydroxyeicosatrienoic | 1.25 | 0.002888012 |
| acid (C20:cis[5,8,11]3) | | |
| Ceramide(d18:1, C24:1) | 1.18 | 0.003152822 |
| Histamine | 1.33 | 0.00619612 |
| Dopamine | 0.66 | 0.006480578 |
| Lactaldehyde | 0.63 | 0.006839905 |
| Sphingomyelin (d18:2, C18:0) | 1.07 | 0.008446022 |
| Glucose, lipid fraction | 0.86 | 0.017152437 |
| Lysophosphatidylcholine (C18:2) | 1.08 | 0.027186961 |
| Indole-3-lactic acid | 1.07 | 0.043110265 |
| Phosphatidylcholine(C18:1, C18:2) | 1.00 | 0.046906114 |
| Thromboxane B2 | 1.58 | 0.048511883 |
| Pantothenic acid | 1.13 | 0.049043122 |
| Cholesterylester hydroperoxide | 1.48 | 0.056126505 |
| (C18:2-9-OOH) | | |
| Cholesterylester hydroperoxide | 1.48 | 0.056126505 |
| (C18:2-13-OOH) | | |
| Cholesterylester hydroperoxide | 1.48 | 0.056126505 |
| (C20:4-OOH) | | |

TABLE 8a
Preferred biomarkers indicating quality issue in serum samples related
to pro-longed coagulation of blood: Selection based on assayability.
Biomarker (Metabolite)

Glycerol-3-phosphate, polar fraction
Arginine
Ornithine
Glutamate
Cysteine
Aspartate
Glycerate
Asparagine
Taurine
Cystine
Threonic acid
Maltose
Hypoxanthine

TABLE 8b
Preferred biomarkers indicating quality issue in serum samples related
to pro-longed coagulation of blood: Selection based on performance.
Biomarker (Metabolite)

Malate
Glycerol-3-phosphate, polar fraction
Pyruvate
Arginine
Glucose-1-phosphate
5-Oxoproline
Ornithine
Mannose
Glutamate
Cysteine
8-Hydroxyeicosatetraenoic acid (C20:trans[5]cis[9,11,14]4)
(8-HETE)
alpha-Ketoglutarate
Aspartate

TABLE 8c
Preferred biomarkers indicating quality issue in
serum samples related to pro-longed coagulation
of blood: Selection based on method “GC-polar”.
Biomarker (Metabolite)

Malate
Glycerol-3-phosphate, polar fraction
Pyruvate
Glucose-1-phosphate
5-Oxoproline
Ornithine
Mannose
Glutamate
Cysteine
alpha-Ketoglutarate
Aspartate
Serine
Phenylalanine
Phosphate (inorganic and from organic phosphates)
Glycerate
Glycine
Alanine
Asparagine
Lysine
Xanthine
myo-Inositol
Leucine
Histidine
Erythrol
Cystine
Mannosamine
Threonic acid
Glucosamine
Maltose
Valine
Ketoleucine
Isoleucine
Methionine
Proline
Tyrosine
Threonine
Hypoxanthine
Erythronic acid

TABLE 9
Preferred biomarkers indicating a specific quality issue in plasma or serum samples: Selection
based on criterion “uniqueness”: Biomarkers (Metabolites) with unique occurrence in
one of Tables 1 to 8 and the respective quality issue (confounder) they are indicative for.

| Biomarker (Metabolite) | Table | Quality Issue related to (Confounder) |
|---|---|---|
| Quinic acid | 1 | increased processing time of plasma samples |
| Cholesta-2,4,6-triene | 1 | increased processing time of plasma samples |
| TAG(C16:0, C18:1, C18:2) | 1 | increased processing time of plasma samples |
| Sorbitol | 1 | increased processing time of plasma samples |
| Arabinose | 1 | increased processing time of plasma samples |
| Lauric acid (C12:0) | 1 | increased processing time of plasma samples |
| Erucic acid (C22:cis[13]1) | 1 | increased processing time of plasma samples |
| Creatinine | 1 | increased processing time of plasma samples |
| Pentoses | 2 | increased processing time of blood samples |
| Fructose | 2 | increased processing time of blood samples |
| Metanephrine | 2 | increased processing time of blood samples |
| Dehydroepiandrosterone sulfate | 2 | increased processing time of blood samples |
| Glucuronic acid | 2 | increased processing time of blood samples |
| Glycochenodeoxycholic acid | 2 | increased processing time of blood samples |
| Citrate | 2 | increased processing time of blood samples |
| Ornithine to Arginine intra-sample ratio | 2 | increased processing time of blood samples |
| 5-O-Methylsphingosine (d16:1) | 3 | hemolysis |
| Sarcosine | 3 | hemolysis |
| Threitol | 3 | hemolysis |
| 4-Hydroxy-3-methoxymandelic acid | 3 | hemolysis |
| Docosapentaenoic acid | 3 | hemolysis |
| (C22:cis[4,7,10,13,16]5) | | |
| Taurochenodeoxycholic acid | 4 | microclotting |
| Indole-3-propionic acid | 4 | microclotting |
| 3-Indoxylsulfate | 4 | microclotting |
| scyllo-Inositol | 5 | contamination with white blood cells |
| Hydroxyhexadecenoylcarnitine | 5 | contamination with white blood cells |
| Oxalate | 5 | contamination with white blood cells |
| TAG (C16:0, C18:1, C18:3) | 5 | contamination with white blood cells |
| Phosphatidylcholine hydroperoxide | 6 | storage |
| (C16:0, C18:2-OOH) | | |
| Phosphatidylcholine hydroperoxide | 6 | storage |
| (C16:0, C18:1-OOH) | | |
| Phosphatidylcholine hydroperoxide | 6 | storage |
| (C18:0, C18:2-OOH) | | |
| Triacylgyceride hydroperoxide | 6 | storage |
| (C16:0, C18:1, C18:3-OOH) | | |
| Triacylgyceride hydroperoxide | 6 | storage |
| (C16:0, C18:2, C18:2-OOH) | | |
| Triacylgyceride hydroperoxide | 6 | storage |
| (C16:0, C18:1, C18:2-OOH) | | |
| Triacylgyceride hydroperoxide | 6 | storage |
| (C18:1, 18:2, C18:2-OOH) | | |
| Triacylgyceride hydroperoxide | 6 | storage |
| (C16:0, C18:1, C20:4-OOH) | | |
| Triacylgyceride hydroperoxide | 6 | storage |
| (C18:1, C18:1, C18:3-OOH) | | |
| 13,14-Dihydro-15-ketoprostaglandin D2 | 6 | storage |
| delta-12-Prostaglandin J2 | 6 | storage |
| 4-Hydroxyphenylpyruvate | 6 | storage |
| Lipoxin A4 | 6 | storage |
| 8,9-Epoxyeicosatrienoic acid | 6 | storage |
| (C20:cis[5,11,14]3) | | |
| Prostaglandin J2 | 6 | storage |
| Diacylglceride (C18:1, C18:2) | 6 | storage |
| 6-Oxoprostaglandin F1 alpha | 6 | storage |
| 5-Hydroxyeicosatetraenoic acid | 6 | storage |
| (C20:trans[6]cis[8,11,14]4) | | |
| 15-Deoxy-delta(12,14)-prostaglandin J2 | 6 | storage |
| Leukotriene B4 | 6 | storage |
| 17,18-Epoxyarachidonic acid | 6 | storage |
| (C20:cis[5,8,11,14]4) | | |
| 8-Hydroxyeicosatetraenoic acid | 6 | storage |
| (C20:trans[5]cis[9,11,14]4) | | |
| Corticosterone | 6 | storage |
| 14,15-Epoxyeicosatrienoic acid | 6 | storage |
| (C20:cis[5,8,11]3) | | |
| Triacylglyceride (C18:2, C18:3) | 6 | storage |
| Canthaxanthin | 6 | storage |
| Cryptoxanthin | 6 | storage |
| 11,12-Epoxyeicosatrienoic acid | 6 | storage |
| (C20:cis[5,8,14]3) | | |
| Salicylic acid | 7 | slow freezing of samples |
| Phosphatidylcholine (C18:0, C20:4) | 7 | slow freezing of samples |
| Xanthine | 8 | prolonged coagulation of blood |
| Glucosamine | 8 | prolonged coagulation of blood |
| 5-Hydroxy-3-indoleacetic acid (5-HIAA) | 8 | prolonged coagulation of blood |
| DAG (C18:1, C18:2) | 8 | prolonged coagulation of blood |

1.-26. (canceled)

27. A method for assessing the quality of a biological sample comprising the steps of:
(a) determining in said sample the amount of at least one biomarker from Tables 1, 1′, 2, 2′, 3, 3′, 4, 5, 5′, 6, 7, and/or 8; and
(b) comparing the said amount of the at least one biomarker with a reference, whereby the quality of the sample is assessed.

28. The method of claim 27, wherein the biological sample is assessed for prolonged processing of blood samples and wherein said at least one biomarker is from Table 1, 1′, 2 and/or 2′.

29. The method of claim 27, wherein the biological sample is assessed for hemolysis and wherein said at least one biomarker is from Table 3 or 3′.

30. The method of claim 27, wherein the biological sample is assessed for microclotting and wherein said at least one biomarker is from Table 4.

31. The method of claim 27, wherein the biological sample is assessed for contamination with blood cells and wherein said at least one biomarker is from Table 5 or 5′.

32. The method of claim 27, wherein the biological sample is assessed for improper storage and wherein said at least one biomarker is from Table 6.

33. The method of claim 27, wherein the biological sample is assessed for improper freezing and wherein said at least one biomarker is from Table 7.

34. The method of claim 27, wherein the biological sample is assessed for prolonged coagulation time of blood and wherein said at least one biomarker is from Table 8.

35. The method of claim 27, wherein said at least one biomarker is glutamate.

36. The method of claim 27, wherein said at least one biomarker is glycerate.

37. The method of claim 27, wherein step (a) is:
(a) determining in said sample the amount of at least one biomarker from Tables 1, 2, 3, 4, 5, 6, 7 and/or 8.

38. The method of claim 37, wherein the biological sample is assessed for prolonged processing of blood samples and wherein said at least one biomarker is from Table 1 and/or 2.

39. The method of claim 37, wherein the biological sample is assessed for hemolysis and wherein said at least one biomarker is from Table 3.

40. The method of claim 37, wherein the biological sample is assessed for microclotting and wherein said at least one biomarker is from Table 4.

41. The method of claim 37, wherein the biological sample is assessed for contamination with blood cells and wherein said at least one biomarker is from Table 5.

42. The method of claim 37, wherein the biological sample is assessed for improper storage and wherein said at least one biomarker is from Table 6.

43. The method of claim 37, wherein the biological sample is assessed for improper freezing and wherein said at least one biomarker is from Table 7.

44. The method of claim 37, wherein the biological sample is assessed for prolonged coagulation time of blood and wherein said at least one biomarker is from Table 8.

45. The method of claim 27, wherein said reference is derived from a sample or plurality of samples known to be of insufficient quality.

46. The method of claim 45, wherein an amount of the at least one biomarker in the sample being essentially identical to the said reference is indicative for insufficient quality, while an amount which differs therefrom is indicative for sufficient quality.

47. The method of claim 27, wherein said reference is derived from a sample or plurality of samples known to be of sufficient quality.

48. The method of claim 47, wherein an amount of the at least one biomarker in the sample being essentially identical to the said reference is indicative for sufficient quality, while an amount which differs therefrom is indicative for insufficient quality.

49. The method of claim 27, wherein said sample is a plasma, blood or serum sample.

50. A device for assessing the quality of a biological sample comprising:
(a) an analyzing unit for the said sample comprising a detector for at least one biomarker from Tables 1, 1′, 2, 2′, 3, 3′, 4, 5, 5′, 6, 7, and/or 8, preferably for at least one biomarker of Tables 1, 2, 3, 4, 5, 6, 7 and/or 8, said detector allowing for the determination of the amount of the said at least one biomarker in the sample; and operatively linked thereto,
(b) an evaluation unit comprising a data processing unit and a data base, said data base comprising a stored reference and said data processing unit having tangibly embedded an algorithm for carrying out a comparison of the amount of the at least one biomarker determined by the analyzing unit and the stored reference and for generating an output information based on which the assessment of the quality is established.

51. A kit for assessing the quality of a biological sample comprising a detection agent for at least one biomarker from Tables 1, 1′, 2, 2′, 3, 3′, 4, 5, 5′, 6, 7, and/or 8; preferably at least one biomarker from Tables 1, 2, 3, 4, 5, 6, 7 and/or 8 and, preferably, a reference for the said at least one biomarker.
